(19) United States
(12) Patent Application Publication
(10) Pub. No.: US 2025/0282818 A1
(43) Pub. Date: Sep. 11, 2025

(54) SYRBACTIN MACROLACTAMS AND UNNATURAL ANALOGS AS PROTEASOME INHIBITORS FOR THE TREATMENT OF MULTIPLE MYELOMA AND CHEMOENZYMATIC SYNTHESIS THEREOF

(71) Applicant: UNIVERSITY OF FLORIDA RESEARCH FOUNDATION,INCORPORATED, Gainesville, FL (US)

(72) Inventors: Hans Renata, Gainesville, FL (US); Alexander Amatuni, Gainesville, FL (US); Alexander Adibekian, Gainesville, FL (US); Anton Shuster, Gainesville, FL (US)

(73) Assignee: UNIVERSITY OF FLORIDA RESEARCH FOUNDATION,INCORPORATED, Gainesville, FL (US)

(21) Appl. No.: 18/859,979

(22) PCT Filed: Apr. 25, 2023
(86) PCT No.: PCT/US2023/019733

§ 371 Date: Oct. 24, 2024

Related U.S. Application Data
(60) Provisional application No. 63/334,625, filed on Apr. 25, 2022.

(51) Int. Cl. C07K 5/12; A61K 38/00

(52) U.S. Cl. CPC C07K 5/12; A61K 38/00

(57) ABSTRACT

One aspect of the invention is any compound, or salt thereof, described herein. Another aspect is a method of treating a disease, disorder, or symptom thereof, in a subject, comprising administration to the subject of a compound, or salt thereof, herein. Another aspect is a method of inhibiting a proteasome in a subject, comprising administration to the subject of a compound, or salt thereof, herein. Another aspect is a method of making a compound, or salt thereof, described herein using one or more reagents, chemical transformations, or chemical intermediate compounds as described herein.

### GOVERNMENT SUPPORT

This invention was made with government support under R35 GM128895 awarded by the National Institutes of Health. The government has certain rights in the invention.

### BACKGROUND

Background of the present disclosure can be found in Amatuni, A. and Renata, H., Org. Biomol. Chem. 2019 Feb. 13; 17(7): 1736-1739 and Amatuni, A. et al., Cell Chem. Biol. 2020 Oct. 15; 27(10): 1318-1326.e18, which are incorporated by reference in their entirety.

### BRIEF DESCRIPTION OF THE DRAWINGS

The present invention is further described below with reference to the following non-limiting examples and with reference to the following figures, in which:

FIG. 1 depicts representative structures of syrbactin natural products and clinical proteasome inhibitors.

FIG. 2 depicts a chemoenzymatic route to cepafungin I.

FIG. 3 depicts a preliminary series of 1 analogs and comparison to prior syringolin analogs.

FIG. 4 depicts the bound crystal structure of cepafungin I at β5 subunit of yeast 20S proteasome. PDB ID: 4FZC.⁸ Dashed lines indicate polar contacts. The catalytic β5N-terminal threonine covalently bonded to the cepafungin macrocycle is labeled “T1”. β5 residues involved in polar contacts are Gly47, Ala49, Thr21 and Asp126. A hydrophobic channel for the tail fragment involves β6 subunit residues Pro127, Val128, Pro104, Tyr106 and Tyr5.

FIG. 5 depicts synthesis of cepafungin analogs 40-51.

FIGS. 6A-6E depict biological evaluation of cepafungin and analogs in RPMI 8226 cells. FIG. 6A depicts proteasome inhibition screening at 30 nM compound concentration with subunit-specific fluorogenic proteasome substrates Suc-LLVY-AMC (β5) and Ac-RLR-AMC (β2). FIG. 6B depicts cytotoxicity screening at 30 nM and 100 nM compound concentration. FIG. 6C depicts EC₅₀ measurement of 50 in comparison to 1. FIG. 6D depicts proteasome subunit IC₅₀ measurements of 50 in comparison to 1. FIG. 6E depicts a comparison of phenyl cepafungin 50 and bortezomib in human MM (RPMI8226, U266, H929) and MCL (JeKo-1, HBL2, Mino) cell lines.

FIG. 7 depicts cytotoxicity EC₅₀ measurements for 50 and bortezomib (BTZ) in MM and MCL cell lines.

FIG. 8 depicts cytotoxicity EC₅₀ measurements for phenyl cepafungin (50) and hybrid analog (S39) in RPMI 8226.

FIG. 9 depicts the bound crystal structure of cepafungin I at β2 subunit of yeast 20S proteasome. PDB ID: 4FZC.^{S3} Dashed lines indicate polar contacts. The catalytic β2N-terminal threonine covalently bonded to the cepafungin macrocycle is labeled “T1”. 02 residues involved in polar contacts are Gly47, Ala49, Thr21 and Asp124. A hydrophobic channel for the tail fragment involves β3 subunit residues Leu125, Ile126, Pro101 and Phe103.

### DETAILED DESCRIPTION

### 5.1—Introduction

Considering that cepafungin I (1) is the most potent proteasome inhibitor among the syrbactins, has low nanomolar inhibitory activity towards both β2 and β5 subunits, and engages the 20S proteasome with exceptional selectivity in a MM cell line, this natural product was selected as a promising scaffold for further SAR optimization towards a new anticancer agent.

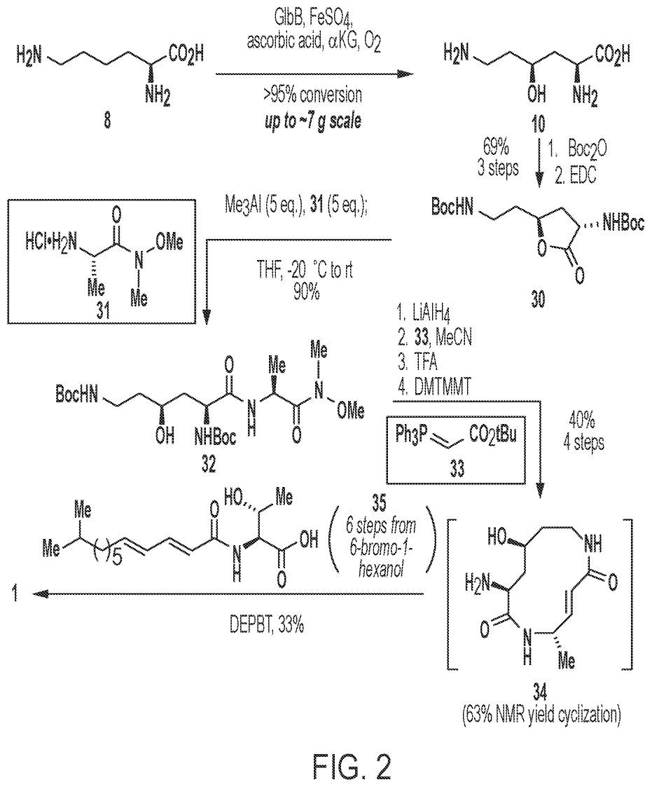

Syntheses of syringolins A (4) and B have been reported by Kaiser,¹ Stephenson,² Pirrung³ and Ichikawa,⁴ and Schmidt in 1992 achieved the first total synthesis of glidobactin A (2).⁵ Early structure-activity relationships (SAR) were established with semi-synthetic glidobactin analogs harboring unnatural tail fragments, though the syrbactins' mechanism of action had not been elucidated at the time.⁶ More recent studies on the syringolins and unnatural analogs have elucidated their activity in the context of proteasome inhibition. Collectively, these studies have revealed some key factors contributing to the syrbactins' potent bioactivity. The presence of longer lipophilic tails seen in glidobactins, cepafungins and synthetic analogs versus those of natural syringolins has been shown to result in greater proteasome inhibition in vitro, likely by increased hydrophobic interactions distal to the active site.^{4,7} Notably, a single methyl branching at the end of the cepafungin I (1) fatty acid leads to ca. 5-fold improvement of β5-subunit inhibition compared to glidobactin A (2).⁸ Further exploration of hydrophobic functionalities at this position may further improve the proteasome binding affinity at one or more subunits. Larger hydrophobic residues in place of the macrocycle-adjacent threonine are tolerated by the S3 subsite and can improve binding affinity.⁴ However, other β-hydroxy amino acids in place of threonine have not been explored, nor has there been a direct comparison between threonine and its non-hydroxylated counterpart. Interestingly, several bacteria produce glidobactin-like syrbactins, and several other bacteria have bioinformatically been shown to harbor syrbactin-like gene clusters.^{9,10} While several lysine hydroxylases are found in nature, syrbactins with different lysine hydroxylation patterns have not been found, except for minor amounts of desoxy-glidobactins known as “luminmycins” from Photorhabdus laumondii, such as luminmycin A (3).¹¹ Given the cepafungins' potent bioactivity and the current gap in SAR data, we sought to utilize our chemoenzymatic strategy (FIG. 2) to access analogs harboring modifications at all regions of the scaffold. Unique in our approach is the utilization of several enzymes for site-selective C—H hydroxylation of amino acids, facilitating rapid exploration of chemical space around the cepafungin macrocycle and tail fragment linker. This approach has resulted in the first comprehensive SAR analysis of cepafungin's proteasome inhibitory activity and five unnatural analogs with improved bioactivity.

### 5.2—Considerations for Analog Design

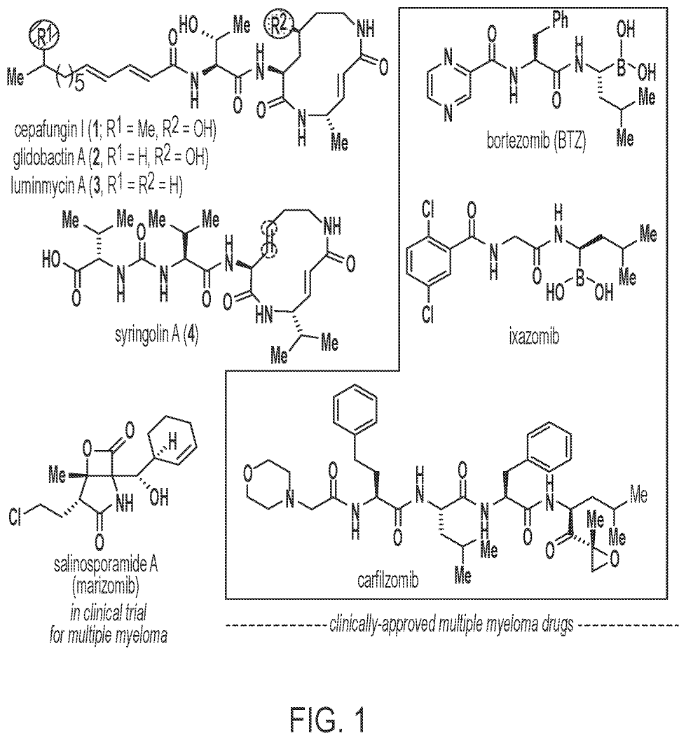

Early syntheses of the syringolins ultimately led to the development of various lipophilic analogs with improved cytotoxicity and proteasome inhibition. Ichikawa's analog 36 and Pirrung's analog 37 both benefited from replacement of one or more C-terminal Val residues in the syringolin A tail (FIG. 1).⁴² Ichikawa reported that a Phe macrocycle linker was found to increase β5 inhibition 15-fold compared to syringolin A, likely by improved binding at the hydrophobic pocket of the S3 subsite, although this analog still exhibited low cytotoxicity. Exploration of cell-permeable lipid tails led to the discovery of analog 36 which exhibited low nanomolar cytotoxicity. Likewise, Pirrung reported a syringolin A analog 37 containing a dodecanoyl-ureido-Val tail fragment. This analog had inhibitory activity comparable to ixazomib, displays lower resistance index in MM cell lines than bortezomib, and prolonged survival in MM mouse xenografts.¹² However, prior SAR studies on syrbactins have not explored analogs that are more cepafungin/glidobactin-like.

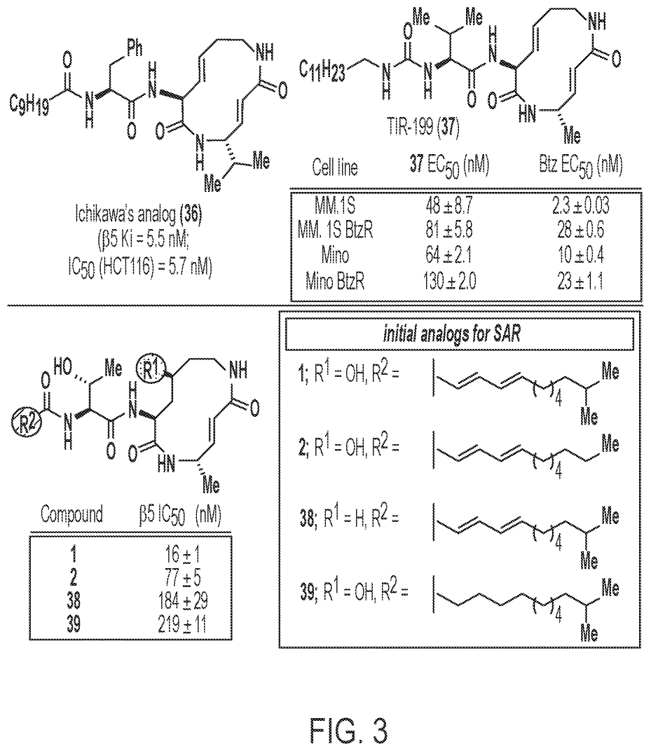

Our preliminary series of analogs (FIG. 3), including the natural product glidobactin A (2), were synthesized by largely the same route (FIG. 2).¹³ Desoxycepafungin (38) was synthesized from Boc-Lys(Boc)-OH to assess the role of the macrocycle hydroxylation in 1 towards proteasome inhibition. The saturated cepafungin analog 39 was designed to address the possible role of rigidity in the tail fragment induced by the diene seen in many natural cepafungins and glidobactins. An analog bearing unsaturation at its macrocyclic lysine was envisioned to provide a head-to-head comparison between the cepafungin and syringolin A (4) macrocyclic core. However, dehydration conditions on the hydroxylated macrocycle resulted in decomposition and inseparable mixtures of olefin isomers. Proteasome subunit β5 IC₅₀ values were determined in situ in RPMI 8226 cells (FIG. 3). In close agreement with prior measurement of β5 IC₅₀ for natural 1 and 2 in purified yeast proteasome,⁸ in our hands the additional branching methyl in 1 indeed leads to ca. 5-fold improved IC₅₀ compared to 2 in RPMI 8226 cells. Notably, the macrocyclic hydroxyl group in 1 leads to ca. 11-fold improved β5 inhibition compared to its “desoxy-cepafungin” analog 38. Likewise, saturation of the fatty acid fragment in 39 causes ca. 14-fold decrease in β5 inhibition. This preliminary series of analogs highlighted that the macrocyclic hydroxylation, fatty acid unsaturation, and terminal lipid functionality are critical for potent proteasome inhibitory activity.

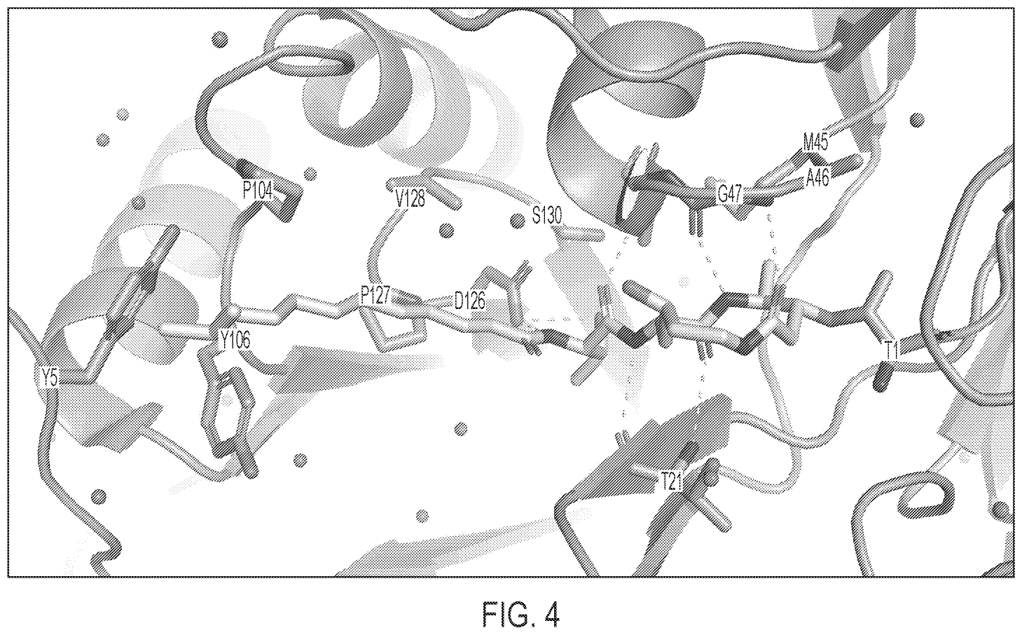

To gain a more comprehensive understanding of cepafungin's SAR, we designed a broader series of analogs guided by a previous crystal structure of yeast proteasome:cepafungin complex⁸ (FIG. 4), insights from prior SAR studies on related syrbactins,^{4,6,7,14,15} and our preliminary SAR data. Based on these insights, four divergence points were identified, namely the oxygenation pattern of the macrocycle, the vinylogous amino acid residue in the macrocycle, the side-chain of the β-OH amino acid linker, and the terminal functionality of the lipid tail. Regarding the first point, we envisioned L-lysine (8) as the key divergence point in analog generation (FIG. 2). That desoxycepafungin (38) has ca. 11-fold higher IC₅₀ than 1 just from omission of the lysine hydroxylation raises the question whether the reduced potency arises from a general decrease in polarity of the macrocycle or from a more specific electrostatic interaction in the active site. Notably, no hydrogen bonds or water molecules are seen near the hydroxy group of 1 and the β5 subunit of yeast proteasome in the crystal structure (FIG. 4). To probe the relevance of this hydroxylation, we sought to compare analogs containing different lysine oxidation patterns. The (2S,4R) diastereomer of 10 could be prepared via KDO3, an Fe/αKG enzyme from Flavobacterium johnsoniae that performs regio- and stereoselective (R)—C4-hydroxylation on free lysine. Elaboration to the macrocycle would then follow the general route outlined in FIG. 2. Additionally, the (2S,3S)-3-hydroxylysine regioisomer could be prepared with KDO1, an Fe/αKG enzyme from Catenulispora acidiphila that performs selective (S)—C3-hydroxylation on free lysine.¹⁶ As this monomer is not expected to undergo spontaneous lactonization upon amine protection, the protected 3-hydroxy amino acid could be directly coupled to an Ala derivative for elaboration to the macrocycle.

The second divergence point is the vinylogous amino acid residue in the macrocycle (FIG. 2). The naturally occurring syringolins incorporate an L-Val residue in place of the L-Ala seen in the glidobactins and cepafungins, and in both cases these sidechains occupy the S1 subsite at the β5 subunit. However, a direct comparison between cepafungin and the Val-substituted macrocycle analog has yet to be reported. The third divergence point is the β—OH amino acid adjacent to the macrocycle, which for cepafungins and glidobactins is canonically occupied by an L-Thr residue. Late-stage introduction of this residue in the tail fragment synthesis facilitates the incorporation of other β-OH amino acids. Notably, the hydroxy group of Thr is involved in hydrogen bonding with Asp126 at the S3 subsite, which comprises a prominent pocket adjacent to the methyl group of Thr and can accommodate larger functional groups (FIG. 4). Ichikawa's syringolin analogs incorporated fatty-acyl-Phe residues in place of the canonical bis-ureido-Val (FIG. 3) and resulted in greater proteasome inhibition.⁴ However, direct comparison of cepafungin I against analogs containing desoxy-Thr or other β-OH amino acids has not been performed. Such β—OH amino acids can be prepared by enzymatic or chemical methods.

Finally, our preliminary SAR results (FIG. 3) indicate that the diene functionality in the fatty acid as well as the terminal methyl branching are very important for bioactivity. The diene may assist in orienting the lipid tail along a hydrophobic patch on the adjacent β6 subunit and between residues Pro127 and Val128 (FIG. 4). Additionally, early cytotoxicity assays and more recent studies on glidobactin-like natural products highlight that C₁₂ fatty acid chains in the tail fragment are the optimal length for potent bioactivity.^{6,11} Therefore, additional analogs were designed to keep the C₁₂ chain length and the diene moiety but explore alternate groups in place of the terminal isopropyl in 1. Substitutions can readily be made by variations of the Grignard reagent during the initial Kochi coupling to 6-bromo-1-hexanol. Notably, the cepafungin lipid terminates near β6 subunit residues Tyr5 and Tyr106, which appear poised to facilitate potential pi-stacking interactions (FIG. 4). Thus, we sought an analog with a phenyl ring at the lipid terminus to induce pi-stacking with either of these residues. Larger hydrophobic groups also include tert-butyl, cyclopentyl and cyclohexyl. Additionally, as trifluoromethyl groups may prefer interactions with Phe, Met, Leu and Tyr residues compared to the corresponding methyl-substituted compound,¹⁷ an analog containing a CF₃-substituted lipid fragment was sought. Direct adaptation of our original synthetic route to this analog would require the introduction of a terminal hexafluoroisopropyl moiety by Wittig olefination.¹⁸ Due to operational hazards involved in working with highly volatile and toxic hexafluoroacetone, we opted to instead make the CF₃-substituted lipid fragment via photocatalytic trifluoromethylation of 5-hexen-1-ol,¹⁹ followed by further elaboration based on our synthetic route to cepafungin. Taken together, this expanded series of analogs spans modifications of the macrocycle, tail fragment β—OH amino acid, and fatty acid fragments. Importantly, the modularity of our synthesis would readily allow combinations of fragment building blocks after initial screening of mono-substitutions to identify any synergistic effects.

### 5.3—Syntheses of 13 Novel Cepafungin Analogs

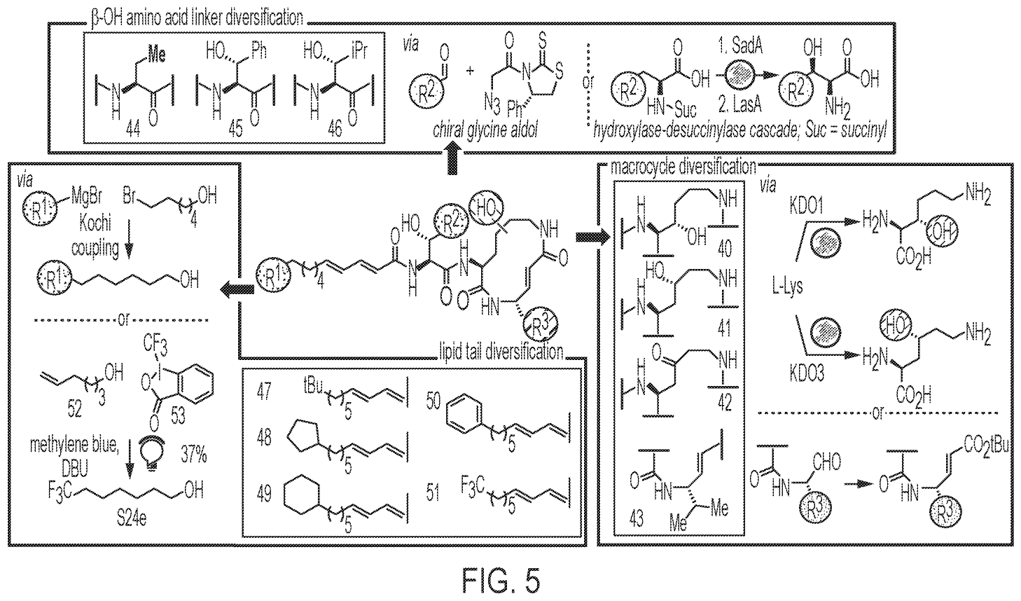

In our initial attempts to hydroxylate lysine with KDO3, full consumption of starting material was observed with 2 equivalents of αKG co-substrate, but no desired hydroxylysine product could be isolated by ion-exchange resin or by preparative TLC after Fmoc derivatization. NMR and LCMS analysis indicated primarily decomposition of the starting material. With 1.25 equivalents of αKG co-substrate, LCMS analysis indicated cleaner full conversion to the desired hydroxylated product. Upon scale-up, performing the reaction with 20 mM Lys at pre-lysis OD₆₀₀=10 revealed the overoxidized keto lysine S13 as the major product (32%), and (2S,4R)-4-hydroxylysine S11 (11%) and lactone S12 (6%) as minor products. To our knowledge, this reactivity pattern for a lysine hydroxylase was not described before in the initial disclosure of KDO3. To favor mono-oxidation, the reaction was performed with 40 mM lysine substrate, 1.25 equivalents of αKG and pre-lysis OD₆₀₀=4.3 (see Table 5.S1 for optimization). On gram scale, these conditions provided a combined 42% 2-step yield of (2S,4R)-4-hydroxylysine S11 and its spontaneously lactonized counterpart S12, and 12% of keto-lysine byproduct S13 after Boc protection (Scheme 5.S1). Lactone S12 was carried forward as in FIG. 2 to access the epi-4-OH cepafungin analog 40 (FIG. 5).

While the keto-lysine product S13 was unexpected, it raised a question of whether a cepafungin macrocycle harboring a C4-ketone in place of a hydroxyl would be beneficial. We envisioned that the ketone sp² center could introduce additional strain to the 12-membered ring, potentially making the α,β-unsaturated amide more reactive. Additionally, this substitution would swap a hydrogen bond donor for an acceptor. The protected keto-lysine byproduct could be coupled directly to an Ala derivative towards the keto-macrocycle analog, though we opted instead to directly oxidize intermediate S17c from the natural product synthesis. Subsequent deprotection and macrocyclization proceeded in similar yields as in cepafungin I, despite the additional sp² center. KDO1 was used for selective C3 oxidation of lysine as described in the literature.²⁰ After peptide coupling to 31, subsequent steps followed those of the natural product synthesis to provide 41 in good yield. The valinyl cepafungin analog 43 was prepared by aminolysis of lactone 30 with a dimethylaluminum amide reagent generated from Val derivative S14b. Gratifyingly, the aminolysis conditions developed in the total synthesis tolerated the bulkier Val sidechain and gave excellent yield of dipeptide S15c on gram scale.

For diversification of the β-OH amino acid linker, the desoxy-Thr variant 44 was synthesized from commercial (S)-ethylglycine. Towards β—OH-Phe 45, Franck's auxiliary-based aldol chemistry²¹ readily allowed the union of a chiral glycine equivalent to benzaldehyde. The addition product was directly subjected to methanolysis in one pot to cleave the auxiliary and form methyl ester S34 in 66% yield over 2 steps. Catalytical hydrogenation and subsequent couplings to the unsaturated fatty acid and core macrocycle completed the synthesis of β-OH-Phe cepafungin 45. Next, we targeted β-OH-Leu to compare the natural product against both larger aromatic and aliphatic β-OH amino acids at the S3 subsite. Unfortunately, the Franck aldol reaction did not produce any of the desired adduct with either isobutyraldehyde or methacrolein. Instead, we turned to the Fe/αKG dioxygenase SadA, which performs stereoselective β-hydroxylation on a variety of aliphatic N-succinyl amino acids, as well as LasA, an N-desuccinylase enzyme from the same producing organism for a cascade synthesis of β-hydroxy-Leu.²² While the original report for the enzymes describes a one-pot transformation with both enzymes, we opted for flash C18 purification of intermediate S36 to facilitate optimization of the LasA reaction that had initially failed to produce the desired product or with only minimal conversion. Ultimately, 10 mM loading of N-succinyl-β-hydroxy-Leu substrate and 20° C. reaction with LasA at pre-lysis OD₆₀₀=20 promoted nearly complete conversion to free β-hydroxy-Leu, isolated in 81% yield as the Fmoc derivative over two steps. Fatty acid analogs 47-50 were prepared by Kochi couplings of 6-bromo-1-hexanol with the corresponding Grignard reagents, then carried forward following the cepafungin I route. Finally, the CF₃ fatty acid derivative 51 was prepared by way of alcohol S24e, made via photocatalytic trifluoromethylation of 52 with Togni reagent II (53).¹⁹ In total, 12 analogs were synthesized in this campaign.

### 5.4—In Vitro Biological Evaluation

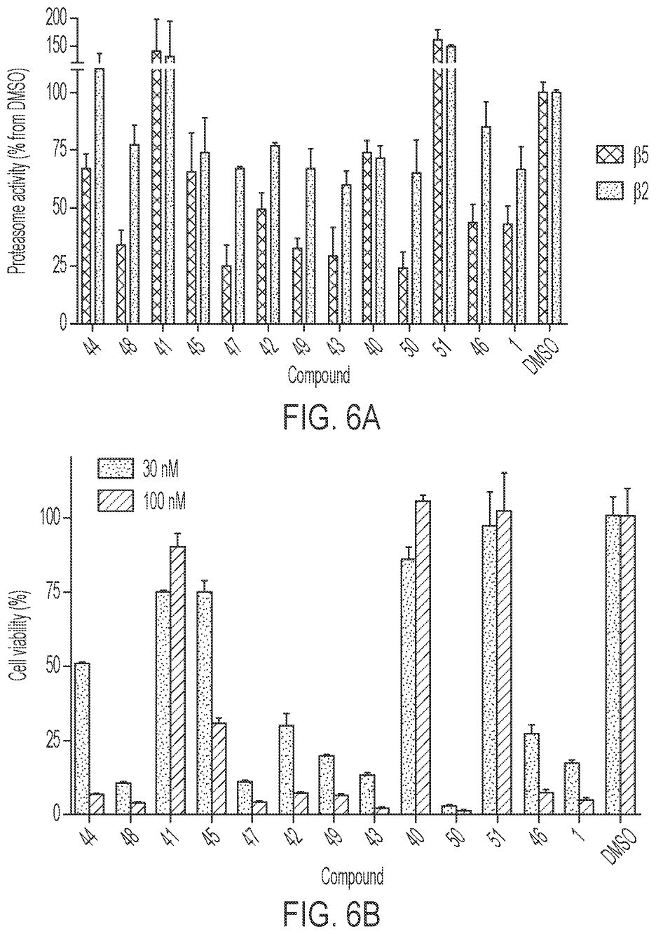

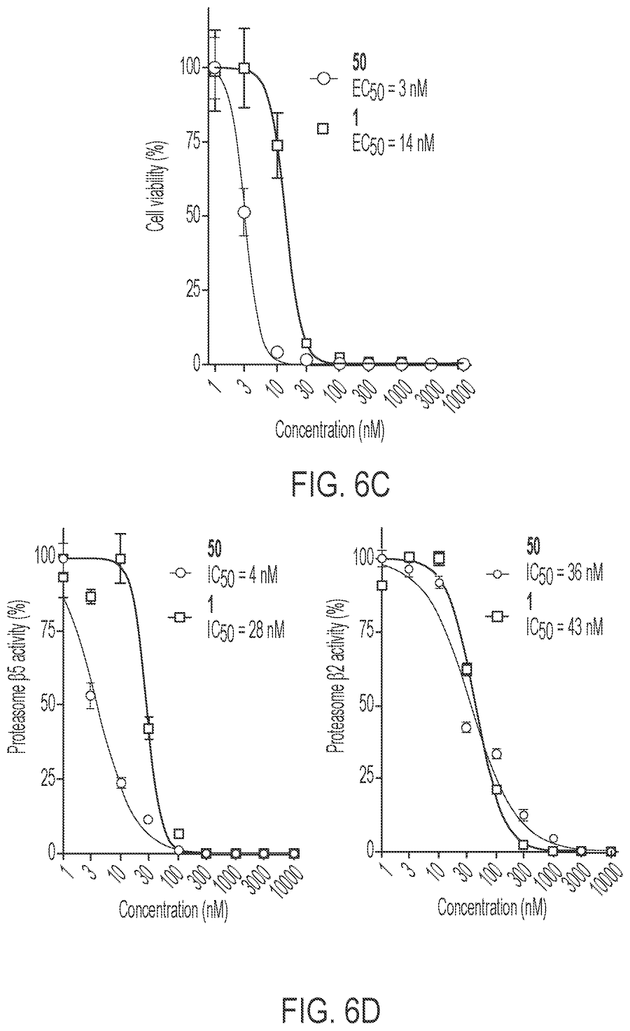

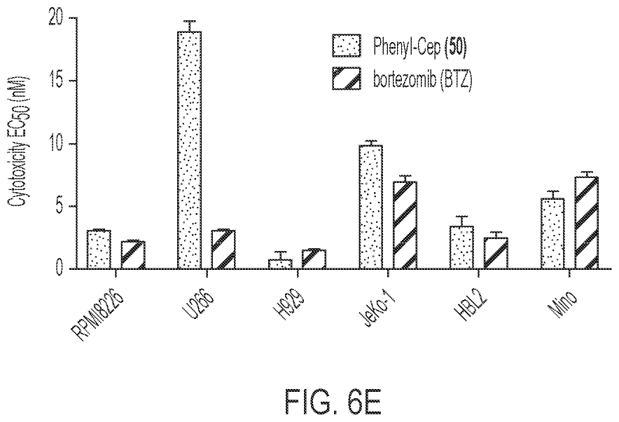

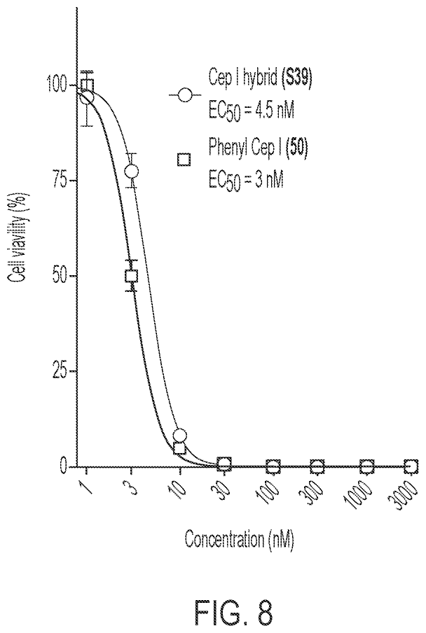

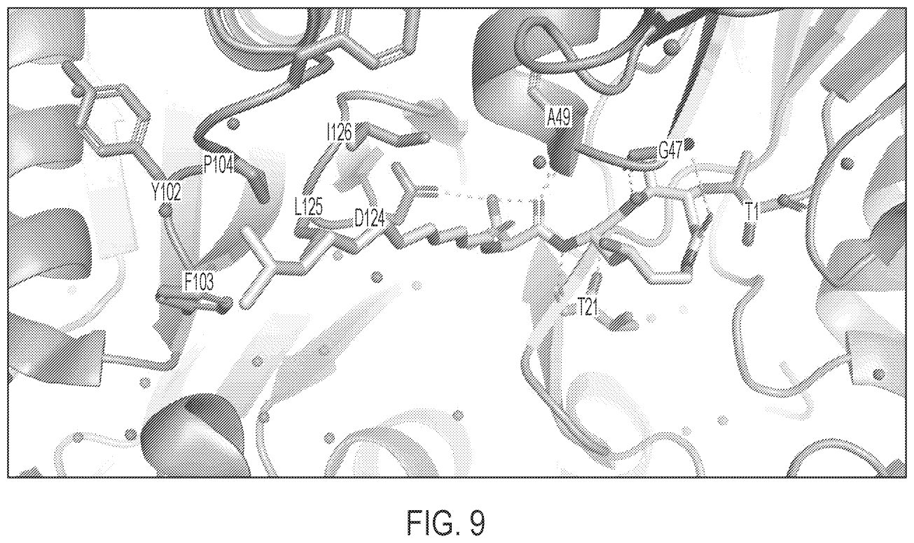

Cellular assays were performed with human multiple myeloma cell line RPMI 8226. First, relative inhibition of proteasome subunits β5 and β2 for all analogs and the natural product was measured at 30 nM compound concentration. Following incubation of the analogs in cell culture for 6 hours, cells were lysed and fluorogenic subunit-specific peptide substrates Suc-LLVY-AMC (β5) or Ac-RLR-AMC (β2) were incubated with the lysates, and fluorescence readings provided relative proteasome activity levels with respect to DMSO (FIG. 6A). Next, cytotoxicity screening was performed at 30 nM and 100 nM concentrations for better coverage of dynamic range (FIG. 6B). From these results, cytotoxicities track closely with relative proteasome inhibition, suggesting that proteasome inhibition is a primary cause of cytotoxicity. Notably, compounds 43, 47, 48, and 50 are more potent than the parent natural product 1, with phenyl cepafungin 50 being the most potent. EC₅₀ measurement in RPMI 8226 revealed that 50 has ca. 4-5 fold greater cytotoxicity than the parent cepafungin I 1 with an EC₅₀ of 3 nM (FIG. 6C). Proteasome subunit activity measurements revealed that 50 has 7-fold lower IC₅₀ than 1 for the β5 subunit and roughly similar IC₅₀ for the β2 subunit (FIG. 6D). Therefore, the improved cytotoxicity of phenyl cepafungin 50 likely results from a more pronounced loss of β5 function in RPMI 8226 cells. This drastic increase in proteasome inhibition may be due to pi-stacking of the lipid tail phenyl ring with the β6 subunit residues Tyr106 and/or Tyr5. The β2 subunit-bound natural product has residues Tyr102 and Phe103 on the adjacent β3 subunit oriented further away from the terminal lipid methyl groups, and presumably would not facilitate pi-stacking to the extent possible at the adjacent β6 subunit (see FIG. 4, FIG. 9). The tert-butyl and cyclopentyl cepafungins 47 and 48, respectively, also have improved cytotoxicity relative to 1 albeit to a lesser extent than 50, suggesting the aromatic moiety in the lipid tail is favored over large aliphatic groups. Cyclohexyl cepafungin 49 appears to have roughly similar activity as the natural product. Interestingly, valinyl cepafungin 43 displayed slightly greater cytotoxicity than the natural product and prompted the synthesis of a hybrid analog (S39) containing the phenyl cepafungin tail fragment and valinyl cepafungin macrocycle. These combined modifications were made in hopes that the effects of both substitutions would be additive. While this hybrid analog was significantly more potent than the natural product with an EC₅₀ of 4.5 nM against RPMI 8226, its cytotoxicity did not surpass that of 50 (FIG. 8). Of the other modified macrocycle analogs, only keto cepafungin 42 displayed potency approaching that of the natural product, while epi-4-OH (40) and 3-OH cepafungin (41) were nearly inactive at both concentrations. This result indicates that the regio- and stereochemistry of lysine hydroxylation in the cepafungin and glidobactin biosynthses is critical for potent proteasome inhibition. The presence of a Thr residue next to the cepafungin macrocycle appears to be favored over the ethyl sidechain of desoxy-Thr analog 44 as indicated by cytotoxicity and hydrogen-bonding seen in the cepafungin bound crystal structure (FIG. 3, 4B). However, the β-OH-Phe analog 45 was substantially weaker, while the β-OH-Leu analog 46 approached the potency of the natural product. The trifluoromethyl analog 51 was essentially inactive at both concentrations tested.

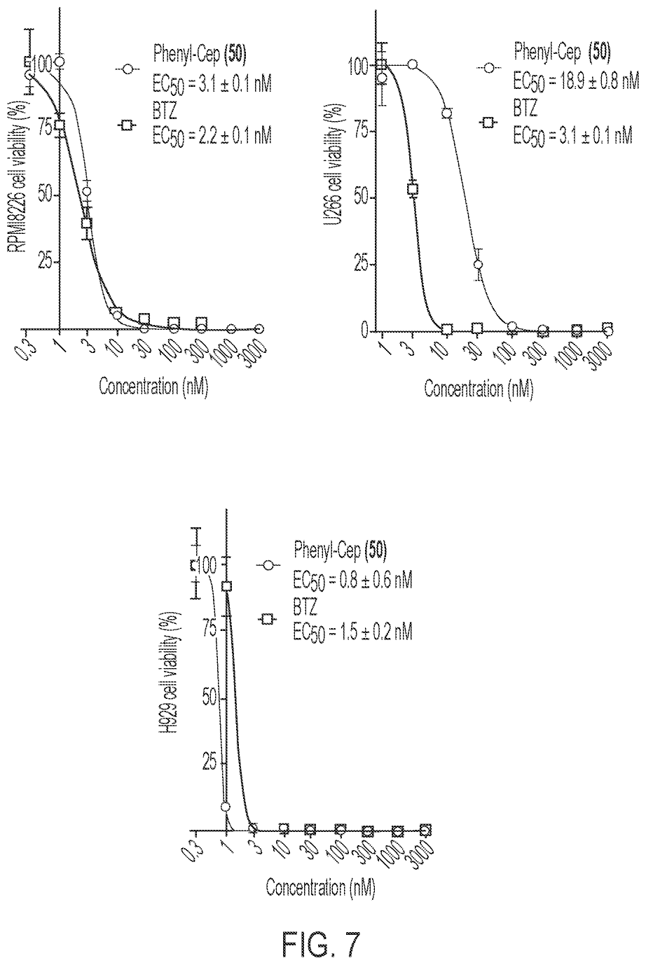

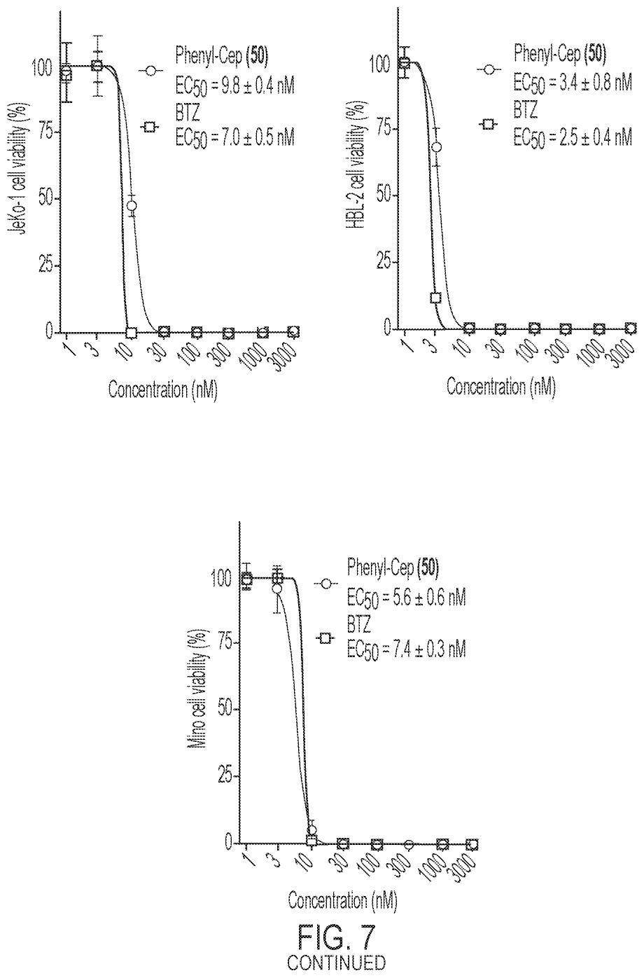

Finally, we sought to compare the new lead analog (50) against the clinical drug bortezomib. It has been shown that in addition to MM, mantle cell lymphoma (MCL) is also sensitive to proteasome inhibition. Indeed, bortezomib itself is FDA-approved for the treatment of MCL.²³ Therefore, we compared 50 and bortezomib in 3 different cell lines of both MM and MCL (FIG. 6E, FIG. 7). Gratifyingly, phenyl cepafungin displayed similarly low-nanomolar cytotoxicity in all cell lines as bortezomib except for U266, for which it has an EC₅₀ of ˜20 nM.

### 5.5—Conclusion

The chemoenzymatic strategies described herein enabled the first synthesis of cepafungin I. Early route exploration examined the use of the fungal Fe/αKG FoPip4H for site-selective C—H hydroxylation of L-pipecolic acid, which was followed by functional group interconversions to generate a 4-hydroxylysine surrogate. Unfortunately, all attempts at macrocyclization failed to provide the desired 12-membered macrolactam and instead resulted in the formation of a 6-membered cyclization product in low yield. This result underscored the challenge of forming this 12-membered ring and prompted a more biomimetic protecting group-free macrolactamization strategy, which led to our study on the glidobactin biosynthetic gene cluster to discover and characterize the native lysine hydroxylase. The Fe/αKG from the cluster, GlbB, hydroxylates L-lysine with exquisite regio-/stereoselectivity and very high total turnover, allowing for easy scale-up whereby ˜7 g of free L-lysine could be hydroxylated with 1 L of clarified lysate. By leveraging the high efficiency of this biocatalytic reaction, we completed the first synthesis of cepafungin I in 7.9% yield over 9 longest linear steps.

Prior to our work, a major knowledge gap in the bioactivity of cepafungin was its engagement of the proteasome in human MM cells and selectivity in targeting the proteasome over a multitude of other proteases. To address this, an alkyne-tagged probe analog was synthesized by our chemoenzymatic route and used for classical chemoproteomics studies. A combination of in-gel competitive profiling and in situ competitive LC-MS/MS based chemoproteomics demonstrated cepafungin's exceptional selectivity towards several proteasome subunits. Moreover, global proteomics experiments with 1 and bortezomib indicated a high degree of overlap in upregulated protein expression, suggesting a similar mechanism of action between the two molecules.

The modularity of our chemoenzymatic route enables facile modification of all regions of the natural product scaffold to gain comprehensive SAR data and its efficiency allows for routine scale up to provide ≥40 mg of final compounds. By taking advantage of these features, modifications to the natural product were made at three distinct regions of the scaffold, guided by proteasome-bound crystal structure and preliminary SAR data. The regio- and stereochemistry of lysine hydroxylation is critical for potent proteasome inhibition, whereby the configuration found in the parent natural product was found to be optimal for proteasome inhibition, despite a lack of obvious binding interactions in the crystal structure of yeast 20S proteasome:cepafungin complex. The S3 subsite appears to prefer aliphatic 3-OH amino acids linked to the macrocycle, and a larger aliphatic residue proximal to the macrocycle's reactive electrophile is tolerated. The diene moiety in the tail fragment is also essential for potent inhibition, as is the terminal functionality of the fatty acid. This work constitutes the most comprehensive SAR study on the cepafungin series to date, undoubtedly made possible by the high modularity of our chemoenzymatic route. One fatty acid analog, phenyl cepafungin (50), has 7-fold greater β5 inhibitory activity and exhibits similar cytotoxicity to the clinically approved drug bortezomib in several MM and MCL cell lines. Potent β5 and β2 co-inhibitory activity, as seen in 50 and other analogs, may serve as a viable means of overcoming bortezomib and carfilzomib resistance in multiple myeloma. In vivo mouse studies, evaluation against PI-resistant cancers, and further structural refinements on 50 are ongoing in our laboratories.

### 5.6—REFERENCES

1. (a) Clerc, J.; Groll, M.; Illich, D. J.; Bachmann, A. S.; Huber, R.; Schellenberg, B.; Dudler, R.; Kaiser, M. Proc. Natl. Acad. Sci. U.S.A 2009, 106, 6507-6512. (b) Clerc, J.; Schellenberg, B.; Groll, M.; Bachmann, A. S.; Huber, R.; Dudler, R.; Kaiser, M. European J. Org. Chem. 2010, No. 21, 3991-4003.
2. Dai, C.; Stephenson, C. R. J. Org. Lett. 2010, 12, 3453-3455.
3. Pirrung, M. C.; Biswas, G.; Ibarra-Rivera, T. R. Org. Lett. 2010, 12, 2402-2405.
4. Chiba, T.; Hosono, H.; Nakagawa, K.; Asaka, M.; Takeda, H.; Matsuda, A.; Ichikawa, S. Angew. Chemie—Int. Ed. 2014, 53, 4836-4839.
5. Schmidt, U.; Kleefeldt, A.; Mangold, R. J. Chem. Soc. Chem. Commun. 1992, No. 22, 1687-1689.
6. Oka, M.; Numata, K. ichi; Nishiyama, Y.; Kamei, H.; Konishi, M.; Oki, T.; Kawaguchi, H. J. Antibiot. (Tokyo). 1988, 41, 1812-1822.
7. Clerc, J.; Li, N.; Krahn, D.; Groll, M.; Bachmann, A. S.; Florea, B. I.; Overkleeft, H. S.; Kaiser, M. Chem. Commun. 2011, 47, 385-387.
8. Stein, M. L.; Beck, P.; Kaiser, M.; Dudler, R.; Becker, C. F. W.; Groll, M. Proc. Natl. Acad. Sci. U.S.A 2012, 109, 18367-18371.
9. Schellenberg, B.; Bigler, L.; Dudler, R. Environ. Microbiol. 2007, 9, 1640-1650.
10. Bian, X.; Huang, F.; Wang, H.; Klefisch, T.; Müller, R.; Zhang, Y. Chembiochem 2014, 15, 2221-2224.
11. Zhao, L.; Le Chapelain, C.; Brachmann, A. O.; Kaiser, M.; Groll, M.; Bode, H. B. ChemBioChem 2021, 22, 1582-1588.
12. Pierce, M. R.; Robinson, R. M.; Ibarra-Rivera, T. R.; Pirrung, M. C.; Dolloff, N. G.; Bachmann, A. S. Leuk. Res. 2020, 88 (July 2019).
13. Amatuni, A.; Shuster, A.; Adibekian, A.; Renata, H. Cell Chem. Biol. 2020, 27, 1318-1326.e18.
14. Kitahata, S.; Yakushiji, F.; Ichikawa, S. Chem. Sci. 2017, 8, 6959-6963.
15. Kitahata, S.; Chiba, T.; Yoshida, T.; Ri, M.; Iida, S.; Matsuda, A.; Ichikawa, S. Org. Lett. 2016, 18, 2312-2315.
16. Baud, D.; Saaidi, P. L.; Monfleur, A.; Harari, M.; Cuccaro, J.; Fossey, A.; Besnard, M.; Debard, A.; Mariage, A.; Pellouin, V.; et al. ChemCatChem 2014, 6, 3012-3017.
17. Abula, A.; Xu, Z.; Zhu, Z.; Peng, C.; Chen, Z.; Zhu, W.; Aisa, H. A. J. Chem. Inf Model. 2020, 60, 6242-6250.
18. Jewell, C. F.; Brinkman, J.; Petter, R. C.; Wareing, J. R. Tetrahedron 1994, 50, 3849-3856.
19. Pitre, S. P.; McTiernan, C. D.; Ismaili, H.; Scaiano, J. C. ACS Catal. 2014, 4, 2530-2535.
20. Zhang, X.; King-Smith, E.; Renata, H. Angew. Chemie—Int. Ed. 2018, 57, 5037-5041.
21. Patel, J.; Clave, G.; Renard, P. Y.; Franck, X. Angew. Chemie 2008, 120, 4292-4295.
22. Hibi, M.; Kasahara, T.; Kawashima, T.; Yajima, H.; Kozono, S.; Smirnov, S. V.; Kodera, T.; Sugiyama, M.; Shimizu, S.; Yokozeki, K.; et al. Adv. Synth. Catal. 2015, 357, 767-774.
23. Moore, B. S.; Eustiquio, A. S.; McGlinchey, R. P. Curr. Opin. Chem. Biol. 2008, 12, 434-440.

### 5.7—Experimental

### General Materials and Methods

Unless otherwise noted, all chemicals and reagents for chemical reactions were purchased at the highest commercial quality and used without further purification. Reactions were monitored by thin layer chromatography (TLC). TLC was performed with 0.25 mm E. Merck silica plates (60F-254) using short-wave UV light as the visualizing agent and KMnO4, ninhydrin, vanillin, bromocresol green, iodine and heat as developing agents. LC/HRMS was performed on a Thermo Vanquish UHPLC coupled to an Orbitrap Exploris 120 (HRESI) equipped with an Accucore C18 column (100 mm×2.1 mm), or on an Agilent 1260 Infinity system equipped with Poroshell 120 EC-C18 column (4.6×50 mm, 2.7 μm) and an Agilent G6230B TOF LC/MS with water and acetonitrile buffered with 0.1% formic acid as mobile phases. LCMS analysis was performed on an Agilent 1260 Infinity System coupled to an Agilent 6100 Series Single Quadrupole LCMS equipped with Poroshell 120 EC-C18 column (3.0×50 mm, 2.7 μm). Preparative HPLC purification was performed on an Agilent 1260 Infinity system equipped with a Zorbax Eclipse XDB-C18 PrepHT column (21.2×250 mm, 7 μm) with water and acetonitrile buffered with 0.1% formic acid as mobile phases. NMR spectra were recorded on a Bruker AVANCE AV400 (400 MHz and 101 MHz) or Bruker AVANCE AV600 (600 MHz and 151 MHz) at 23° C. unless otherwise noted. Optical rotations were measured on Autopol IV polarimeter (Rudolph Research Analytical). Sonication was performed using a Qsonica Q500 sonicator. Biochemicals and media components were purchased from standard commercial sources. Expression vectors were obtained via DNA synthesis from Twist Bioscience and were used directly with electrocompetent E. coli BL21(DE3). Electrocompetent E. coli BL21(DE3) strains were purchased from Lucigen. All E. coli strains generated in this work are stored as glycerol stocks at −80° C.

### Proteasome Inhibition Screening

RPMI 8226 cells were seeded in a 6-well plate (1,000,000 cells/well) in cell culture media supplemented with 30 nM of indicated compounds (1,000× stock in DMSO) or DMSO and cultured for 6 hours. Cells were then collected and lysed. Lysates (1 mg/mL, 25 μL) were dispensed into 100 μL assay buffer containing 100 M proteasome substrate Suc-LLVY-AMC (β5) or Ac-RLR-AMC (β2). The plates were incubated at 37° C. for one hour and fluorescence was read at A360ex/A460em. (n=3)

### RPMI8226 Cytotoxicity Screening

RPMI 8226 cells were seeded in a 96-well plate (20,000 cells/well) in cell culture media supplemented with 10 or 30 nM of indicated compounds (1,000× stock in DMSO) or DMSO and cultured for 48 hours. Toxicity was determined using the WST-1 assay (Roche) according to the protocol of the manufacturer. (n=3)

### RPMI 8226 Cytotoxicity EC₅₀ Measurements

RPMI 8226 cells were seeded in a 96-well plate (20,000 cells/well) in cell culture media supplemented with indicated compounds (1,000× stock in DMSO) or DMSO and cultured for 48 hours. Toxicity was determined using the WST-1 assay (Roche) according to the protocol of the manufacturer. (n=3)

### Proteasome Inhibition IC₅₀ Measurements

RPMI 8226 cells were seeded in a 6-well plate (1,000,000 cells/well) in cell culture media supplemented with indicated compounds (1,000× stock in DMSO) or DMSO and cultured for 6 hours. Cells were then collected and lysed. Lysates (1 mg/mL, 25 L) were dispensed into 100 μL assay buffer containing 100 M proteasome substrate Suc-LLVY-AMC (β5) or Ac-RLR-AMC (β2). The plates were incubated at 37° C. for one hour and fluorescence was read at A₃₆₀ₑₓ/A₄₆₀ₑₘ. (n=3)

### MM and MCL Cytotoxicity EC₅₀ Measurements

Cells were seeded in a 96-well plate (20,000 cells/well) in cell culture media supplemented with indicated compounds (1,000× stock in DMSO) or DMSO and cultured for 48 hours. Toxicity was determined using the WST-1 assay (Roche) according to the protocol of the manufacturer. (n=3)

Protein and DNA Sequences
Protein sequence of KDO3
(UniProt accession code: A5FF23)
MKSQSLIEDEIPVKENYAYQIPTSPLIVEVTPQERNILSNVGALL
EKAFKSYENPDYIEALHLYSFQLLPERIARILSRFGTDFSADQYG
AIIFRGLLEVDQDHLGPTPANWQSADYSKLNKYGFICSLLHGAVP
SKPVQYYAQRKGGGILHAVIPDEKMAATQTGSGSKTNLYVHTEDA
FLLHQADFLSFLYLRNEERVPSTLYSVRSHGKVNKIMEKLFDPIY
QCPKDANYQEEINDGPLASVLYGNKKLPFIRFDAAEQIFNENAGQ
TPEALYNLTEFWNEAKELINSDYIPDSGDVIFVNNHLCAHGRSAF
TAGQKEENGKLVPCERRQMLRMMSKTSLIHIRSMTHTDDPYFVME
EHLGKVFDQA
DNA sequence of KDO3
(codon optimized by Genscript)
AAGTCTCAGAGCTTAATTGAAGACGAAATCCCGGTGAAGGAGAAT
TATGCATACCAGATTCCTACGTCTCCTCTCATTGTCGAAGTGACG
CCACAAGAACGCAATATCCTGTCAAATGTAGGTGCGCTCTTGGAG
AAGGCGTTTAAATCGTACGAGAATCCTGATTATATCGAGGCCCTT
CACCTCTACTCATTCCAATTGTTGCCGGAGCGCATCGCGCGTATT
CTCTCTCGTTTCGGCACGGATTTCTCCGCTGATCAGTACGGCGCA
ATCATTTTCCGCGGCCTGCTTGAGGTCGATCAGGACCACTTAGGC
CCGACCCCCGCGAACTGGCAAAGCGCTGACTACAGCAAACTGAAT
AAGTATGGTTTCATCTGTAGCCTCTTACACGGTGCCGTGCCAAGT
AAACCCGTGCAATACTACGCACAACGCAAGGGCGGCGGTATCCTG
CATGCGGTGATTCCGGACGAGAAGATGGCAGCTACCCAAACTGGG
TCCGGAAGTAAAACTAATCTGTACGTTCATACGGAAGATGCCTTC
CTGCTGCATCAAGCCGACTTCTTAAGCTTTCTTTATCTGCGCAAC
GAAGAACGCGTGCCTTCAACATTGTATTCGGTTCGCTCTCACGGT
AAGGTAAACAAGATTATGGAGAAGTTGTTCGACCCGATTTATCAA
TGTCCTAAGGATGCGAACTACCAAGAGGAAATCAACGACGGCCCG
CTGGCCTCAGTTCTGTATGGTAATAAGAAGTTGCCCTTCATTCGC
TTCGATGCCGCGGAACAGATCTTTAACGAGAACGCAGGTCAGACC
CCGGAAGCCTTGTACAACTTAACAGAGTTCTGGAATGAGGCGAAA
GAGTTGATCAACAGTGACTATATCCCTGACAGTGGGGACGTCATT
TTCGTTAATAACCACCTCTGCGCACATGGCCGCTCGGCTTTCACC
GCTGGTCAGAAAGAGGAAAATGGTAAGCTGGTCCCATGTGAGCGC
CGTCAAATGTTACGTATGATGAGCAAGACGAGTTTGATCCATATT
CGCAGTATGACTCACACTGATGACCCTTATTTTGTTATGGAAGAG
CACTTGGGAAAGGTCTTTGATCAAGCCTAA

Expression vectors for KDO1^{S1} and SadA/LasA^{S2} were constructed as described in previously reported procedures. For the construction of expression vectors, each of the above sequences was inserted between the NdeI and BamHI restriction sites within the commercial pET28a(+) vector. The resulting expression vector was used directly to transform electrocompetent E. coli strain BL21(DE3). Variants were stored as glycerol stocks at −80° C.

Hybrid analog (S39) EC₅₀ determination

Cytotoxicity of phenyl cepafungin (50) and hybrid analog (S39) in RPMI 8226 RPMI 8226 cells were seeded in a 96-well plate (20,000 cells/well) in cell culture media supplemented with indicated compounds (1,000× stock in DMSO) or DMSO and cultured for 48 hours. Toxicity was determined using the WST-1 assay (Roche) according to the protocol of the manufacturer. (n=3)

### Synthetic Procedures

### KDO3 Reaction Optimization:

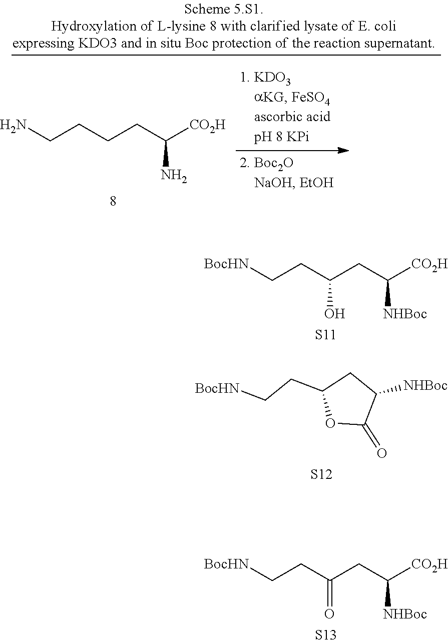

### Standard Conditions:

A glycerol stock of E. coli BL21(DE3) cells harboring pET-28a(+)-KDO3 plasmid was used to inoculate an overnight culture of LB media (4 mL) containing 50 μg/mL kanamycin. 0.5 mL of this culture was used to inoculate 100 mL TB media containing 50 μg/mL kanamycin in a 500 mL non-beveled Erlenmeyer flask. The culture was shaken at 250 rpm at 37° C. for 2.5 h or until an OD₆₀₀=0.6 was reached. The culture was cooled on ice (15 min), induced by adding IPTG to final concentration of 25 μM, then allowed to continue shaking at 250 rpm/22° C. for another 22 h. Cells were harvested by centrifugation (4° C., 15 min, 4200 rpm), then resuspended in 50 mM pH=8 KPi buffer (ca. 25 mL) to a final OD₆₀₀=30. The cell suspension was lysed by sonication at 50% amplitude for 3 min (1 s on, 4 s off) in an ice bath. The cell debris was pelleted by centrifugation (4° C., 15 min, 4200 rpm), and the clarified lysate supernatant was diluted with 50 mM pH=8 KPi and added to a non-beveled Erlenmeyer flask (≥80% headspace) containing L-lysine (1 equiv), α-ketoglutaric acid (1.25 equiv), ascorbic acid (0.5 equiv) and FeSO₄·7H₂O (0.1 equiv). The reaction mixture was then shaken for 10 h at 200 rpm/23° C. in the open Erlenmeyer flask. The reaction was then quenched by addition of 6 M HCl to final pH=2 and centrifuged (4° C., 15 min, 4200 RPM). The supernatant was directly subjected to the next reaction.

The supernatant was basified with 6 M NaOH to pH=11. With rapid stirring, Boc₂O (4 equiv) dissolved in EtOH (final 2:1 H₂O:EtOH) was slowly added at rt. After 30 min, the mixture was adjusted back to pH=10 with additional 6 M NaOH and allowed to stir overnight. The mixture was again adjusted back to pH=10 with 6 M NaOH and Boc₂O (2 equiv) dissolved in minimal EtOH was slowly added at rt. The mixture was allowed to stir overnight or until completion as judged by LCMS. Ethanol was removed from the reaction mixture in vacuo. The aqueous phase was adjusted to pH=1 with 6 M HCl and diluted with 75 mL EtOAc. The aqueous layer was separated and extracted with an additional 3×50 mL EtOAc. The combined organic layers were washed with brine and dried over Na₂SO₄, filtered, and concentrated. The crude material was purified by flash chromatography (97:3:1 DCM:MeOH:AcOH) followed by two evaporations from toluene to provide compounds S11-13 as white solids.

TABLE 5.S1
KDO3 reaction optimization. Clarified lysates generated from 100
mL cell culture at pre-lysis OD₆₀₀ = 30 were diluted with varying
volumetric equivalents of 50 mM pH 8 KPi and reacted with varying
final concentrations of L-lysine substrate. KDO3 reactions in entries
1, 2 and 5-8 were performed under standard conditions. Co-substrate
ratios and buffer conditions in entries 3 and 4 (products not isolated)
are adapted from the procedure reported by Baud.^{S4}

| | | | | | | Yield |
|---|---|---|---|---|---|---|
| | | NMR ratio | Yield | Yield | Yield | (S11 + |
| Entry | Conditions^{a} | S11:S13 | S11^{b} | S12^{b} | S13^{b} | S12)^{b} |
| 1 | 20 mM Lys, lysate | 1.0:2.9 | 11% | 6% | 32% | 17% |
| | diluted 2x | | | | | |
| 2 | 40 mM Lys, lysate | 1.1:1.0 | 32% | 7% | 17% | 39% |
| | diluted 2x | | | | | |
| 3 | 20 mM Lys, 1.5 eq | Only | — | — | — | — |
| | aKG, 0.25 eq | S13 + | | | | |
| | ascorbate, 0.1 eq | byproducts | | | | |
| | FeSO₄. pH 7.5 | | | | | |
| | HEPES. Lysate | | | | | |
| | diluted 1x | | | | | |
| 4 | 40 mM Lys, 1.5 eq | 0.4:1.0 | — | — | — | — |
| | aKG, 0.25 eq | | | | | |
| | ascorbate, 0.1 eq | | | | | |
| | FeSO₄. pH 7.5 | | | | | |
| | HEPES. Lysate | | | | | |
| | diluted 1x | | | | | |
| 5 | 40 mM Lys, lysate | 1.0:1.3 | 24% | 17% | 19% | 41% |
| | diluted 3x | | | | | |
| 6 | 40 mM Lys, lysate | 1.0:0.9 | 5% | 30% | 17% | 35% |
| | diluted 4x | | | | | |
| 7 | 40 mM Lys, lysate | 1.0:0.8 | 17% | 22% | 14% | 39% |
| | diluted 5x | | | | | |
| 8 | 40 mM Lys (gram | 1.0:0.5 | 20% | 22% | 12% | 42% |
| | scale), lysate diluted | | | | | |
| | 6x | | | | | |

^{a}Standard conditions: (1) 1.25 eq. αKG, 0.1 eq. FeSO₄•7H₂O, 0.5 eq. ascorbic acid, 50 mM pH 8 KPi, 200 RPM/23° C., 10 h; HCl quench. (2) 6 eq. Boc₂O, NaOH to pH = 11, 2:1 H₂O:EtOH
^{b}Isolated yield after purification by flash chromatography as described above

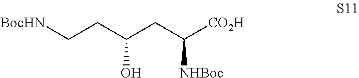 [SMILES: CC(C)(C)OC(=O)NCC[C@@H](O)C[C@H](NC(=O)OC(C)(C)C)C(=O)O]

(2S,4R)-2,6-bis((tert-butoxycarbonyl)amino)-4-hydroxyhexanoic acid (S11). Prepared on gram-scale following standard conditions in entry 8, Table 5.S1. L-lysine 8 (1.02 g, 7 mmol, 1 equiv), α-ketoglutaric acid (1.98 g, 8.75 mmol, 1.25 equiv), ascorbic acid (616 mg, 3.5 mmol, 0.5 equiv), Fe₂SO₄·7H₂O (195 mg, 0.7 mmol, 0.1 equiv) and Boc₂O (9.17 g, 10.5 mmol, 6 equiv) were used to afford S11 (500 mg, 20% over 2 steps) as a white solid.

¹H NMR (400 MHz, Methanol-d₄): δ 4.33-4.13 (m, 1H), 3.78-3.65 (m, 1H), 3.15 (td, J=6.8, 2.7 Hz, 2H), 1.91-1.79 (m, 1H), 1.79-1.67 (m, 1H), 1.65-1.53 (m, 2H), 1.44 (d, J=3.9 Hz, 18H).

¹³C NMR (151 MHz, Methanol-d₄): δ 176.6, 158.8, 158.4, 80.5, 80.0, 66.5, 52.3, 39.8, 39.0, 38.1, 28.8, 28.7.

HRMS (ESI): calculated for C₁₆H₃₁N₂O₇⁺ ([M+H]⁺) 363.2126, found 363.2125.

[α]_{D}²⁴=−3.8(c0.7,MeOH)

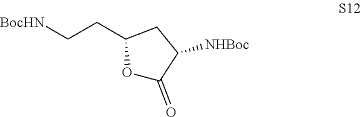 [SMILES: CC(C)(C)OC(=O)NCC[C@@H]1C[C@H](NC(=O)OC(C)(C)C)C(=O)O1]

tert-butyl (2-((2R,4S)-4-((tert-butoxycarbonyl)amino)-5-oxotetrahydrofuran-2-yl)ethyl)carbamate (S12). Prepared on gram-scale following standard conditions in entry 8, Table 5.S1. L-lysine 8 (1.02 g, 7 mmol, 1 equiv), α-ketoglutaric acid (1.98 g, 8.75 mmol, 1.25 equiv), ascorbic acid (616 mg, 3.5 mmol, 0.5 equiv), Fe₂SO₄·7H₂O (195 mg, 0.7 mmol, 0.1 equiv) and Boc₂O (9.17 g, 10.5 mmol, 6 equiv) were used to afford S12 (515 mg, 22% over 2 steps) as a white solid.

¹H NMR (400 MHz, Methanol-d₄): δ 4.54-4.24 (m, 2H), 3.28-3.12 (m, 2H), 2.61 (ddd, J=12.1, 8.8, 5.5 Hz, 1H), 2.03-1.78 (m, 3H), 1.44 (d, J=6.2 Hz, 18H).

¹³C NMR (151 MHz, Methanol-d₄): δ 177.2, 158.5, 157.6, 80.8, 80.1, 77.0, 52.2, 37.7, 36.6, 35.8, 28.8, 28.7.

HRMS (ESI): calculated for C₁₆H₂₉N₂O₆ ([M+H]⁺) 345.2020, found 345.2015.

[α]_{D}²⁷=+12.7(c0.5,MeOH)

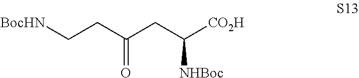 [SMILES: CC(C)(C)OC(=O)NCCC(=O)C[C@H](NC(=O)OC(C)(C)C)C(=O)O]

(S)-2,6-bis((tert-butoxycarbonyl)amino)-4-oxohexanoic acid (S13). Prepared on gram-scale following standard conditions in entry 8, Table 5.S1. L-lysine 8 (1.02 g, 7 mmol, 1 equiv), α-ketoglutaric acid (1.98 g, 8.75 mmol, 1.25 equiv), ascorbic acid (616 mg, 3.5 mmol, 0.5 equiv), Fe₂SO₄·7H₂O (195 mg, 0.7 mmol, 0.1 equiv) and Boc₂O (9.17 g, 10.5 mmol, 6 equiv) were used to afford S13 (292 mg, 12% over 2 steps) as a white solid.

¹H NMR (600 MHz, Methanol-d₄): δ 4.53-4.37 (m, 1H), 3.28 (t, J=6.5 Hz, 2H), 3.02-2.84 (m, 2H), 2.74-2.58 (m, 2H), 1.43 (d, J=10.2 Hz, 18H).

¹³C NMR (151 MHz, Methanol-d₄): δ 208.16, 175.09, 158.35, 157.83, 80.65, 80.13, 50.60, 45.08, 43.62, 36.29, 28.75, 28.70.

HRMS (ESI): calculated for C₁₆H₂₉N₂O₇⁺ ([M+H]⁺) 361.1969, found 361.1967.

[α]_{D}²⁴=−2.2(c1.0,MeOH)

### General Procedures for Analog Syntheses

### General Procedure A: Aminolysis of 4-Hydroxylysine Lactones

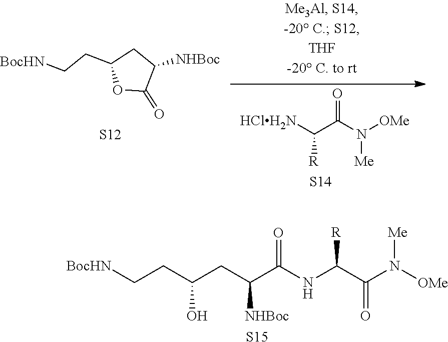

Amine hydrochloride S14 (5 equiv) was set stirring in anhydrous THF (0.4 M). The mixture was cooled to −20° C., then a 2 M solution of trimethylaluminum (4.95 equiv) in anhydrous toluene was added dropwise. The reaction was stirred under argon at −20° C. for 30 minutes to provide a clear, light-yellow solution. Lactone S12 (0.4 M, 1 equiv) in anhydrous THF was added dropwise at −20° C. The reaction was stirred to room temperature over 28 h or until completion by TLC. The reaction was cooled to 0° C. and quenched by dropwise addition of 2 M NaHSO₄ solution. The mixture was adjusted to pH=1 with 1 M HCl, then concentrated in vacuo to remove THF. The aqueous phase was extracted three times with ethyl acetate. The combined organic layers were then washed with H₂O, NaHCO₃, H₂O, brine and dried over Na₂SO₄, filtered and concentrated. The residue was purified by flash chromatography using a step gradient of hexanes/EtOAc/MeOH mixture as eluent to provide S15 as a white solid.

### General Procedure B: Reduction of Weinreb Amides

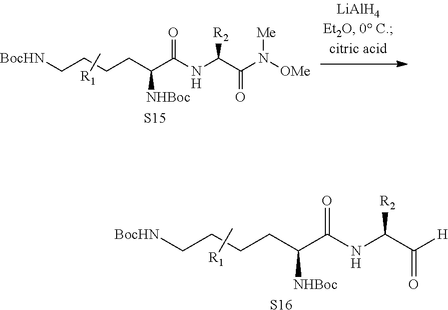

Weinreb amide S15 (1 equiv, 0.1 M) was set stirring in anhydrous Et₂O and the solution was cooled to 0° C. 1 M LiAlH₄ solution (2.5 equiv) in Et₂O was added dropwise. The mixture was stirred at 0° C. under argon for 30 minutes or until completion by TLC, then quenched at 0° C. by dropwise addition of 2 M citric acid (2.5 equiv). The mixture was diluted with 10% v/v H₂O and stirred for 5 min. The mixture was diluted with additional H₂O and Et₂O, then the layers were separated. The aqueous layer was extracted three times with Et₂O, and the combined organic layers were washed with H₂O, NaHCO₃, brine and dried over Na₂SO₄, filtered and concentrated to provide S16 as white solid (d.r.≥98:2) used immediately without further purification.

### General Procedure C: Wittig Olefination

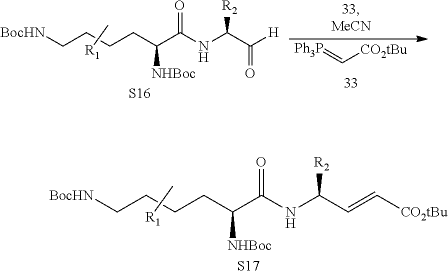

Ylide 33 (1.25 equiv) was dissolved in freshly-distilled anhydrous acetonitrile (0.2 M), then a solution of aldehyde S16 (1 equiv) in acetonitrile (0.2 M) was added dropwise at room temperature. The reaction was stirred for 30 minutes or until completion by TLC, and then concentrated to dryness. Purification by silica flash chromatography using a mixture of DCM/EtOAc/MeOH as eluent provided S17 as an off-white solid.

### General Procedure D: Global Deprotection

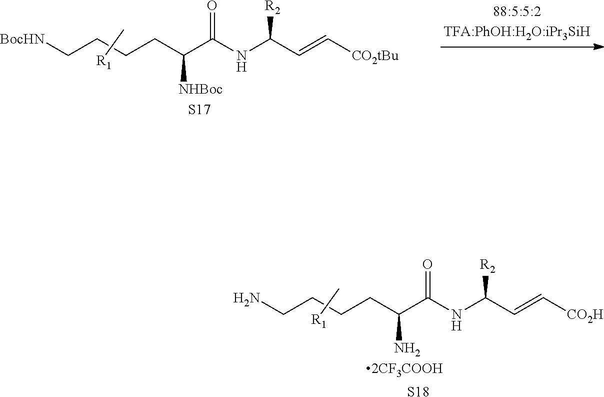

Dipeptide S17 (1 equiv) was dissolved (0.04 M) in a freshly prepared solution of “Reagent B” (88:5:5:2 TFA:PhOH:H₂O:iPr₃SiH) and the mixture was stirred for 1 hour at rt or until completion by LCMS. The reaction was diluted with toluene and concentrated in vacuo to dryness, then evaporated twice more from 30 mL toluene. The residue was dissolved in minimal MeOH and triturated into diethyl ether at 0° C. The solids were collected by centrifugation (4° C., 10 min, 4200 rpm), then triturated into ether and centrifuged as above once more, then dried from MeOH to provide pure S18 as an off-white solid.

### General Procedure E: Macrolactamization

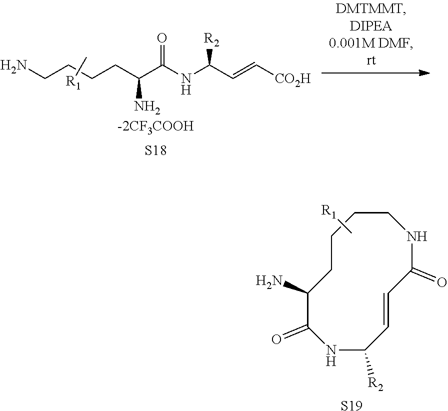

Linear macrocycle precursor S18 (1 equiv) was dissolved anhydrous DMF (0.001 M) and treated with diisopropylethylamine (4 equiv) then 4-(4,6-dimethoxy-1,3,5-triazin-2-yl)-4-methyl-morpholinium tetrafluoroborate (DMTMMT) (1.50 equiv) at rt. The reaction was stirred under argon for 36 hours, then quenched with 10% v/v H₂O and concentrated in vacuo to dryness. The residue was dissolved in minimal methanol and triturated into diethyl ether with rapid stirring. The solids were collected by centrifugation (4° C., 15 min, 4200 rpm) and dried from MeOH to provide S19 as a tan solid. Yield was determined by ¹H NMR analysis with 4-toluenesulfonamide as internal standard.

### General Procedure F: Fragment Coupling

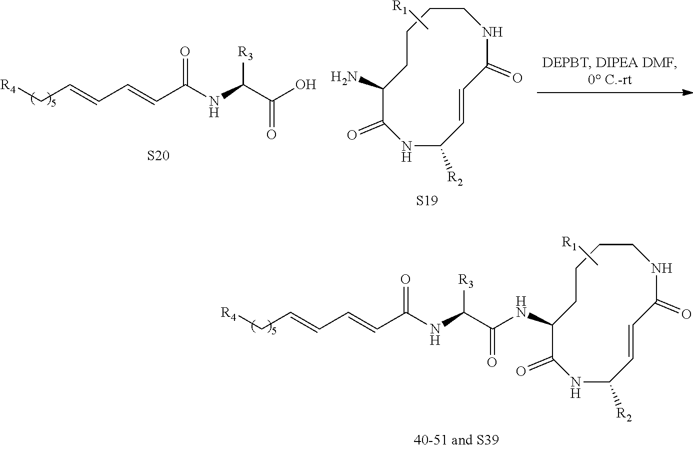

Carboxylic acid S20 (2 equiv.) and crude macrocycle S19 (1 equiv as judged by ¹H NMR analysis) were dissolved in anhydrous DMF (0.08 M) and cooled to 0° C. To the mixture was added diisopropylethylamine (6 equiv), followed by DEPBT (2 equiv). The reaction was warmed to room temperature and stirred overnight or until completion by LCMS. The reaction was quenched at 0° C. 30% v/v H₂O then concentrated in vacuo to dryness. The residue was triturated into 50 mL Et₂O at 0° C. from minimal MeOH, and the resulting solids were collected by centrifugation (4° C., 15 min, 4200 rpm). The solids were dissolved in DMSO, filtered through a 0.2 mm PTFE membrane filter, then purified by preparative reversed-phase HPLC using a gradient of MeCN/H₂O/0.1% formic acid mixtures over 35 minutes to provide 40-51 and S39 as fluffy white powders after lyophilization.

### General Procedure G: Kochi Coupling

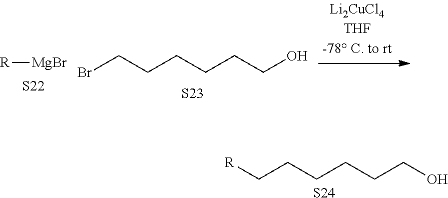

Following a procedure adapted from Melaugh et al.,^{S5} lithium chloride (2.05 equiv) was flame-dried in a round-bottom flask under high vacuum and purged with argon (3×). Solid copper(II) chloride (1 equiv) was added under argon. The combined reagents were then dissolved in anhydrous THF (0.7 M with respect to CuCl₂) with rapid stirring to provide a dark red solution of Li₂CuCl₄.

Grignard reagent S22 was used as provided from commercial sources or prepared as described previously.^{S6} A flame-dried flask was charged with S22 (6.5 equiv) in anhydrous THF (1 M). The mixture was cooled to −78° C. and S23 (1 equiv) was added in anhydrous THF (7 M), followed by the above solution of Li₂CuCl (1.1 equiv with respect to S24). Upon complete addition, the reaction was removed from its cooling bath and allowed to warm to room temperature. Reaction progress was monitored by TLC until completion at 1 h. The reaction was then carefully quenched at 0° C. with saturated NH₄Cl and filtered through a sintered glass funnel. The filtrate was concentrated in vacuo to remove most of the THF, then diluted with ethyl acetate and 1 M HCl to a final pH=1. The layers were separated, and the aqueous phase was extracted three times with ethyl acetate. The combined organic layers were washed with H₂O, NaHCO₃ and brine, then dried over Na₂SO₄, filtered and concentrated. Purification by silica flash chromatography using hexanes/EtOAc as eluent provided pure S24 as a colorless liquid.

### General Procedure H: Swern Oxidation

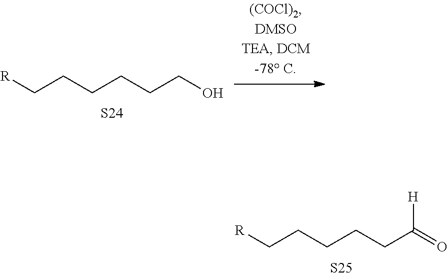

Oxalyl chloride (1.5 equiv) was dissolved in anhydrous DCM (1.3 M) at −78° C. A solution of anhydrous DMSO (3 equiv) in anhydrous DCM (7 M) was added dropwise and the resulting mixture was stirred at −78° C. for 15 minutes. A solution of S24 (1 equiv) in anhydrous DCM (1 M) was then added dropwise over 10 minutes. The mixture was stirred at −78° C. for 1 hour then treated with triethylamine (5 equiv) dropwise. The resulting thick slurry was diluted with additional DCM to aid stirring, and reaction progress was monitored by TLC until completion at 1 hour. The reaction was quenched at −78° C. with H₂O and allowed to stir to room temperature. The layers were separated, and the aqueous layer was extracted three times with DCM. The combined organic layers were washed with H₂O, brine, then dried over Na₂SO₄ and filtered through a plug of silica with ether as eluent. Concentration in vacuo provided S25 as a colorless oil which was used immediately in the next reaction (General Procedure I) without further purification.

### General Procedure I. Allylic HWE

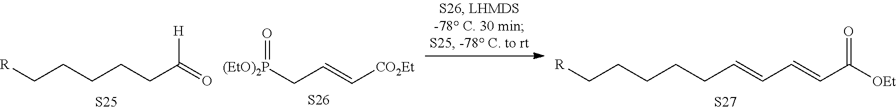

In a procedure adapted from Imker et al.,^{S7} S26 (1.5 equiv) in anhydrous THF (0.5 M) was cooled to −78° C. and treated with lithium hexamethyldisilazide (1 M THF, 1.4 equiv) dropwise, then allowed to stir under argon at −78° C. for 30 min. Aldehyde S25 (1 equiv) in anhydrous THF (1.4 M) was added dropwise at −78° C. and the mixture was allowed to stir to room temperature overnight or until completion by TLC. The reaction was quenched at 0° C. with saturated NH₄Cl solution. The layers were separated and the aqueous layer was extracted three times with ethyl acetate. The combined organic layers were washed with H₂O, NaHCO₃, brine and dried over Na₂SO₄, filtered and concentrated. Purification by silica flash chromatography using hexanes/Et₂O as eluent provided S27 as a light yellow oil.

### General Procedure J: Ester Hydrolysis

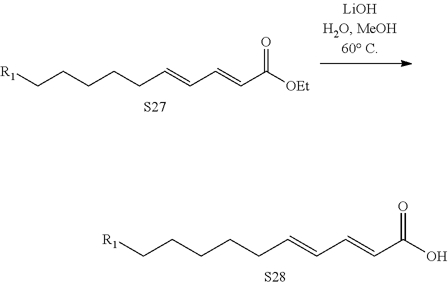

Ester S27 (1 equiv) was dissolved in MeOH (0.2 M). With rapid stirring, an aqueous solution of lithium hydroxide (1 M, 5 equiv) was added, and the reaction was heated to 60° C. for 5 hours or until completion by TLC. Methanol was evaporated in vacuo, then the mixture was adjusted to pH 1 with 1 M HCl and diluted with 20 mL DCM. The layers were separated, and the aqueous layer was extracted three times with DCM. The combined organic layers were washed with brine, dried over Na₂SO₄, filtered and concentrated to provide pure S28 as a waxy white solid.

### General Procedure K: Amide Coupling

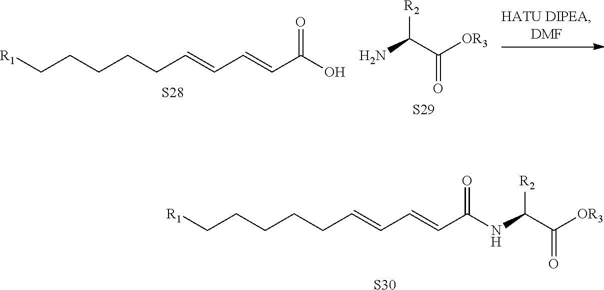

Fatty acid S28 (1 equiv) was combined with amine S29 (1-1.5 equiv) in anhydrous DMF (0.2 M). Diisopropylethylamine was added (1-3 eq), followed by HATU (1.05 equiv). The reaction was stirred at room temperature under argon for 4 hours or until completion by LCMS. The reaction was then quenched with 10% v/v H₂O and concentrated in vacuo. The residue was dissolved in ethyl acetate and washed with 1 M HCl (2×), H₂O, NaHCO₃, and brine, then dried over Na₂SO₄, filtered and concentrated. Purification by flash chromatography using hexanes:EtOAc as eluent provided S30 as a colorless oil.

### General Procedure L: Tail Fragment Deprotection

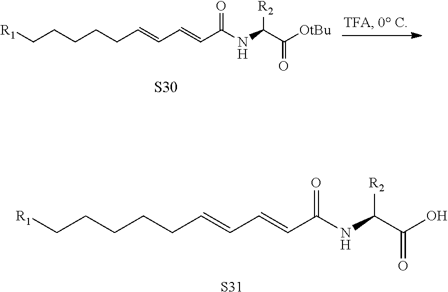

Amide S30 (1 equiv) was dissolved in TFA (0.1 M) at 0° C. and stirred for 5 min, then stirred at rt for 1 hour. The reaction was then diluted with toluene and concentrated in vacuo to dryness. The residue was evaporated twice more from toluene to provide S31 as a pale yellow resin.

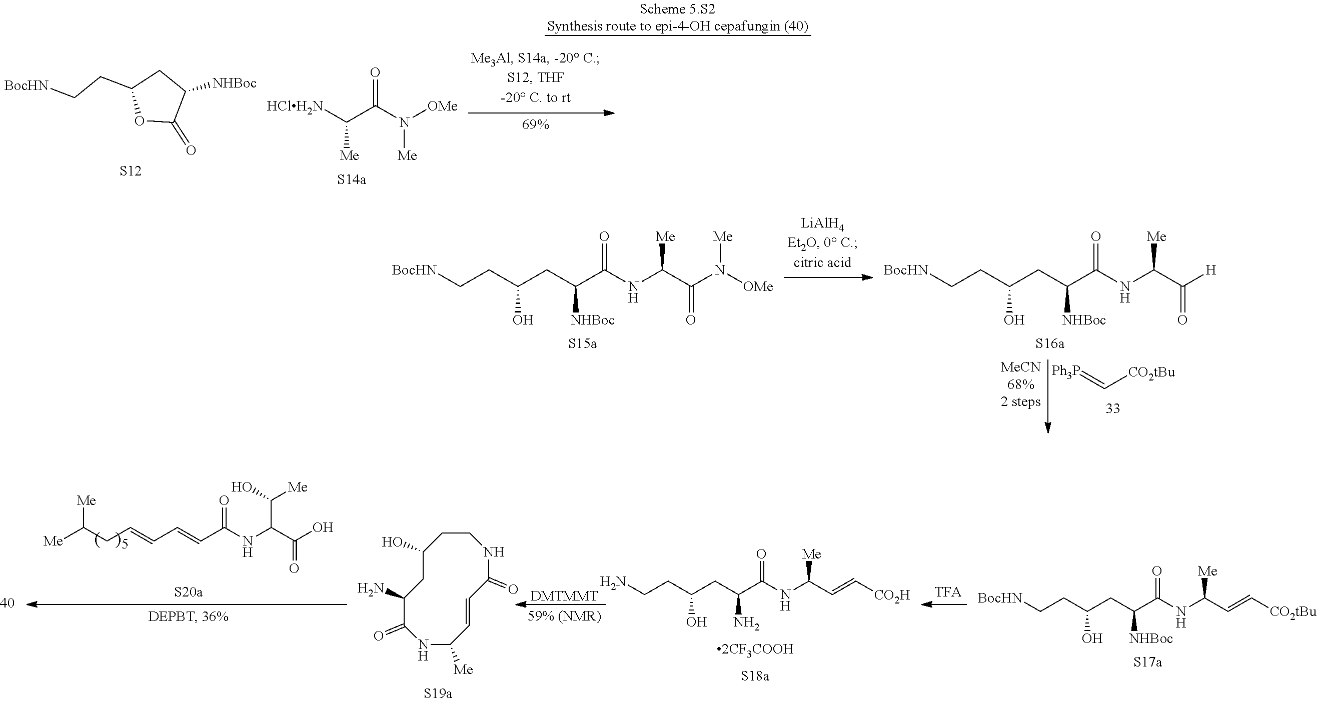

### di-tert-butyl ((3R,5S)-3-hydroxy-6-(((S)-1-(methoxy(methyl)amino)-1-oxopropan-2-yl)amino)-6-oxohexane-1,5-diyl)dicarbamate (S15a)

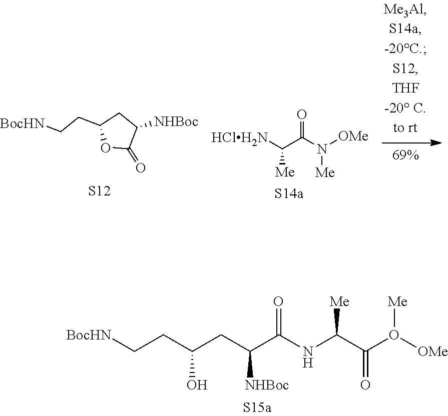

Lactone S12 was prepared as described above in Table 5.S1, entry 8. Amine S14a was prepared as described in a previous report.^{S6} Following General Procedure A: S12 (638 mg, 1.85 mmol, 1 equiv), S14a (1.56 g, 9.26 mmol, 5 equiv) and Me₃Al (4.58 mL 2 M toluene, 9.16 mmol, 4.95 equiv) in THE (28 mL) were used to produce S15a (609 mg, 69%) as a white solid. ¹H NMR (600 MHz, CDCl₃): δ 7.10-6.92 (m, 1H), 6.01-5.72 (m, 1H), 4.95-4.84 (m, 1H), 4.36 (q, J=6.3, 5.8 Hz, 1H), 3.76 (s, 4H), 3.41 (qd, J=8.5, 5.5, 4.6 Hz, 1H), 3.20 (s, 3H), 3.10 (dt, J=14.2, 5.4 Hz, 1H), 2.96 (s, 1H), 1.79 (t, J=6.5 Hz, 2H), 1.64-1.49 (m, 2H), 1.46-1.40 (m, 19H), 1.34 (d, J=6.9 Hz, 3H).

¹³C NMR (151 MHz, CDCl₃): δ 172.8, 171.4, 157.0, 156.6, 80.4, 79.6, 66.5, 61.7, 52.3, 46.0, 40.9, 37.8, 37.5, 32.3, 28.5, 28.4, 18.4.

HRMS (ESI): calculated for C₂₁H₄₁N₄O₈⁺ ([M+H]⁺) 477.2919, found 477.2920.

[α]_{D}²⁷=−24.4(c1.4,MeOH)

### di-tert-butyl ((3R,5S)-3-hydroxy-6-oxo-6-(((S)-1-oxopropan-2-yl)amino)hexane-1,5-diyl)dicarbamate (S16a)

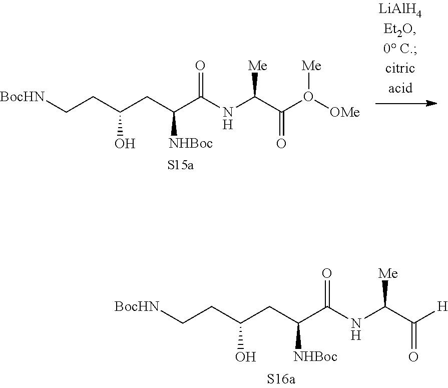

Following General Procedure B: S15a (300 mg, 0.630 mmol, 1 equiv) and LAH (60 mg, 1.57 mmol, 2.5 equiv) in Et₂O (7.9 mL) were used to provide S16a (323 mg) as a white solid.

¹H NMR (400 MHz, CDCl₃): δ 9.53 (s, 1H), 7.24 (d, J=5.0 Hz, 1H), 6.12 (d, J=7.4 Hz, 1H), 4.91 (s, 1H), 4.68 (s, 1H), 4.45-4.34 (m, 2H), 3.84-3.78 (m, 1H), 3.13-3.01 (m, 1H), 1.98-1.88 (m, 1H), 1.87-1.73 (m, 2H), 1.64-1.48 (m, 2H), 1.43 (d, J=7.0 Hz, 18H), 1.35 (d, J=7.4 Hz, 3H).

HRMS (ESI): calculated for C₁₉H₃₆N₃O₇⁺ ([M+H]⁺) 418.2548, found 418.2553.

### tert-butyl (S,E)-4-((2S,4R)-2,6-bis((tert-butoxycarbonyl)amino)-4-hydroxyhexanamido)pent-2-enoate (S17a)

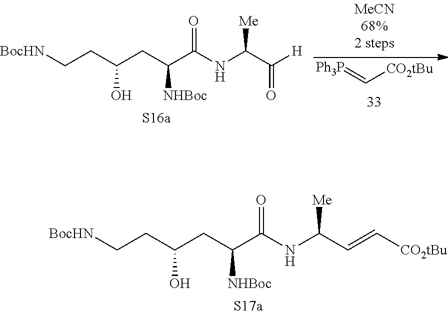

Following General Procedure C: S16a (94 mg, 0.225 mmol, 1 equiv), 33 (106 mg, 0.281 mmol, 1.25 equiv) in MeCN (3.2 mL) were used to provide S17a (80 mg, 68% over 2 steps) as a white solid.

¹H NMR (600 MHz, Methanol-d₄): δ 6.77 (dd, J=15.7, 4.9 Hz, 1H), 5.81 (dd, J=15.7, 1.7 Hz, 1H), 4.63-4.56 (m, 1H), 4.27-4.15 (m, 1H), 3.75-3.66 (m, 1H), 3.15 (td, J=6.8, 4.6 Hz, 2H), 1.79-1.68 (m, 2H), 1.63-1.55 (m, 2H), 1.48-1.42 (m, 27H), 1.28 (d, J=7.1 Hz, 3H).

¹³C NMR (151 MHz, Methanol-d₄): δ 174.88, 167.38, 158.82, 157.96, 149.24, 122.79, 81.72, 80.76, 80.03, 66.53, 53.70, 46.91, 40.42, 39.11, 38.11, 28.83, 28.77, 28.35, 19.91.

HRMS (ESI): calculated for C₂₅H₄₆N₃O₈⁺ ([M+H]⁺) 516.3279, found 516.3289.

[α]_{D}²⁷=−18.2(c1.1,MeOH)

### (S,E)-4-((2S,4R)-2,6-diamino-4-hydroxyhexanamido)pent-2-enoic Acid (S18a)

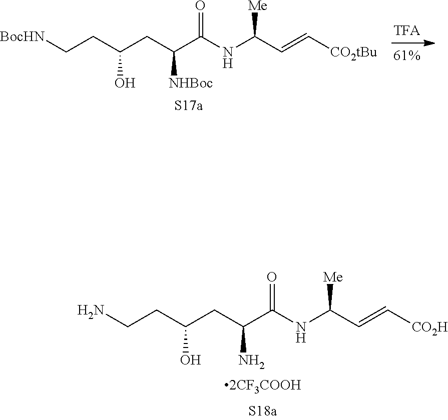 [SMILES: C[C@@H](/C=C/C(=O)OC(C)(C)C)NC(=O)[C@H](C[C@H](O)CCNC(=O)OC(C)(C)C)NC(=O)OC(C)(C)C ; C[C@@H](/C=C/C(=O)O)NC(=O)[C@@H](N)C[C@H](O)CCN]

Following General Procedure D: S17a (336 mg, 0.652 mmol, 1 equiv) was used to provide S18a (194 mg, 61%) as a white solid.

¹H NMR (400 MHz, Methanol-d₄): δ 6.84 (dd, J=15.7, 5.3 Hz, 1H), 5.86 (dd, J=15.7, 1.6 Hz, 1H), 4.70-4.61 (m, 1H), 4.08 (t, J=5.7 Hz, 1H), 3.94-3.84 (m, 1H), 3.15-3.00 (m, 2H), 2.08-1.98 (m, 2H), 1.92-1.75 (m, 2H), 1.33 (d, J=7.0 Hz, 3H).

¹³C NMR (101 MHz, Methanol-d₄): δ 170.0, 169.3, 149.3, 122.5, 66.9, 52.4, 47.4, 38.7, 38.0, 35.8, 19.7.

HRMS (ESI): calculated for C₁₁H₂₂N₃O₄⁺ ([M+H]⁺) 260.1605, found 260.1606.

[α]_{D}²⁷=−17.4(c0.5,MeOH)

### (5S,8S,10R,E)-8-amino-10-hydroxy-5-methyl-1,6-diazacyclododec-3-ene-2,7-dione (S19a)

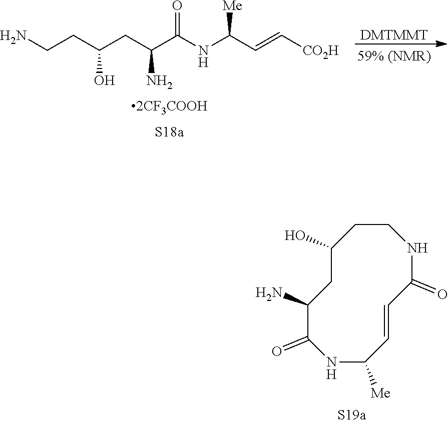

Following General Procedure E: S18a (163 mg, 0.335 mmol, 1 equiv) was used to provide S19a (276 mg, 59%) as a tan solid. Yield was determined by ¹H NMR analysis of a 6.2 mg sample of crude S19a and 5.1 mg 4-toluenesulfonamide added as internal standard. ¹H NMR (400 MHz, Methanol-d₄): 4-toluenesulfonamide: δ 7.85-7.70 (m, 2H), 7.41-7.28 (m, 2H), 2.42 (s, 3H). Compound S19a: δ 7.07 (ddd, J=15.3, 11.8, 4.8 Hz, 1H), 6.29 (ddd, J=15.4, 7.8, 1.3 Hz, 1H), 4.69-4.60 (m, 1H), 4.56 (q, J=6.5 Hz, 1H), 3.49-3.35 (m, 2H), 2.49 (dd, J=16.1, 3.7 Hz, 1H), 2.11-1.96 (m, 1H), 1.87 (ddd, J=15.7, 10.5, 3.4 Hz, 1H), 1.80-1.58 (m, 3H).

HRMS (ESI): calculated for C₁₁H₂₀N₃O₃⁺ ([M+H]⁺) 242.1499, found 242.1495.

### (2E,4E)-N-((2S,3R)-3-hydroxy-1-(((5S,8S,10R,E)-10-hydroxy-5-methyl-2,7-dioxo-1,6-diazacyclododec-3-en-8-yl)amino)-1-oxobutan-2-yl)-11-methyldodeca-2,4-dienamide (40)

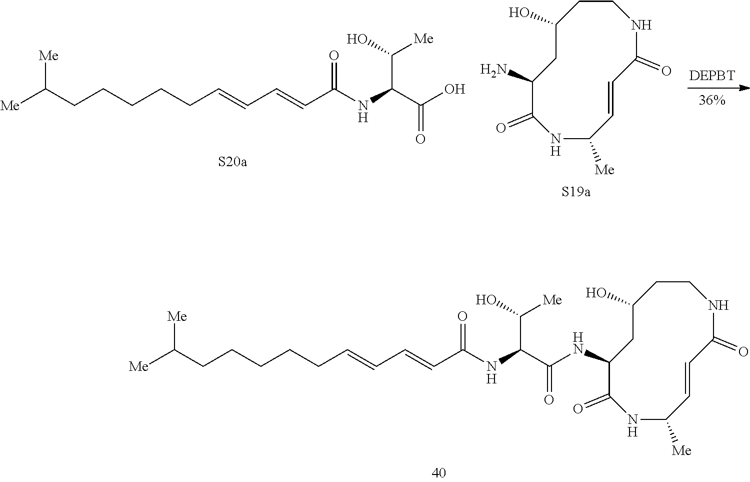 [SMILES: CC(C)CCCCC/C=C/C=C/C(=O)N[C@H](C(=O)O)[C@@H](C)O ; C[C@H]1/C=C/C(=O)NCC[C@@H](O)C[C@H](N)C(=O)N1 ; CC(C)CCCCC/C=C/C=C/C(=O)N[C@H](C(=O)N[C@H]1C[C@H](O)CCNC(=O)/C=C/[C@H](C)NC1=O)[C@@H](C)O]

Following General Procedure F: Carboxylic acid S20a was synthesized as described previously.² S20a (38 mg, 0.121 mmol, 2 equiv), S19a (85 mg, 0.0604 mmol by ¹H NMR analysis, 1 equiv), DEPBT (36 mg, 0.121 mmol, 2 equiv) and DIPEA (63 μL, 0.362 mmol, 6 equiv) were used to provide 40 as a fluffy white solid (11.6 mg, 36%).

¹H NMR (600 MHz, DMSO-d₆): δ 8.42 (d, J=7.8 Hz, 1H), 8.07 (d, J=7.7 Hz, 1H), 7.92 (d, J=8.6 Hz, 1H), 7.42 (dd, J=8.0, 6.1 Hz, 1H), 6.99 (dd, J=15.1, 10.7 Hz, 1H), 6.79 (dd, J=15.3, 4.7 Hz, 1H), 6.22-6.04 (m, 4H), 4.97 (d, J=3.3 Hz, 1H), 4.84 (d, J=5.7 Hz, 1H), 4.48 (dt, J=7.7, 3.8 Hz, 1H), 4.38 (tdd, J=12.3, 6.3, 3.8 Hz, 1H), 4.26 (dd, J=8.6, 4.0 Hz, 1H), 4.03-3.93 (m, 1H), 3.55-3.47 (m, 1H), 3.20-3.10 (m, 1H), 3.02-2.92 (m, 1H), 2.43-2.33 (m, 1H), 2.12 (q, J=7.2 Hz, 2H), 1.58-1.47 (m, 3H), 1.47-1.41 (m, 1H), 1.41-1.35 (m, 2H), 1.28-1.22 (m, 4H), 1.20 (d, J=7.1 Hz, 3H), 1.16-1.11 (m, 2H), 1.03 (d, J=6.3 Hz, 3H), 0.84 (d, J=6.6 Hz, 6H).

¹³C NMR (151 MHz, DMSO-d₆): δ 170.9, 169.3, 165.5, 165.4, 147.3, 142.0, 139.7, 128.6, 123.0, 118.3, 66.5, 64.4, 58.2, 51.8, 45.7, 41.1, 38.4, 37.9, 34.8, 32.2, 28.9, 28.4, 27.4, 26.6, 22.5, 19.4, 18.3.

HRMS (ESI): calculated for C₂₈H₄₇N₄O₆⁺ ([M+H]⁺) 535.3490, found 535.3474.

[α]_{D}²⁷=−51.5(c0.1,MeOH)

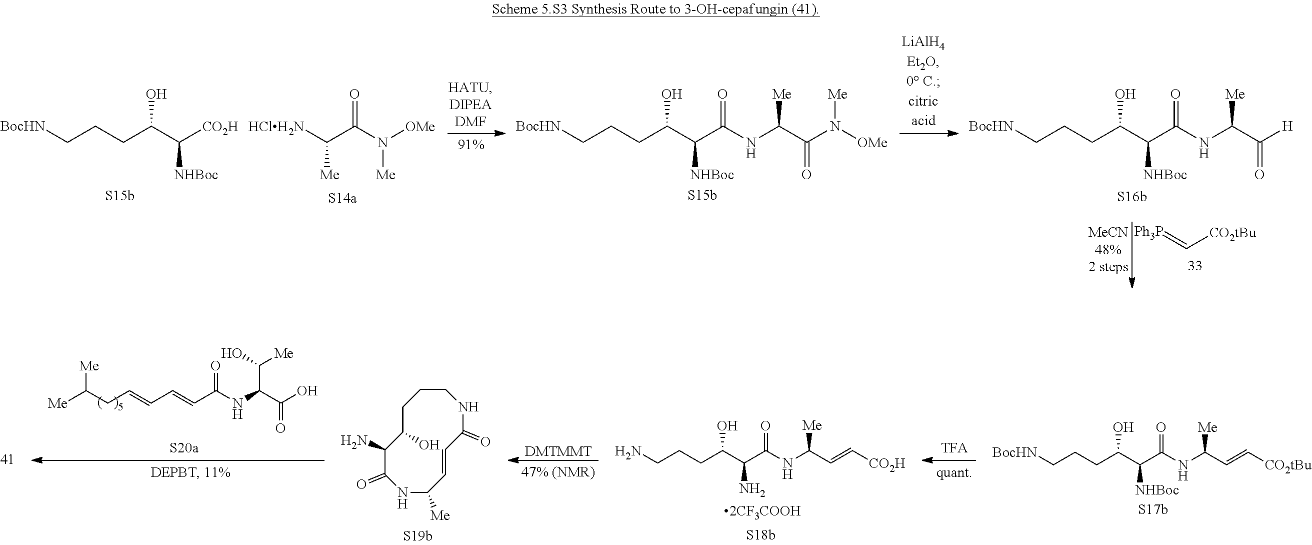

### di-tert-butyl ((4S,5S)-4-hydroxy-6-(((S)-1-(methoxy(methyl)amino)-1-oxopropan-2-yl)amino)-6-oxohexane-1,5-diyl)dicarbamate (S15b)

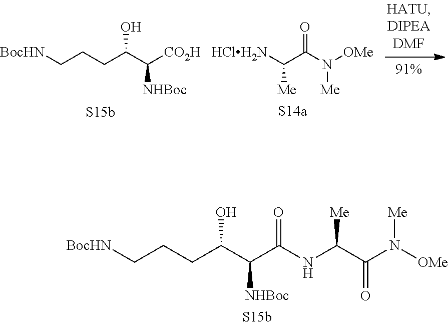

Compound S15b was prepared as described in a previously reported procedure using lysine hydroxylase KDO1.^{S1} Amine S14a was prepared as described previously.^{3x} Following General Procedure K: S15b (1.00 g, 2.76 mmol, 1 equiv), S14a (698 mg, 4.14 mmol, 1.5 equiv), HATU (1.10 g, 2.90 mmol, 1.05 equiv) and DIPEA (1.44 mL, 8.28 mmol, 3 equiv) were used to provide S15b (1.20 g, 91%) as a white solid.

¹H NMR (400 MHz, Methanol-d₄): δ 4.83 (d, J=7.2 Hz, 1H), 4.11-3.92 (m, 1H), 3.82 (s, 3H), 3.76-3.68 (m, 1H), 3.21 (s, 3H), 3.14-2.98 (m, 2H), 1.71-1.49 (m, 3H), 1.49-1.37 (m, 19H), 1.34 (d, J=7.1 Hz, 3H).

¹³C NMR (101 MHz, Methanol-d₄): δ 174.7, 173.1, 158.6, 157.8, 80.8, 79.8, 72.4, 62.1, 60.4, 47.5, 41.1, 32.5, 31.2, 28.9, 28.7, 27.1, 17.1.

HRMS (ESI): calculated for C₂₁H₄₁N₄O₈⁺ ([M+H]⁺) 477.2919, found 477.2916.

[α]_{D}²⁷=−28.1(c0.5,MeOH)

### di-tert-butyl ((4S,5S)-4-hydroxy-6-oxo-6-(((S)-1-oxopropan-2-yl)amino)hexane-1,5-diyl)dicarbamate (S16b)

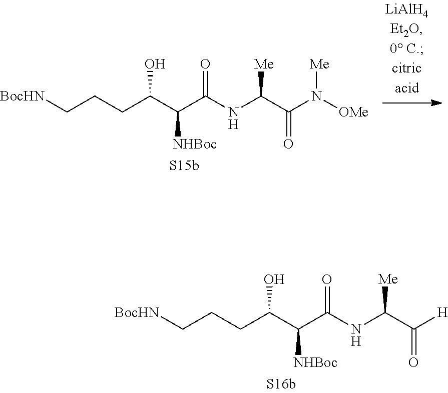 [SMILES: CON(C)C(=O)[C@H](C)NC(=O)[C@@H](NC(=O)OC(C)(C)C)[C@@H](O)CCCNC(=O)OC(C)(C)C ; C[C@@H](C=O)NC(=O)[C@@H](NC(=O)OC(C)(C)C)[C@@H](O)CCCNC(=O)OC(C)(C)C]

Following General Procedure B: S15b (500 mg, 1.05 mmol, 1 equiv) and LAH (149 mg, 3.94 mmol, 3.75 equiv) were used to provide S16b (332 mg) as a white solid used without further purification.

### tert-butyl (S,E)-4-((2S,3S)-2,6-bis((tert-butoxycarbonyl)amino)-3-hydroxyhexanamido)pent-2-enoate (S17b)

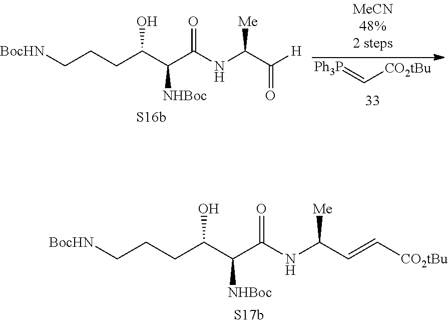

Following General Procedure C: S16b (332 mg) and 33 (374 mg, 0.994 mmol, 1.25 equiv) were used to provide S17b (262 mg, 48% over 2 steps) as a white solid.

¹H NMR (400 MHz, Methanol-d₄): δ 6.78 (dd, J=15.6, 4.8 Hz, 1H), 5.89 (dd, J=15.7, 1.8 Hz, 1H), 4.66-4.57 (m, 1H), 4.00 (d, J=6.9 Hz, 1H), 3.74 (ddd, J=9.7, 6.9, 2.6 Hz, 1H), 3.05 (td, J=6.6, 3.9 Hz, 2H), 1.74-1.49 (m, 4H), 1.47 (s, 9H), 1.46 (s, 9H), 1.43 (s, 9H), 1.29 (d, J=7.1 Hz, 3H).

¹³C NMR (101 MHz, Methanol-d₄): δ 172.7, 167.5, 158.6, 157.7, 149.2, 122.9, 81.7, 80.8, 79.9, 72.1, 60.9, 47.0, 41.2, 31.7, 28.8, 28.7, 28.4, 27.2, 19.9.

HRMS (ESI): calculated for C₂₅H₄₆N₃O₈⁺ ([M+H]⁺) 516.3279, found 516.3277.

[α]_{D}²⁷=−33.5(c1.1,MeOH)

### (S,E)-4-((2S,3S)-2,6-diamino-3-hydroxyhexanamido)pent-2-enoic Acid (S18b)

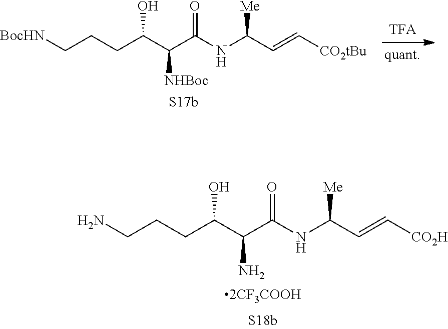 [SMILES: C[C@@H](/C=C/C(=O)OC(C)(C)C)NC(=O)[C@@H](NC(=O)OC(C)(C)C)[C@@H](O)CCCNC(=O)OC(C)(C)C ; C[C@@H](/C=C/C(=O)O)NC(=O)[C@@H](N)[C@@H](O)CCCN]

Following General Procedure D: S17b (100 mg, 0.194 mmol, 1 equiv) was used to provide S18b (100 mg, ˜quant.) as a white solid.

¹H NMR (400 MHz, Methanol-d₄): δ 6.87 (dd, J=15.7, 5.3 Hz, 1H), 5.88 (dd, J=15.7, 1.6 Hz, 1H), 4.67 (qdd, J=7.0, 5.1, 1.7 Hz, 1H), 4.07-3.99 (m, 1H), 3.93 (d, J=4.9 Hz, 1H), 2.97 (ddt, J=8.9, 6.7, 3.5 Hz, 2H), 2.00-1.87 (m, 1H), 1.82-1.69 (m, 1H), 1.55-1.48 (m, 2H), 1.33 (d, J=7.0 Hz, 3H).

¹³C NMR (101 MHz, Methanol-d₄): δ 169.6, 167.0, 149.8, 122.0, 70.0, 59.2, 47.4, 40.5, 29.6, 25.4, 19.6.

HRMS (ESI): calculated for C₁₁H₂₂N₃O₄⁺ ([M+H]⁺) 260.1605, found 260.1611.

[α]_{D}²⁴=−6.6(c1.2,MeOH)

### (5S,8S,9S,E)-8-amino-9-hydroxy-5-methyl-1,6-diazacyclododec-3-ene-2,7-dione (S19b)

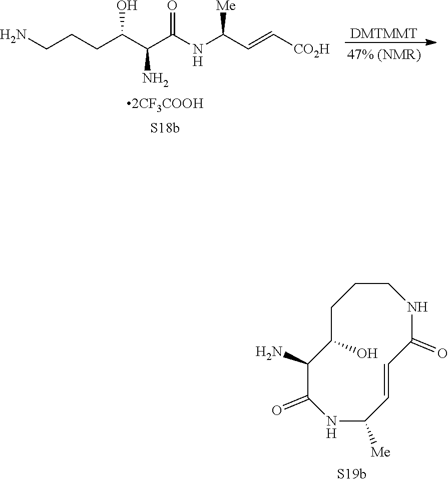

Following General Procedure E: S18b (80 mg, 0.164 mmol, 1 equiv) was used to provide S19b (57 mg, 47%) as a tan solid. Yield was determined by ¹H NMR analysis of a 6.2 mg sample of crude S18b and 6.5 mg of 4-toluenesulfonamide added as internal standard.

¹H NMR (400 MHz, DMSO-d₆): 4-toluenesulfonamide: δ 7.70 (d, J=7.8 Hz, 2H), 7.36 (d, J=7.8 Hz, 2H), 7.27 (br s, 2H), 2.37 (s, 3H). Compound S19b: δ 8.61 (d, J=7.4 Hz, 1H), 6.78 (d, J=14.2 Hz, 1H), 6.19 (d, J=15.1 Hz, 1H), 4.98-4.72 (m, 2H), 4.45-4.26 (m, 2H), 3.06-2.92 (m, 1H), 1.73 (d, J=11.4 Hz, 1H), 1.56-1.46 (m, 1H).

HRMS (ESI): calculated for C₁₁H₂₀N₃O₃⁺ ([M+H]⁺) 242.1499, found 242.1497.

### (2E,4E)-N-((2S,3R)-3-hydroxy-1-(((5S,8S,9S,E)-9-hydroxy-5-methyl-2,7-dioxo-1,6-diazacyclododec-3-en-8-yl)amino)-1-oxobutan-2-yl)-11-methyldodeca-2,4-dienamide (41)

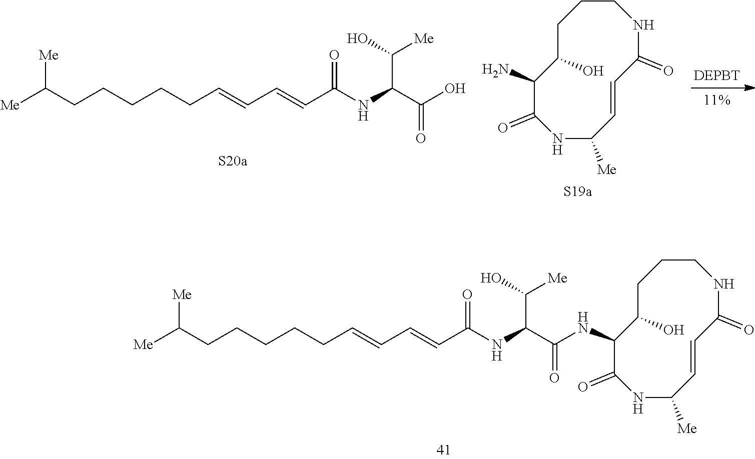 [SMILES: CC(C)CCCCC/C=C/C=C/C(=O)N[C@H](C(=O)O)[C@@H](C)O ; C[C@H]1/C=C/C(=O)NCCC[C@H](O)[C@H](N)C(=O)N1 ; CC(C)CCCCC/C=C/C=C/C(=O)N[C@H](C(=O)N[C@@H]1C(=O)N[C@@H](C)/C=C/C(=O)NCCC[C@@H]1O)[C@@H](C)O]

Following General Procedure F: Carboxylic acid S20a was synthesized as described previously.^{S6} S20a (39 mg, 0.125 mmol, 2 equiv), S19a (38 mg, 0.0626 mmol as judged by ¹H NMR, 1 equiv), DEPBT (37 mg, 0.125 mmol, 2 equiv) and DIPEA (65 μL, 0.375 mmol, 6 equiv) were used to provide 41 (3.6 mg, 11%) as a fluffy white solid.

¹H NMR (600 MHz, DMSO-d₆): δ 8.61 (d, J=7.5 Hz, 1H), 7.96 (d, J=8.4 Hz, 1H), 7.68 (d, J=7.9 Hz, 1H), 7.35 (dd, J=8.3, 6.0 Hz, 1H), 6.99 (dd, J=15.1, 10.8 Hz, 1H), 6.78 (dd, J=15.3, 4.7 Hz, 1H), 6.21-6.13 (m, 3H), 6.12-6.06 (m, 1H), 4.97 (d, J=4.8 Hz, 1H), 4.80 (d, J=6.5 Hz, 1H), 4.70 (dd, J=7.9, 3.9 Hz, 1H), 4.44 (dd, J=8.4, 4.3 Hz, 1H), 4.39-4.32 (m, 1H), 4.31-4.22 (m, 1H), 4.03-3.90 (m, 1H), 3.03-2.90 (m, 1H), 2.12 (q, J=7.2 Hz, 2H), 1.71 (td, J=12.4, 4.5 Hz, 1H), 1.55-1.45 (m, 1H), 1.38 (p, J=7.2 Hz, 2H), 1.34-1.28 (m, 2H), 1.27-1.23 (m, 4H), 1.19 (d, J=7.1 Hz, 3H), 1.16-1.12 (m, 3H), 1.04 (d, J=6.3 Hz, 3H), 0.84 (d, J=6.6 Hz, 6H).

¹³C NMR (151 MHz, DMSO-d₆): δ 170.7, 169.2, 165.6, 165.4, 146.9, 142.0, 139.7, 128.6, 123.1, 118.7, 67.5, 67.0, 58.0, 57.7, 45.9, 38.4, 37.8, 32.2, 28.9, 28.4, 27.4, 26.6, 26.4, 25.0, 22.5, 19.1, 18.2.

HRMS (ESI): calculated for C₂₈H₄₇N₄O₆⁺ ([M+H]⁺) 535.3490, found 535.3488.

[α]_{D}²⁷=−32.2(c0.1,MeOH)

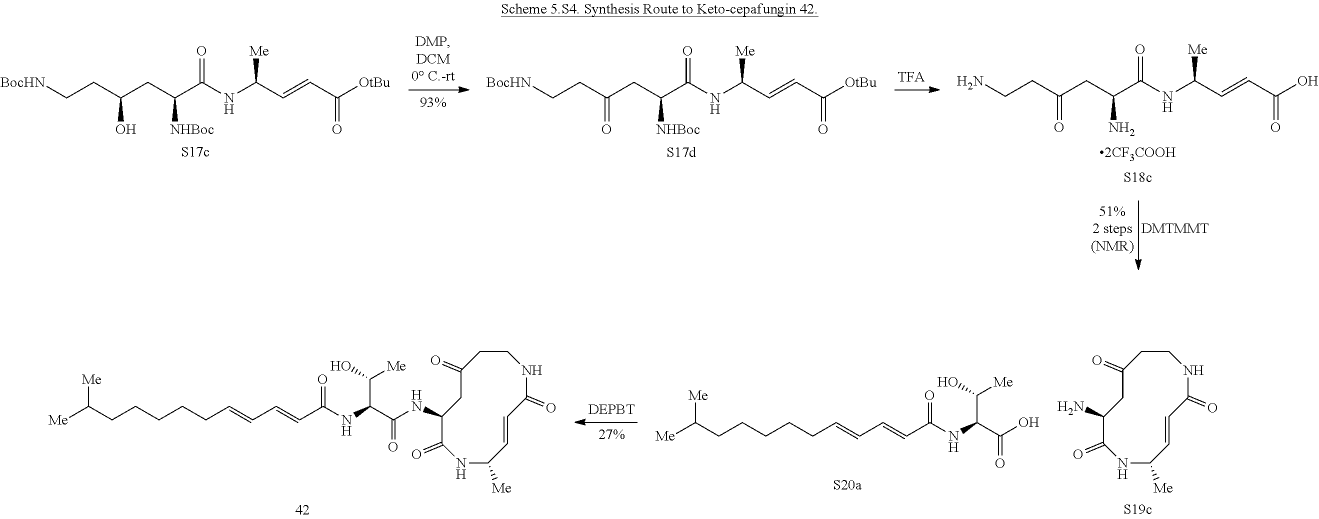

### tert-butyl (S,E)-4-((S)-2,6-bis((tert-butoxycarbonyl)amino)-4-oxohexanamido)pent-2-enoate (S17d)

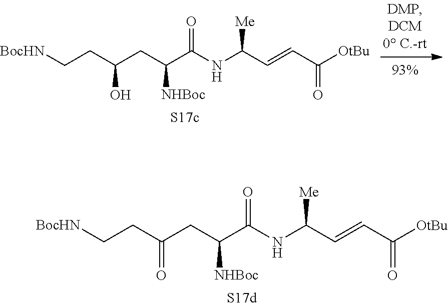 [SMILES: C[C@@H](/C=C/C(=O)OC(C)(C)C)NC(=O)[C@H](C[C@@H](O)CCNC(=O)OC(C)(C)C)NC(=O)OC(C)(C)C ; C[C@@H](/C=C/C(=O)OC(C)(C)C)NC(=O)[C@H](CC(=O)CCNC(=O)OC(C)(C)C)NC(=O)OC(C)(C)C]

Compound S17c was synthesized as previously reported.^{S6} A 50 mL round-bottom flask was charged with S17c (171 mg, 0.332 mmol, 1 equiv), dissolved in 17 mL anhydrous DCM, and cooled to 0° C. Dess-Martin Periodinane (169 mg, 0.398 mmol, 1.2 equiv) was added and the reaction was stirred under argon to rt overnight. The mixture was diluted with 6 mL Et₂O, filtered through a plug of celite and concentrated. The residue was purified by flash chromatography (70:29:1 DCM:EtOAc:MeOH) to provide S17d (159 mg, 93%) as a white solid.

¹H NMR (400 MHz, Methanol-d₄): δ 6.77 (dd, J=15.7, 4.8 Hz, 1H), 5.83 (dd, J=15.7, 1.8 Hz, 1H), 4.62-4.52 (m, 1H), 4.42 (d, J=6.6 Hz, 1H), 3.28 (t, J=7.1 Hz, 2H), 2.87 (tt, J=17.4, 9.5 Hz, 2H), 2.65 (t, J=6.5 Hz, 2H), 1.47 (s, 9H), 1.46 (s, 9H), 1.42 (s, 9H), 1.28 (d, J=7.1 Hz, 3H).

¹³C NMR (101 MHz, Methanol-d₄): δ 208.0, 173.5, 167.4, 158.3, 157.7, 149.1, 122.9, 81.7, 80.9, 80.1, 52.0, 47.1, 45.0, 43.7, 36.4, 28.8, 28.7, 28.4, 19.8.

HRMS (ESI): calculated for C₂₅H₄₄N₃O₈⁺ ([M+H]⁺) 514.3123, found 514.3122.

[α]_{D}²⁷=−17.3(c1.5,MeOH)

### (S,E)-4-((S)-2,6-diamino-4-oxohexanamido)pent-2-enoic Acid (S18c)

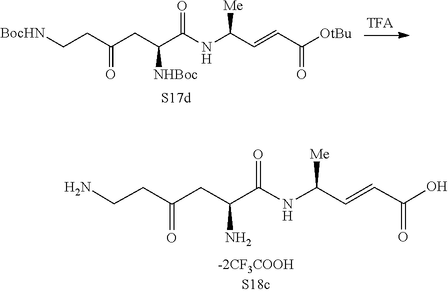

Following General Procedure D: S17d (76 mg, 0.148 mmol, 1 equiv) was used to provide S18c (71 mg) as a white solid used without further purification.

¹H NMR (600 MHz, Methanol-d₄): δ 6.81 (dd, J=15.7, 5.1 Hz, 1H), 5.84 (dd, J=15.7, 1.7 Hz, 1H), 4.62 (ddt, J=10.5, 7.1, 3.6 Hz, 1H), 4.25-4.19 (m, 1H), 3.26-3.10 (m, 4H), 2.98 (q, J=6.1 Hz, 2H), 1.57 (dd, J=11.8, 7.0 Hz, 1H), 1.32 (d, J=7.1 Hz, 3H).

HRMS (ESI): calculated for C₁₁H₂₀N₃O₄⁺ ([M+H]⁺) 258.1448, found 258.1447.

### (5S,8S,E)-8-amino-5-methyl-1,6-diazacyclododec-3-ene-2,7,10-trione (S19c)

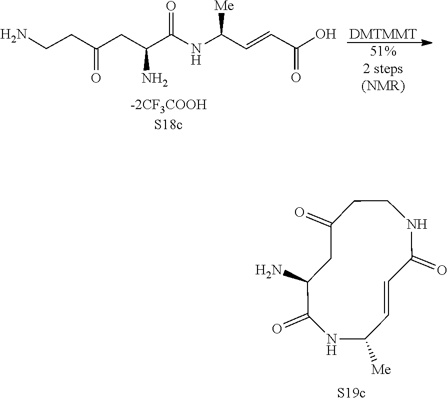 [SMILES: C[C@@H](/C=C/C(=O)O)NC(=O)[C@@H](N)CC(=O)CCN ; C[C@H]1/C=C/C(=O)NCCC(=O)C[C@H](N)C(=O)N1]

Following General Procedure E: S18c (58 mg, 0.119 mmol, 1 equiv) was used to provide S19c (72 mg, 51% over 2 steps) as a tan solid. Yield was determined by ¹H NMR analysis of a 2.4 mg sample of crude S19c and 2.8 mg of 4-toluenesulfonamide added as internal standard.

¹H NMR (400 MHz, Methanol-d₄): 4-toluenesulfonamide: δ 7.86-7.63 (m, 2H), 7.41-7.26 (m, 2H), 2.42 (s, 3H). Compound S19c: δ 6.86 (dd, J=15.5, 5.1 Hz, 1H), 6.00-5.92 (m, 1H), 4.67-4.56 (m, 1H), 3.16-3.11 (m, 1H), 3.03-2.92 (m, 1H), 1.54 (s, 1H), 1.31 (d, J=7.2 Hz, 3H).

HRMS (ESI): calculated for C₁₁H₁₈N₃O₃⁺ ([M+H]⁺) 240.1343, found 240.1333.

### (2E,4E)-N-((2S,3R)-3-hydroxy-1-(((5S,8S,E)-5-methyl-2,7,10-trioxo-1,6-diazacyclododec-3-en-8-yl)amino)-1-oxobutan-2-yl)-11-methyldodeca-2,4-dienamide (42)

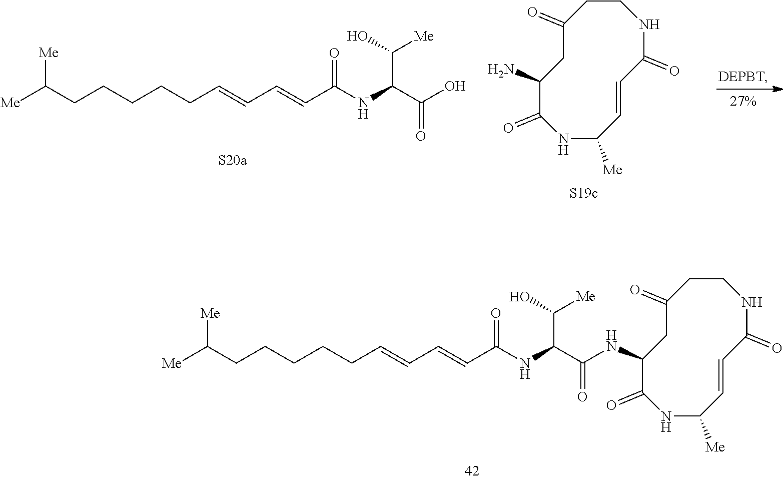 [SMILES: CC(C)CCCCC/C=C/C=C/C(=O)N[C@H](C(=O)O)[C@@H](C)O ; C[C@H]1/C=C/C(=O)NCCC(=O)C[C@H](N)C(=O)N1 ; CC(C)CCCCC/C=C/C=C/C(=O)N[C@H](C(=O)N[C@H]1CC(=O)CCNC(=O)/C=C/[C@H](C)NC1=O)[C@@H](C)O]

Following General Procedure F: Compound S20a was synthesized by a previously reported procedure.^{S6} S20a (38 mg, 0.122 mmol, 2 equiv), S19c (69 mg, 0.0612 mmol as judged by ¹H NMR analysis, 1 equiv), DEPBT (37 mg, 0.122 mmol, 2 equiv) and DIPEA (64 μL, 0.367 mmol, 6 equiv) were used to provide 42 (4.2 mg, 27%) as a fluffy white solid.

¹H NMR (600 MHz, DMSO-d₆): δ 8.60 (d, J=7.1 Hz, 1H), 7.94 (d, J=8.6 Hz, 1H), 7.80 (d, J=7.9 Hz, 1H), 7.51 (dd, J=7.1, 5.4 Hz, 1H), 7.00 (dd, J=15.1, 10.8 Hz, 1H), 6.61 (dd, J=15.4, 4.9 Hz, 1H), 6.22-6.06 (m, 3H), 5.72 (dd, J=15.4, 1.4 Hz, 1H), 4.93 (d, J=5.1 Hz, 1H), 4.66 (ddd, J=10.2, 7.9, 4.0 Hz, 1H), 4.37-4.26 (m, 2H), 4.00-3.91 (m, 1H), 3.62 (dq, J=14.9, 4.7 Hz, 1H), 3.04-2.95 (m, 1H), 2.95-2.82 (m, 2H), 2.67 (dd, J=13.9, 4.0 Hz, 1H), 2.39 (dt, J=18.4, 4.4 Hz, 1H), 2.13 (q, J=7.2 Hz, 2H), 1.53-1.46 (m, 1H), 1.38 (p, J=7.2 Hz, 2H), 1.29-1.22 (m, 4H), 1.17-1.11 (m, 5H), 1.00 (d, J=6.3 Hz, 3H), 0.84 (d, J=6.6 Hz, 6H).

¹³C NMR (151 MHz, DMSO-d₆): δ 207.3, 169.7, 169.3, 166.3, 165.5, 146.0, 142.1, 139.8, 128.6, 123.0, 119.5, 66.7, 58.2, 50.4, 46.8, 45.7, 45.6, 38.4, 34.3, 32.2, 28.8, 28.4, 27.4, 26.6, 22.5, 19.8, 18.2.

HRMS (ESI): calculated for C₂₈H₄₅N₄O₆⁺ ([M+H]⁺) 533.3334, found 533.3321.

[α]_{D}²⁷=−81.5(c0.1,MeOH)

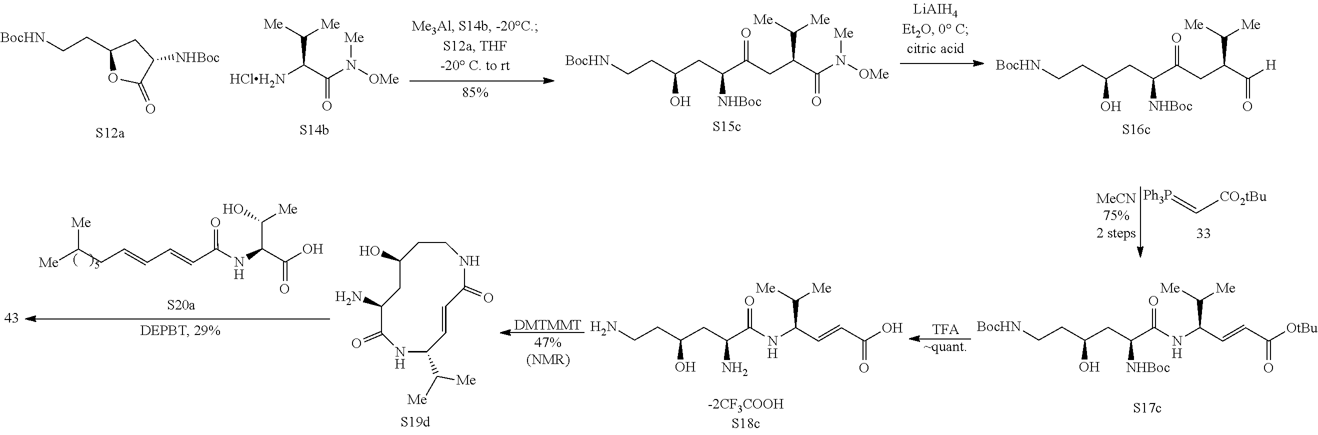

### (S)-2-amino-N-methoxy-N,3-dimethylbutanamide (S14b)

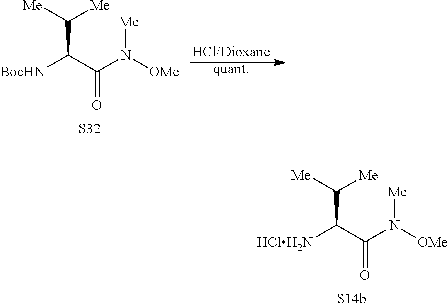

Following a previously reported procedure,^{S6} a flame-dried 100 mL round-bottom flask was charged with S32 (3.85 g, 14.8 mmol, 1 equiv) and dissolved in 24 mL 4 M HCl/dioxane solution. The mixture was stirred under argon at rt for 1 h, then concentrated in vacuo to dryness. The residue was then evaporated from 50 mL 1:1 MeOH:toluene, then twice again from 50 mL toluene to provide S14b (3.19 g, quant.) as a clear, colorless solid used without further purification.

¹H NMR (400 MHz, Methanol-d₄): δ 4.22 (d, J=5.3 Hz, 1H), 3.80 (s, 3H), 3.27 (s, 3H), 2.28 (dhept, J=7.1, 5.4 Hz, 1H), 1.05 (dd, J=22.6, 7.0 Hz, 6H).

¹³C NMR (101 MHz, Methanol-d₄): δ 169.8, 62.3, 56.9, 32.3, 30.8, 19.2, 17.4.

HRMS (ESI): calculated for C₇H₁₇N₂O₂⁺ ([M+H]⁺) 161.1285, found 161.1277.

[α]_{D}²⁷=−0.9(c1.2,MeOH)

### di-tert-butyl ((3S,5S)-3-hydroxy-6-(((S)-1-(methoxy(methyl)amino)-3-methyl-1-oxobutan-2-yl)amino)-6-oxohexane-1,5-diyl)dicarbamate (S15c)

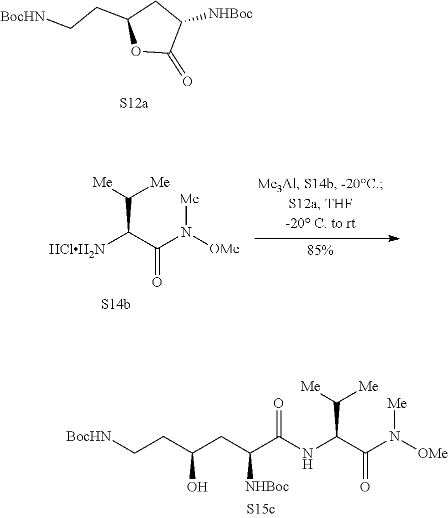

Compound S12a was synthesized as described previously.^{S6} Following General Procedure A: S12a (1.02 g, 2.96 mmol, 1 equiv) and S14b (3.19 g, 14.8 mmol, 5 equiv) were used to provide S15c (1.27 g, 85%) as a white solid.

¹H NMR (400 MHz, Methanol-d₄): δ 4.81 (d, J=6.5 Hz, 1H), 4.20 (t, J=7.4 Hz, 1H), 3.81 (s, 3H), 3.72-3.63 (m, 1H), 3.21 (s, 3H), 3.18-3.11 (m, 2H), 2.07 (hept, J=6.8 Hz, 1H), 1.93-1.78 (m, 1H), 1.72-1.60 (m, 2H), 1.57-1.50 (m, 1H), 1.48-1.39 (m, 18H), 0.95 (t, J=6.6 Hz, 6H).

¹³C NMR (101 MHz, Methanol-d₄): δ 174.9, 173.6, 158.6, 157.7, 80.7, 80.0, 67.3, 62.1, 55.4, 53.7, 40.6, 38.4, 38.1, 32.2, 32.1, 28.8, 28.7, 19.6, 18.4.

HRMS (ESI): calculated for C₂₃H₄₅N₄O₈⁺ ([M+H]⁺) 505.3232, found 505.3231.

[α]_{D}²⁷=−37.7(c0.6,MeOH)

### di-tert-butyl ((3S,5S)-3-hydroxy-6-(((S)-3-methyl-1-oxobutan-2-yl)amino)-6-oxohexane-1,5-diyl)dicarbamate (S16c)

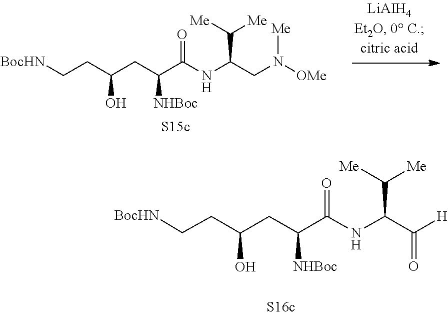

Following General Procedure B: S15c (600 mg, 1.19 mmol, 1 equiv) and LAH (169 mg, 4.46 mmol, 3.75 equiv) were used to provide S16c (519 mg) as a white solid that was directly subjected to the next reaction.

¹H NMR (400 MHz, CDCl₃): δ 9.61 (d, J=4.0 Hz, 1H), 7.25-7.09 (m, 1H), 5.87-5.57 (m, 1H), 4.92-4.78 (m, 1H), 4.55-4.39 (m, 1H), 4.38-4.12 (m, 2H), 3.84-3.72 (m, 1H), 3.51-3.40 (m, 1H), 3.17-3.05 (m, 1H), 2.39-2.25 (m, 1H), 2.03-1.93 (m, 1H), 1.84-1.77 (m, 1H), 1.62-1.55 (m, 2H), 1.46-1.41 (m, 18H), 1.02-0.93 (m, 6H).

### tert-butyl (S,E)-4-((2S,4S)-2,6-bis((tert-butoxycarbonyl)amino)-4-hydroxyhexanamido)-5-methylhex-2-enoate (S17c)

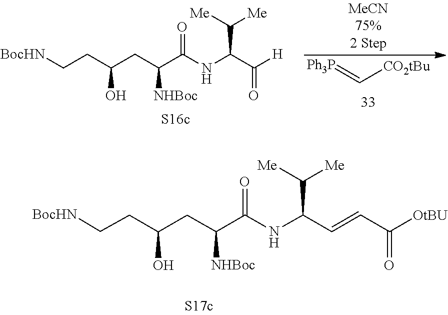

Following General Procedure C: S16c (519 mg) was used to provide S17c (487 mg, 75% over 2 steps) as a white solid.

¹H NMR (400 MHz, Methanol-d₄): δ 6.79 (dd, J=15.7, 5.9 Hz, 1H), 5.86 (dd, J=15.7, 1.6 Hz, 1H), 4.34 (t, J=6.3 Hz, 1H), 4.18 (t, J=7.4 Hz, 1H), 3.70-3.61 (m, 1H), 3.16 (dd, J=7.7, 6.0 Hz, 2H), 1.96-1.82 (m, 2H), 1.74-1.63 (m, 2H), 1.61-1.51 (m, 1H), 1.50-1.40 (m, 27H), 0.97-0.90 (m, 6H).

¹³C NMR (101 MHz, Methanol-d₄): δ 174.7, 167.2, 158.7, 157.7, 147.1, 124.4, 81.8, 80.7, 80.0, 67.6, 56.9, 54.2, 40.5, 38.4, 38.1, 33.4, 28.8, 28.8, 28.4, 19.6, 18.7.

HRMS (ESI): calculated for C₂₇H₅₀N₃O₈⁺ ([M+H]⁺) 544.3592, found 544.3591.

[α]_{D}²⁷=−35.3(c0.5,MeOH)

### (S,E)-4-((2S,4S)-2,6-diamino-4-hydroxyhexanamido)-5-methylhex-2-enoic Acid (S18c)

Following General Procedure D: S17c (150 mg, 0.276 mmol, 1 equiv) was used to provide

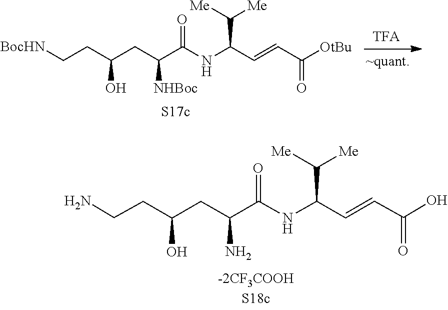

S18c (143 mg, quant.) as a white solid.

¹H NMR (400 MHz, Methanol-d₄): δ 6.81 (dd, J=15.7, 6.6 Hz, 1H), 5.90 (dd, J=15.7, 1.4 Hz, 1H), 4.44-4.30 (m, 1H), 4.08 (dd, J=8.1, 5.2 Hz, 1H), 4.00-3.86 (m, 1H), 3.15-2.99 (m, 2H), 2.06-1.97 (m, 1H), 1.97-1.72 (m, 4H), 1.03-0.92 (m, 6H).

¹³C NMR (101 MHz, Methanol-d₄): δ 169.9, 169.8, 146.9, 124.3, 68.4, 57.7, 53.6, 39.2, 37.8, 36.0, 33.2, 19.3, 19.0.

HRMS (ESI): calculated for C₁₃H₂₆N₃O₄+([M+H]⁺) 288.1918, found 288.1917.

[α]_{D}²⁷=+2.6(c0.4,MeOH)

### (5S,8S,10S,E)-8-amino-10-hydroxy-5-isopropyl-1,6-diazacyclododec-3-ene-2,7-dione (S19d)

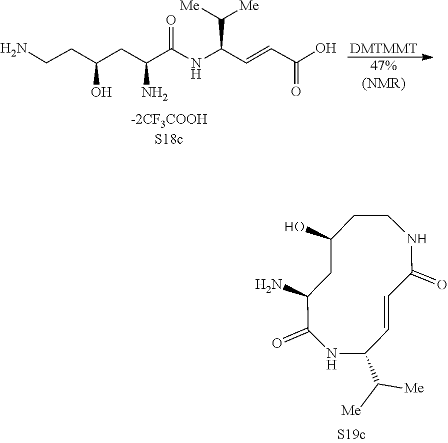 [SMILES: CC(C)[C@@H](/C=C/C(=O)O)NC(=O)[C@@H](N)C[C@@H](O)CCN ; CC(C)[C@H]1/C=C/C(=O)NCC[C@H](O)C[C@H](N)C(=O)N1]

Following General Procedure E: S18c (131 mg, 0.254 mmol, 1 equiv) was used to provide S19d (138 mg, 47%). Yield was determined by ¹H NMR analysis of a 8.6 mg sample of crude S19d and 7.1 mg of 4-toluenesulfonamide added as internal standard.

¹H NMR (400 MHz, Methanol-d₄): 4-toluenesulfonamide: δ 7.78 (d, J=8.2 Hz, 2H), 7.35 (d, J=8.0 Hz, 2H), 2.41 (s, 3H). Compound S19d: δ 6.59 (dd, J=16.1, 6.9 Hz, 1H), 6.40 (dd, J=16.1, 10.6 Hz, 1H), 4.36-4.24 (m, 1H), 3.98-3.88 (m, 4H), 2.26-2.14 (m, 1H), 1.97-1.75 (m, 3H), 1.68 (s, 2H), 1.03 (dd, J=13.4, 6.7 Hz, 6H).

HRMS (ESI): calculated for C₁₃H₂₄N₃O₃⁺ ([M+H]⁺) 270.1812, found 270.1815.

### (2E,4E)-N-((2S,3R)-3-hydroxy-1-(((5S,8S,10S,E)-10-hydroxy-5-isopropyl-2,7-dioxo-1,6-diazacyclododec-3-en-8-yl)amino)-1-oxobutan-2-yl)-11-methyldodeca-2,4-dienamide (43)

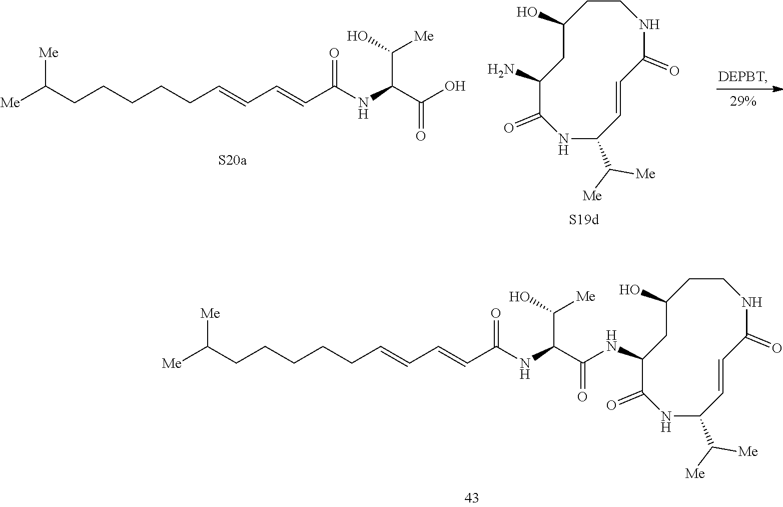 [SMILES: CC(C)CCCCC/C=C/C=C/C(=O)N[C@H](C(=O)O)[C@@H](C)O ; CC(C)[C@H]1/C=C/C(=O)NCC[C@H](O)C[C@H](N)C(=O)N1 ; CC(C)CCCCC/C=C/C=C/C(=O)N[C@H](C(=O)N[C@H]1C[C@@H](O)CCNC(=O)/C=C/[C@H](C(C)C)NC1=O)[C@@H](C)O]

Compound S20a was synthesized by a previously reported procedure. Following General Procedure F: S19d (60 mg, 0.0573 mmol as judged by ¹H NMR analysis, 1 equiv) and S20a (36 mg, 0.115 mmol, 2 equiv) were used to provide 43 (9.2 mg, 29%) as a fluffy white powder.

¹H NMR (600 MHz, DMSO-d₆): δ 8.48 (d, J=8.6 Hz, 1H), 7.93 (d, J=8.7 Hz, 1H), 7.78 (d, J=7.6 Hz, 1H), 7.41 (t, J=6.3 Hz, 1H), 6.99 (dd, J=15.1, 10.8 Hz, 1H), 6.32 (dd, J=15.8, 6.7 Hz, 1H), 6.24-6.01 (m, 4H), 4.85 (br s, 1H), 4.67 (br s, 1H), 4.37 (t, J=9.9 Hz, 1H), 4.28 (dd, J=8.8, 4.5 Hz, 1H), 4.05 (q, J=8.0 Hz, 1H), 3.98-3.82 (m, 1H), 3.62-3.52 (m, 1H), 3.05-2.92 (m, 2H), 2.13 (q, J=7.2 Hz, 2H), 1.86 (td, J=12.5, 6.8 Hz, 1H), 1.81-1.69 (m, 1H), 1.57 (d, J=13.3 Hz, 1H), 1.50 (hept, J=6.6 Hz, 1H), 1.46-1.34 (m, 4H), 1.29-1.22 (m, 4H), 1.17-1.11 (m, 2H), 0.99 (d, J=6.3 Hz, 3H), 0.91 (d, J=6.6 Hz, 3H), 0.88 (d, J=6.7 Hz, 3H), 0.84 (d, J=6.6 Hz, 6H).

¹³C NMR (151 MHz, DMSO-d₆): δ 171.5, 169.4, 167.8, 165.4, 142.0, 140.8, 139.7, 128.6, 124.8, 123.1, 66.9, 66.8, 58.2, 55.6, 51.2, 42.2, 38.4, 32.2, 30.6, 28.8, 28.4, 27.4, 26.6, 22.5, 19.8, 19.6, 19.1.

HRMS (ESI): calculated for C₃₀H₅₁N₄O₆⁺ ([M+H]⁺) 563.3803, found 563.3804.

[α]_{D}²⁷=−135.3(c0.2,MeOH)

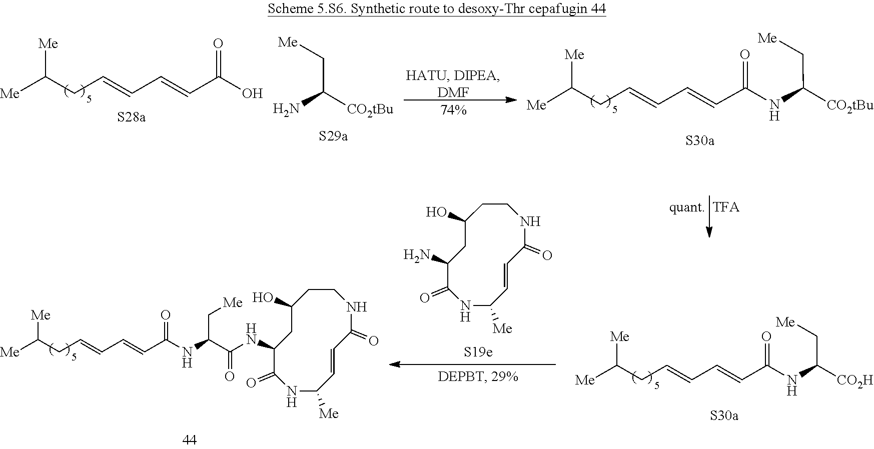

### tert-butyl (S)-2-((2E,4E)-11-methyldodeca-2,4-dienamido)butanoate (S30a)

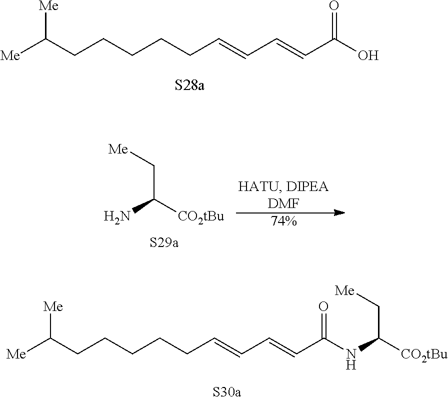 [SMILES: CC(C)CCCCC/C=C/C=C/C(=O)O ; CC[C@H](N)C(=O)OC(C)(C)C ; CC[C@H](NC(=O)/C=C/C=C/CCCCCC(C)C)C(=O)OC(C)(C)C]

Compound S28a was synthesized as described previously. ^{S6} Following General Procedure K: S28a (500 mg, 2.38 mmol, 1 equiv), S29a (698 mg, 3.57 mmol, 1.5 equiv), HATU (949 mg, 2.50 mmol, 1.05 equiv) and DIPEA (1.24 mL, 7.13 mmol, 3 equiv) were used to provide S30a as a colorless resin.

¹H NMR (400 MHz, CDCl₃): δ 7.18 (dd, J=15.0, 10.1 Hz, 1H), 6.21-5.99 (m, 3H), 5.79 (d, J=15.1 Hz, 1H), 4.57 (dt, J=8.0, 5.8 Hz, 1H), 2.14 (q, J=7.0 Hz, 2H), 1.90 (dqd, J=15.1, 7.5, 5.4 Hz, 1H), 1.79-1.69 (m, 1H), 1.55-1.48 (m, 1H), 1.47 (s, 9H), 1.40 (q, J=6.8 Hz, 2H), 1.30-1.23 (m, 4H), 1.18-1.11 (m, 2H), 0.89 (t, J=7.4 Hz, 3H), 0.85 (d, J=6.6 Hz, 6H).

¹³C NMR (101 MHz, CDCl₃): δ 172.0, 165.9, 143.7, 141.9, 128.3, 121.5, 82.2, 53.7, 39.0, 33.1, 29.5, 28.9, 28.2, 28.1, 27.3, 26.0, 22.8, 9.3.

HRMS (ESI): calculated for C₂₁H₃₈NO₃⁺ ([M+H]⁺) 352.2846, found 352.2845.

[α]_{D}²⁷=−40.6(c0.6,MeOH)

### (S)-2-((2E,4E)-11-methyldodeca-2,4-dienamido)butanoic Acid (S31a)

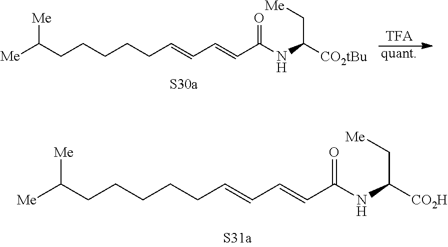 [SMILES: CC[C@H](NC(=O)/C=C/C=C/CCCCCC(C)C)C(=O)OC(C)(C)C ; CC[C@H](NC(=O)/C=C/C=C/CCCCCC(C)C)C(=O)O]

Following General Procedure L: S30a (60 mg, 0.171 mmol, 1 equiv) was used to provide S31a (57 mg, quant.) as a clear, colorless resin.

¹H NMR (400 MHz, CDCl₃): δ 7.25-7.18 (m, 1H), 6.37 (br s, 1H), 6.21 (d, J=7.5 Hz, 1H), 6.17-6.06 (m, 2H), 5.82 (d, J=15.0 Hz, 1H), 4.63 (td, J=7.1, 5.5 Hz, 1H), 2.15 (q, J=7.0 Hz, 2H), 2.06-1.94 (m, 1H), 1.88-1.75 (m, 1H), 1.57-1.45 (m, 1H), 1.41 (p, J=7.0 Hz, 2H), 1.30-1.24 (m, 4H), 1.18-1.11 (m, 2H), 0.97 (t, J=7.5 Hz, 3H), 0.86 (d, J=6.6 Hz, 6H).

¹³C NMR (101 MHz, CDCl₃): δ 175.3, 167.3, 145.0, 143.3, 128.2, 120.4, 53.9, 39.0, 33.2, 29.6, 28.9, 28.1, 27.3, 25.3, 22.8, 9.7.

HRMS (ESI): calculated for C₁₇H₃₀NO₃⁺ ([M+H]⁺) 296.2220, found 296.2225.

[α]_{D}²⁶=−15.7(c0.4,MeOH)

### (2E,4E)-N—((S)-1-(((5S,8S,10S,E)-10-hydroxy-5-methyl-2,7-dioxo-1,6-diazacyclododec-3-en-8-yl)amino)-1-oxobutan-2-yl)-11-methyldodeca-2,4-dienamide (44)

Compound S19e was synthesized as previously described.^{S6} Following General Procedure F:

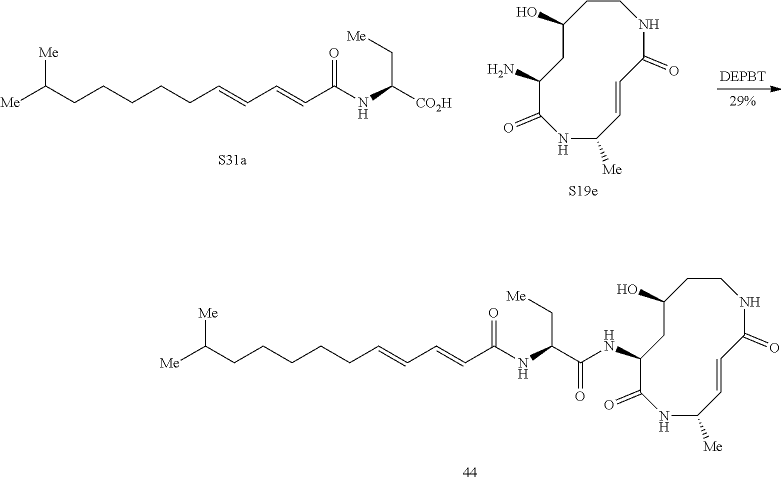 [SMILES: CC[C@H](NC(=O)/C=C/C=C/CCCCCC(C)C)C(=O)O ; C[C@H]1/C=C/C(=O)NCC[C@H](O)C[C@H](N)C(=O)N1 ; CC[C@H](NC(=O)/C=C/C=C/CCCCCC(C)C)C(=O)N[C@H]1C[C@@H](O)CCNC(=O)/C=C/[C@H](C)NC1=O]

S31a (50 mg, 0.169 mmol, 2 equiv) and S19e (102 mg, 0.847 mmol as judged by ¹H NMR analysis, 1 equiv) were used to provide 44 (12.8 mg, 29%) as a fluffy white powder.

¹H NMR (600 MHz, DMSO-d₆): δ 8.80-8.40 (m, 1H), 8.03 (d, J=8.2 Hz, 1H), 7.96 (d, J=7.7 Hz, 1H), 7.41 (t, J=6.2 Hz, 1H), 6.98 (dd, J=15.1, 10.8 Hz, 1H), 6.40 (d, J=14.1 Hz, 1H), 6.18 (dt, J=15.2, 5.0 Hz, 2H), 6.11-6.00 (m, 2H), 4.82-4.51 (m, 1H), 4.38 (s, 1H), 4.34-4.20 (m, 2H), 3.56 (s, 1H), 3.07-2.94 (m, 2H), 2.12 (q, J=7.2 Hz, 2H), 1.97-1.77 (m, 1H), 1.67-1.57 (m, 1H), 1.57-1.42 (m, 4H), 1.41-1.34 (m, 3H), 1.31-1.22 (m, 5H), 1.22-1.18 (m, 2H), 1.14 (q, J=6.9 Hz, 2H), 0.84 (d, J=6.6 Hz, 6H), 0.81 (t, J=7.4 Hz, 3H).

¹³C NMR (151 MHz, DMSO-d₆): δ 171.1, 170.8, 167.6, 165.1, 143.2, 142.0, 139.6, 128.6, 123.2, 123.0, 67.0, 53.6, 51.2, 44.8, 42.4, 38.4, 32.2, 28.8, 28.4, 27.4, 26.6, 25.5, 22.5, 18.6, 10.2.

HRMS (ESI): calculated for C₂₈H₄₇N₄O₅⁺ ([M+H]⁺) 519.3541, found 519.3544.

[α]_{D}²⁷=−125.7(c0.2,MeOH)

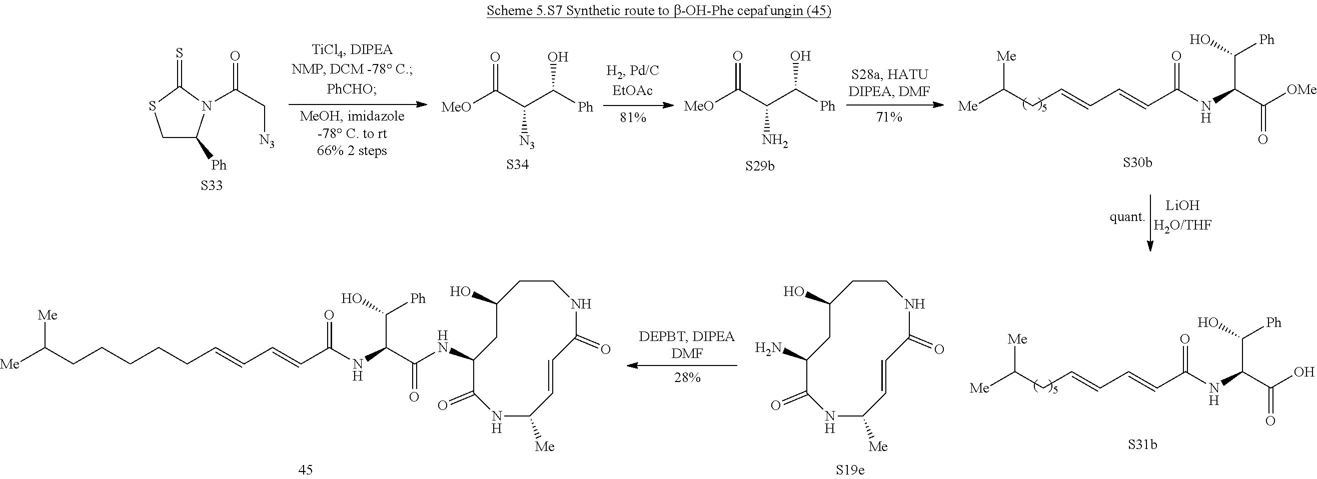

### methyl (2S,3R)-2-azido-3-hydroxy-3-phenylpropanoate (S34)

Compound S33 was prepared from (S)-2-amino-2-phenylethan-1-ol following previously reported procedures.^{S8} Following a procedure adapted from Patel et al.,^{S9} in a flame-dried 100 mL round bottom flask S33 (1.00 g, 3.59 mmol, 1 equiv) was dissolved in 36 mL anhydrous DCM and cooled to −78° C. Neat TiCl₄ (414 μL, 3.77 mmol, 1.05 equiv) was added dropwise, followed by DIPEA (688 μL, 3.95 mmol, 1.1 equiv). The mixture was stirred for 1 h under argon at −78° C. Then, N-methylpyrrolidone (693 μL, 7.19 mmol, 2 equiv) was added and stirred a further 15 min. A solution of benzaldehyde (548 μL, 5.39 mmol, 1.5 equiv) in 2 mL anhydrous DCM was added dropwise, and the reaction was stirred under argon at −78° C. for 6 h or until complete consumption of S33 as judged by TLC. At −78° C., a solution of imidazole (4.89 g, 71.9 mmol, 20 equiv) dissolved in 31 mL anhydrous MeOH was slowly added and the mixture was warmed to −35° C. over 1.5 h. The reaction was then quenched with 50 mL H₂O and the organics were removed in vacuo. The aqueous phase was diluted with 100 mL H₂O and extracted with 5×30 mL DCM. The combined organic layers were washed with 30 mL 1 M HCl, H₂O, NaHCO₃, and brine, dried over Na₂SO₄, filtered and concentrated. The residue was precipitated into 45 mL cyclohexane from 5 mL DCM, and the solids were collected by centrifugation (4° C., 10 min, 4200 rpm). The supernatant was concentrated and purified by flash chromatography (100% DCM) to provide S34 (527 mg, 66% over 2 steps) as an oil.

¹H NMR (400 MHz, CDCl₃): δ 7.42-7.31 (m, 5H), 5.21 (d, J=4.3 Hz, 1H), 4.05 (d, J=4.3 Hz, 1H), 3.77 (s, 3H), 2.67 (s, 1H).

¹³C NMR (101 MHz, CDCl₃): δ 169.17, 139.27, 128.75, 128.66, 126.23, 74.56, 67.78, 52.98. HRMS (ESI): calculated for C₁₀H₁₁N₃NaO₃⁺ ([M+Na]+) 244.0693, found 244.0692.

[α]_{D}²⁷=−77.2(c2.4,MeOH)

### methyl (2S,3R)-2-amino-3-hydroxy-3-phenylpropanoate (S29b)

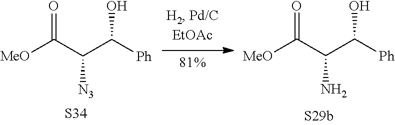 [SMILES: COC(=O)[C@@H](N=[N+]=[N-])[C@H](O)c1ccccc1 ; COC(=O)[C@@H](N)[C@H](O)c1ccccc1 ; "S34"]

In a 25 mL reaction vial, azide S34 (200 mg, 0.904 mmol, 1 equiv) was dissolved in 6 mL EtOAc, then 10% Pd/C (40 mg, 20 wt % relative to S34) was added. The mixture was sparged with argon for 25 min, then with H₂ for 15 min, then stirred under an atmosphere of H₂ overnight. An additional 10 wt % Pd/C were added twice over the course of 2 days. At 72 h, the reaction was filtered through a plug of celite then purified by flash chromatography (95:5 DCM:MeOH) to provide S29b (118 mg) as a colorless oil.

¹H NMR (400 MHz, CDCl₃): δ 7.38-7.27 (m, 5H), 4.97 (d, J=5.4 Hz, 1H), 3.86-3.81 (m, 1H), 3.61 (s, 3H), 3.01 (br s, 2H), 1.25 (d, J=1.9 Hz, 1H).

¹³C NMR (101 MHz, CDCl₃): δ 172.4, 140.4, 128.6, 128.2, 126.3, 73.8, 60.5, 52.6.

HRMS (ESI): calculated for C₁₀H₁₄NO₃⁺ ([M+H]⁺) 196.0968, found 196.0969.

[α]_{D}²⁷=+12.8(c0.3,MeOH)

### methyl (2S,3R)-3-hydroxy-2-((2E,4E)-11-methyldodeca-2,4-dienamido)-3-phenylpropanoate (S30b)

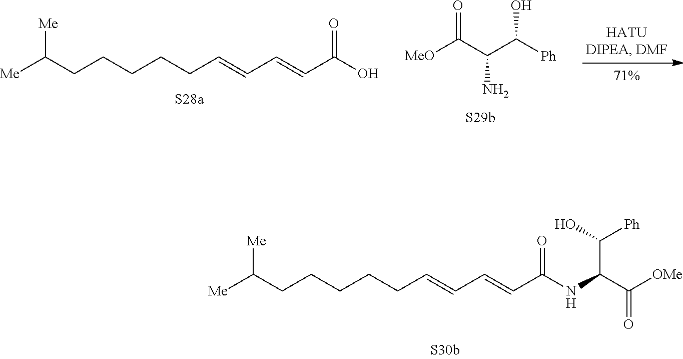 [SMILES: CC(C)CCCCC/C=C/C=C/C(=O)O ; COC(=O)[C@@H](N)[C@H](O)c1ccccc1 ; COC(=O)[C@@H](NC(=O)/C=C/C=C/CCCCCC(C)C)[C@H](O)c1ccccc1]

Following General Procedure K: S28a (95 mg, 0.451 mmol, 1 equiv), S29b (80 mg, 0.496 mmol, 1.1 equiv), HATU (180 mg, 0.474 mmol, 1.05 equiv) and DIPEA (94 μL, 0.541 mmol, 1.2 equiv) were used to provide S30b (124 mg, 71%) as a clear, colorless resin.

¹H NMR (400 MHz, CDCl₃): δ 7.38-7.26 (m, 5H), 7.12 (dd, J=15.0, 9.8 Hz, 1H), 6.24 (d, J=8.6 Hz, 1H), 6.15-5.99 (m, 2H), 5.75 (d, J=15.0 Hz, 1H), 5.28 (d, J=3.4 Hz, 1H), 4.95 (dd, J=8.5, 3.5 Hz, 1H), 3.72 (s, 3H), 2.13 (q, J=6.7 Hz, 2H), 1.57-1.45 (m, 1H), 1.46-1.34 (m, 2H), 1.30-1.22 (m, 4H), 1.19-1.09 (m, 2H), 0.86 (d, J=6.7 Hz, 6H).

¹³C NMR (101 MHz, CDCl₃): δ 171.2, 166.7, 144.4, 142.8, 139.7, 128.6, 128.3, 128.2, 126.0, 120.6, 74.2, 58.4, 52.8, 39.0, 33.1, 29.6, 28.9, 28.1, 27.3, 22.8.

HRMS (ESI): calculated for C₂₃H₃₄NO₄⁺ ([M+H]⁺) 388.2482, found 388.2474.

[α]_{D}²⁷=−50.9(c0.6,MeOH)

### (2S,3R)-3-hydroxy-2-((2E,4E)-11-methyldodeca-2,4-dienamido)-3-phenylpropanoic acid (S31b)

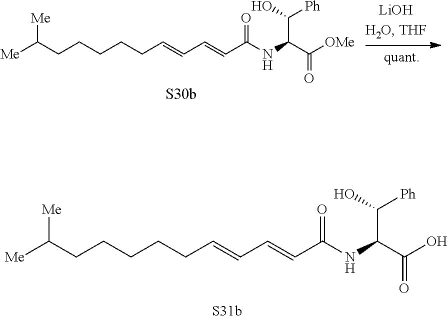 [SMILES: COC(=O)[C@@H](NC(=O)/C=C/C=C/CCCCCC(C)C)[C@H](O)c1ccccc1 ; CC(C)CCCCC/C=C/C=C/C(=O)N[C@H](C(=O)O)[C@H](O)c1ccccc1]

Following General Procedure J: S30b (88 mg, 0.227 mmol, 1 equiv), LiOH (11 mg, 0.454 mmol, 2 equiv) and 22 mL of 1:1 THF:H₂O reaction solvent were used to provide S31b (95 mg, quant.) as a clear, colorless resin.

¹H NMR (400 MHz, CDCl₃): δ 7.37-7.26 (m, 5H), 7.14-7.02 (m, 1H), 6.61 (d, J=7.8 Hz, 1H), 6.13-5.96 (m, 2H), 5.72 (d, J=15.0 Hz, 1H), 5.46 (d, J=2.9 Hz, 1H), 4.91 (dd, J=7.7, 2.9 Hz, 1H), 2.18-2.08 (m, 2H), 1.56-1.45 (m, 1H), 1.38 (p, J=6.6 Hz, 2H), 1.29-1.22 (m, 5H), 1.18-1.11 (m, 2H), 0.86 (d, J=6.6 Hz, 6H).

¹³C NMR (101 MHz, CDCl₃): δ 173.0, 168.4, 145.3, 143.8, 139.0, 128.7, 128.2, 128.1, 125.9, 119.9, 72.2, 58.5, 39.0, 33.2, 29.6, 28.9, 28.1, 27.3, 22.8.

HRMS (ESI): calculated for C₂₂H₃₂NO₄⁺ ([M+H]⁺) 374.2326, found 374.2327.

[α]_{D}²⁷=−29.2(c0.4,MeOH)

### (2E,4E)-N-((1R,2S)-1-hydroxy-3-(((5S,8S,10S,E)-10-hydroxy-5-methyl-2,7-dioxo-1,6-diazacyclododec-3-en-8-yl)amino)-3-oxo-1-phenylpropan-2-yl)-11-methyldodeca-2,4-dienamide (45)

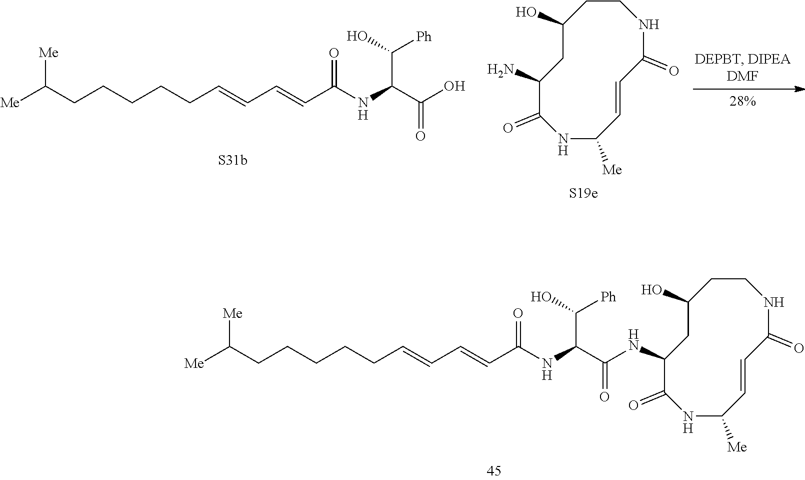 [SMILES: CC(C)CCCCC/C=C/C=C/C(=O)N[C@H](C(=O)O)[C@H](O)c1ccccc1 ; C[C@H]1/C=C/C(=O)NCC[C@H](O)C[C@H](N)C(=O)N1 ; CC(C)CCCCC/C=C/C=C/C(=O)N[C@H](C(=O)N[C@H]1C[C@@H](O)CCNC(=O)/C=C/[C@H](C)NC1=O)[C@H](O)c1ccccc1]

Compound S19e was synthesized as previously described.^{S6} Following General Procedure F: S31b (43 mg, 0.116 mmol, 2 equiv) and S19e (70 mg, 0.0580 mmol as judged by ¹H NMR analysis, 1 equiv) were used to provide 45 (9.7 mg, 28%) as a fluffy white powder.

¹H NMR (600 MHz, DMSO-d₆): δ 8.87-8.42 (m, 1H), 8.01 (d, J=9.2 Hz, 1H), 7.71 (d, J=8.3 Hz, 1H), 7.42 (t, J=6.4 Hz, 1H), 7.35 (d, J=7.4 Hz, 2H), 7.25 (t, J=7.6 Hz, 2H), 7.20-7.15 (m, 1H), 6.84 (dd, J=15.2, 10.8 Hz, 1H), 6.42 (d, J=15.7 Hz, 1H), 6.19 (d, J=15.8 Hz, 1H), 6.16-5.99 (m, 3H), 5.72 (d, J=4.7 Hz, 1H), 5.09 (t, J=3.6 Hz, 1H), 4.70 (s, 1H), 4.55 (dd, J=9.3, 3.0 Hz, 1H), 4.41 (br s, 2H), 3.57 (s, 1H), 3.09-2.93 (m, 2H), 2.10 (q, J=7.2 Hz, 2H), 1.86 (br s, 1H), 1.72-1.55 (m, 1H), 1.54-1.41 (m, 2H), 1.41-1.32 (m, 3H), 1.28-1.18 (m, 7H), 1.16-1.10 (m, 2H), 0.84 (d, J=6.6 Hz, 6H).

¹³C NMR (151 MHz, DMSO-d₆): δ 170.8, 168.9, 167.7, 165.3, 143.0, 142.5, 142.1, 139.7, 128.5, 127.6, 126.9, 126.2, 123.3, 122.9, 72.3, 67.2, 58.7, 51.2, 44.8, 42.9, 38.4, 32.2, 28.8, 28.4, 27.4, 26.6, 22.5, 18.5.

HRMS (ESI): calculated for C₃₃H₄₉N₄O₆⁺ ([M+H]⁺) 597.3647, found 597.3639.

[α]_{D}²⁷=−94.6(c0.1,MeOH)

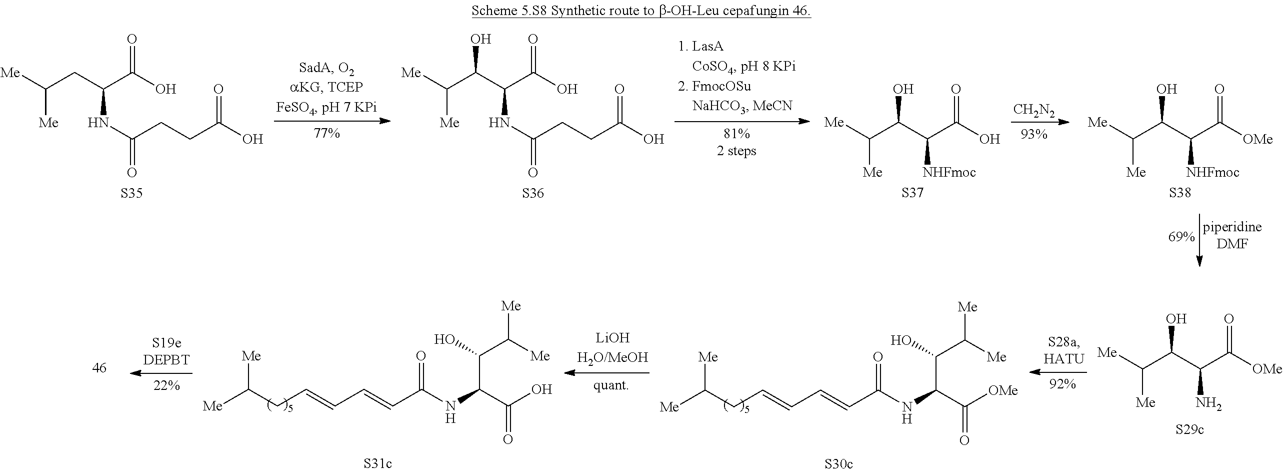

### (2S,3R)-2-(3-carboxypropanamido)-3-hydroxy-4-methylpentanoic Acid (S36)

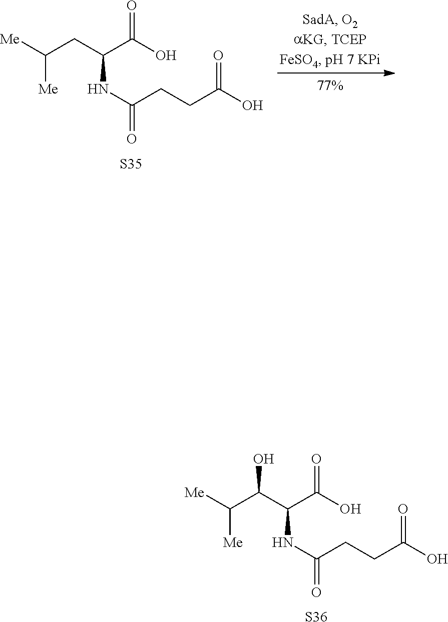 [SMILES: CC(C)C[C@H](NC(=O)CCC(=O)O)C(=O)O ; "S35" ; CC(C)[C@@H](O)[C@H](NC(=O)CCC(=O)O)C(=O)O ; "S36"]

Compound S35 was prepared from L-leucine as described in the literature.^{S10} Following a procedure adapted from Hibi et al.,^{S11} a glycerol stock of E. coli BL21(DE3) cells harboring pET-28a(+)-SadA plasmid was used to inoculate an overnight culture of LB media (6 mL) containing 50 μg/mL kanamycin. 1.0 mL of this culture was used to inoculate each of 4×200 mL TB media containing 50 μg/mL kanamycin in a 1 L non-beveled Erlenmeyer flask. The cultures were shaken at 250 rpm/37° C. for 2.5 h or until an OD₆₀₀=0.6 was reached. The culture was cooled on ice (15 min), induced by adding IPTG to final concentration of 25 μM, then allowed to continue shaking at 250 rpm/23° C. for another 21 h. Cells were harvested by centrifugation (4° C., 15 min, 4200 rpm), then resuspended in 50 mM pH=7 KPi buffer (ca. 45 mL per 200 mL culture) to a final OD₆₀₀=30. The cell suspension was lysed in 45 mL batches by sonication at 50% amplitude for 5 min (1 s on, 4 s off) in an ice bath. The cell debris was pelleted by centrifugation (4° C., 15 min, 4200 rpm), and the clarified lysate supernatant was diluted 1:1 with 50 mM pH=7 KPi and added to a non-beveled Erlenmeyer flask (≥80% headspace) containing S35 (833 mg, 3.6 mmol, 1 equiv, 10 mM final concentration), α-ketoglutaric acid (1.22 g, 5.4 mmol, 1.5 equiv), TCEP (206 mg, 0.720 mmol, 0.2 equiv) and FeSO₄·7H₂O (200 mg, 0.720 mmol, 0.2 equiv). The reaction mixture was then shaken at 250 rpm/30° C. in an open Erlenmeyer flask overnight or until completion by LCMS. The reaction was then quenched by addition of 6 M HCl to final pH=2 and centrifuged (4° C., 15 min, 4200 RPM). The supernatant was concentrated in vacuo to a final volume of ˜25 mL and centrifuged. The resulting pellet was resuspended in ˜5 mL H₂O, centrifuged, and the supernatants were combined and purified by C18 flash chromatography (gradient 0-50% MeOH in H₂O over 10 column volumes, then 3 column volumes to 100% MeOH) to provide S36 (681 mg, 77%) as an off-white solid after lyophilization.

¹H NMR (400 MHz, DMSO-d₆): δ 12.24 (br s, 2H), 7.76 (d, J=9.1 Hz, 1H), 4.88 (br s, 1H), 4.44 (dd, J=9.1, 2.7 Hz, 1H), 3.51 (dd, J=8.9, 2.6 Hz, 1H), 2.48-2.31 (m, 4H), 1.64-1.49 (m, 1H), 0.91 (d, J=6.6 Hz, 3H), 0.76 (d, J=6.7 Hz, 3H).

¹³C NMR (101 MHz, DMSO-d₆): δ 173.8, 172.9, 171.4, 76.2, 54.4, 30.7, 29.9, 29.2, 19.2, 18.9.

HRMS (ESI): calculated for C₁₀H₁₈NO₆⁺ ([M+H]⁺) 248.1129, found 248.1136.

[α]_{D}²⁷=+20.4(c1.6,MeOH)

### (2S,3R)-2-((((9H-fluoren-9-yl)methoxy)carbonyl)amino)-3-hydroxy-4-methylpentanoic Acid (S37)

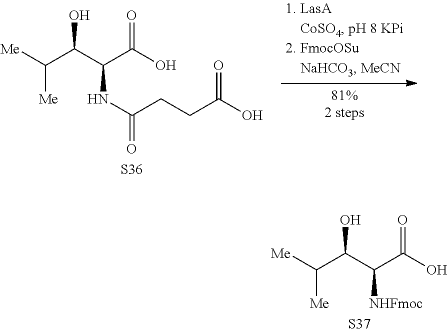

Following a procedure adapted from Hibi et al.,^{S11} a glycerol stock of E. coli BL21(DE3) cells harboring pET-28a(+)-LasA plasmid was used to inoculate an overnight culture of LB media (4 mL) containing 50 μg/mL kanamycin. 0.5 mL of this culture was used to inoculate each of 2×100 mL TB media containing 50 μg/mL kanamycin in 2×500 mL non-beveled Erlenmeyer flasks. The cultures were shaken at 250 rpm/37° C. for 2.5 h or until an OD₆₀₀=0.6 was reached. The culture was cooled on ice (15 min), induced by adding IPTG to final concentration of 25 μM, then allowed to continue shaking at 250 rpm/23° C. for another 20 h. Cells were harvested by centrifugation (4° C., 15 min, 4200 rpm), then resuspended in 50 mM pH=8 KPi buffer (ca. 50 mL per 100 mL culture) to a final OD₆₀₀=20. The combined cell suspensions were lysed by sonication at 50% amplitude for 2×3 min (1 s on, 4 s off) in an ice bath. The cell debris was pelleted by centrifugation (4° C., 15 min, 4200 rpm), and the clarified lysate supernatant was added to a non-beveled 500 mL Erlenmeyer flask containing S36 (247 mg, 1 mmol, 1 equiv, 10 mM final concentration) and CoSO₄·7H₂O (2.81 mg, 0.0100 mmol, 0.01 equiv). The mixture was shaken at 200 rpm/20° C. overnight or until completion by LCMS. The reaction was then directly treated with 10 mL saturated NaHCO₃ solution and FmocOSu (1.35 g, 4 mmol, 4 equiv) dissolved in 30 mL MeCN and stirred at rt for 4 h or until completion by LCMS. The mixture was centrifuged (4° C., 15 min, 4200 rpm) and the supernatant was concentrated in vacuo to remove MeCN. The aqueous phase was adjusted to pH=1 with 6 M HCl and extracted with 4×50 mL EtOAc. The combined organic layers were washed with 1:1 brine:1 M HCl mixture and dried over Na₂SO₄, filtered and concentrated. The residue was purified by flash chromatography (95:5:1 DCM:MeOH:AcOH) and evaporated twice from toluene to provide S37 (294 mg, 81% over 2 steps).

¹H NMR (400 MHz, Methanol-d₄): δ 7.80 (d, J=7.6 Hz, 2H), 7.70-7.65 (m, 2H), 7.44-7.25 (m, 5H), 4.44-4.36 (m, 3H), 4.24 (t, J=7.0 Hz, 1H), 3.68 (dd, J=9.3, 2.4 Hz, 1H), 1.73-1.62 (m, 1H), 1.02 (d, J=6.6 Hz, 3H), 0.90 (d, J=6.7 Hz, 3H).

¹³C NMR (151 MHz, Methanol-d₄): δ 174.90, 158.81, 145.37, 145.06, 142.60, 142.58, 128.78, 128.77, 128.17, 128.14, 126.26, 126.24, 120.92, 120.90, 78.37, 68.00, 48.42, 32.36, 26.26, 19.62, 19.48.

HRMS (ESI): calculated for C₂₁H₂₄NO₅⁺ ([M+H]⁺) 370.1649, found 370.1646.

[α]_{D}²⁴=+2.4(c0.5,MeOH)

### methyl (2S,3R)-2-((((9H-fluoren-9-yl)methoxy)carbonyl)amino)-3-hydroxy-4-methylpentanoate (S38)

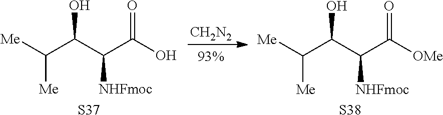

Diazomethane was prepared from N-methyl-N-nitrosourea as described in the literature.^{S12} Compound S37 (50 mg, 0.135 mmol, 1 equiv) was dissolved in 1.4 mL of a 1.5:1 mixture of Et₂O:MeOH and cooled to 0° C. A solution of diazomethane (ca. 10 eq) was added dropwise, and the reaction was stirred at 0° C. for 20 min or until completion by TLC. The reaction was quenched at 0° C. with 0.5 mL AcOH, diluted with 30 mL Et₂O, washed with 2×50 mL NaHCO₃, then brine and dried over Na₂SO₄, filtered and concentrated. The residue was purified by flash chromatography (70:30 Hexanes:EtOAc) to provide S38 (48 mg, 93%) as a colorless oil.

¹H NMR (400 MHz, Methanol-d₄): δ 7.82-7.75 (m, 2H), 7.70-7.62 (m, 2H), 7.45-7.22 (m, 5H), 4.45 (d, J=2.5 Hz, 1H), 4.39 (d, J=6.7 Hz, 2H), 4.22 (t, J=6.9 Hz, 1H), 3.74 (s, 3H), 3.64 (dd, J=9.2, 2.5 Hz, 1H), 1.73-1.60 (m, 1H), 1.01 (d, J=6.6 Hz, 3H), 0.88 (d, J=6.7 Hz, 3H).

¹³C NMR (101 MHz, Methanol-d₄): δ 173.62, 158.78, 145.30, 145.03, 142.61, 142.59, 128.77, 128.16, 128.13, 126.22, 126.19, 120.93, 120.91, 78.17, 68.00, 58.12, 52.81, 48.41, 32.24, 19.56, 19.38.

HRMS (ESI): calculated for C₂₂H₂₆NO₅⁺ ([M+H]⁺) 384.1805, found 384.1807.

[α]_{D}²⁷=−3.7(c0.4,MeOH)

### methyl (2S,3R)-2-amino-3-hydroxy-4-methylpentanoate (S29c)

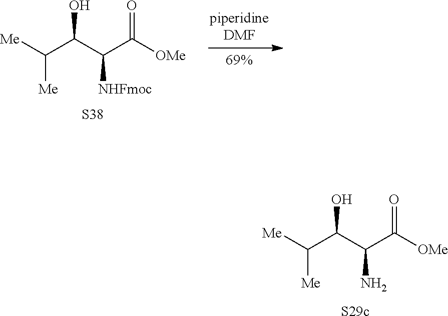

A flame-dried 25 mL reaction vial was charged with S38 (123 mg, 0.321 mmol, 1 equiv) dissolved 6.4 mL anhydrous DMF and cooled to 0° C. Piperidine (1.6 mL, 20% v/v) was added, and the mixture was stirred at 0° C. for 1 hour or until completion by TLC. The mixture was diluted with 10 mL toluene and concentrated to dryness to provide S29c (36 mg, 69%) as a colorless liquid.

¹H NMR (400 MHz, Methanol-d₄): δ 3.73 (s, 3H), 3.57 (d, J=3.1 Hz, 1H), 3.48 (dd, J=8.6, 3.1 Hz, 1H), 1.79 (dhept, J=8.6, 6.7 Hz, 1H), 1.01 (d, J=6.6 Hz, 3H), 0.93 (d, J=6.7 Hz, 3H).

¹³C NMR (101 MHz, Methanol-d₄): δ 176.3, 79.0, 57.6, 52.5, 31.7, 19.7, 19.3.

HRMS (ESI): calculated for C₇H₁₆NO₃⁺ ([M+H]⁺) 162.1125, found 162.1127.

[α]_{D}²⁷=+9.4(c0.5,MeOH)

### methyl (2S,3R)-3-hydroxy-4-methyl-2-((2E,4E)-11-methyldodeca-2,4-dienamido)pentanoate (S30c)

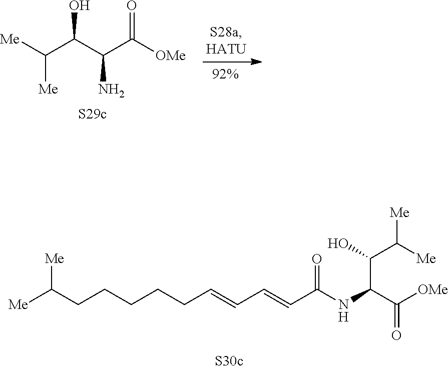 [SMILES: COC(=O)[C@@H](N)[C@H](O)C(C)C ; "S28a,HATU" ; COC(=O)[C@@H](NC(=O)/C=C/C=C/CCCCCC(C)C)[C@H](O)C(C)C]

Following General Procedure K: S29c (29 mg, 0.180 mmol, 1 equiv), S28a (38 mg, 0.180 mmol, 1 equiv), HATU (68 mg, 0.180 mmol, 1 equiv) and DIPEA (31 μL, 0.180 mmol, 1 equiv) were used to provide S30c (59 mg, 92%) as a colorless oil.

¹H NMR (600 MHz, Methanol-d₄): δ 7.16 (dd, J=15.1, 10.8 Hz, 1H), 6.27-6.21 (m, 1H), 6.14 (dt, J=15.0, 6.9 Hz, 1H), 6.06 (d, J=15.2 Hz, 1H), 4.81-4.77 (m, 1H), 3.74 (s, 3H), 3.69 (dd, J=9.0, 2.5 Hz, 1H), 2.21-2.16 (m, 2H), 1.68-1.59 (m, 1H), 1.57-1.50 (m, 1H), 1.45 (p, J=7.2 Hz, 2H), 1.34-1.30 (m, 4H), 1.21-1.17 (m, 2H), 1.01 (d, J=6.6 Hz, 3H), 0.91-0.88 (m, 9H).

¹³C NMR (151 MHz, Methanol-d₄): δ 173.3, 169.3, 144.8, 143.2, 129.8, 122.4, 78.1, 56.4, 52.8, 40.1, 34.0, 32.4, 30.6, 30.0, 29.1, 28.3, 23.0, 19.5, 19.4.

HRMS (ESI): calculated for C₂₀H₃₆NO₄⁺ ([M+H]⁺) 354.2639, found 354.2635.

[α]_{D}²⁷=+10.9(c0.2,MeOH)

### (2S,3R)-3-hydroxy-4-methyl-2-((2E,4E)-11-methyldodeca-2,4-dienamido)pentanoic Acid (S31c)

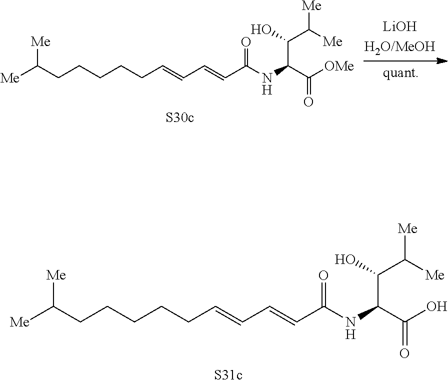 [SMILES: COC(=O)[C@@H](NC(=O)/C=C/C=C/CCCCCC(C)C)[C@H](O)C(C)C ; CC(C)CCCCC/C=C/C=C/C(=O)N[C@H](C(=O)O)[C@H](O)C(C)C]

Following General Procedure J: S30c (42 mg, 0.1189 mmol, 1 equiv) was used to provide S31c (43 mg, quant.) as a colorless resin.

¹H NMR (600 MHz, Methanol-d₄): δ 7.16 (dd, J=15.1, 10.8 Hz, 1H), 6.24 (dd, J=15.1, 10.8 Hz, 1H), 6.16-6.10 (m, 1H), 6.06 (d, J=15.1 Hz, 1H), 4.76 (d, J=2.4 Hz, 1H), 3.73 (dd, J=9.1, 2.4 Hz, 1H), 2.21-2.16 (m, 2H), 1.69-1.60 (m, 1H), 1.57-1.49 (m, 1H), 1.45 (p, J=7.3 Hz, 2H), 1.35-1.30 (m, 4H), 1.21-1.16 (m, 2H), 1.02 (d, J=6.6 Hz, 3H), 0.91 (d, J=6.7 Hz, 3H), 0.88 (d, J=6.6 Hz, 6H).

¹³C NMR (151 MHz, Methanol-d₄): δ 174.6, 169.2, 144.6, 143.1, 129.8, 122.5, 78.2, 56.1, 40.1, 34.0, 32.5, 30.6, 30.0, 29.1, 28.3, 23.0, 19.5, 19.4.

HRMS (ESI): calculated for C₁₉H₃₄NO₄⁺ ([M+H]⁺) 340.2482, found 340.2478.

[α]_{D}²⁷=+28.9(c0.2,MeOH)

### (2E,4E)-N-((2S,3R)-3-hydroxy-1-(((5S,8S,10S,E)-10-hydroxy-5-methyl-2,7-dioxo-1,6-diazacyclododec-3-en-8-yl)amino)-4-methyl-1-oxopentan-2-yl)-11-methyldodeca-2,4-dienamide (46)

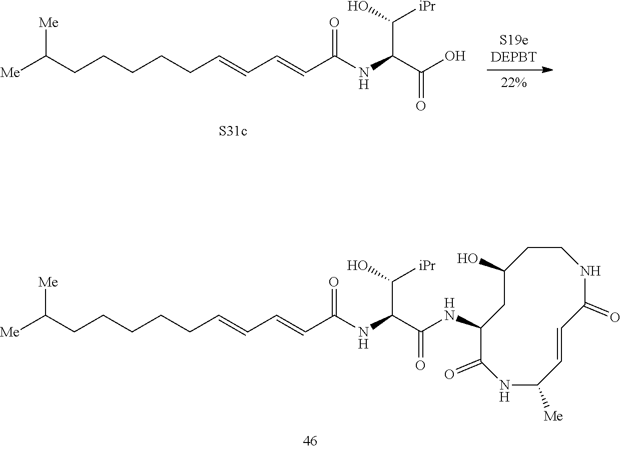 [SMILES: CC(C)CCCCC/C=C/C=C/C(=O)N[C@H](C(=O)O)[C@H](O)C(C)C ; "S19eDEPBT" ; CC(C)CCCCC/C=C/C=C/C(=O)N[C@H](C(=O)N[C@H]1C[C@@H](O)CCNC(=O)/C=C/[C@H](C)NC1=O)[C@H](O)C(C)C]

Following General Procedure F: S31c (31 mg, 0.0913 mmol, 1.5 equiv), S19e (73 mg, 0.0609 mmol as judged by ¹H NMR analysis, 1 equiv), DEPBT (27 mg, 0.0913 mmol, 1.5 equiv) and DIPEA (48 μL, 0.2740, 4.5 equiv) were used to provide 46 (7.6 mg, 22%).

¹H NMR (600 MHz, DMSO-d₆): δ 8.70 (br s, 1H), 7.93 (d, J=9.3 Hz, 1H), 7.60 (d, J=8.1 Hz, 1H), 7.42 (t, J=6.4 Hz, 1H), 7.00 (dd, J=15.1, 10.8 Hz, 1H), 6.40 (d, J=13.4 Hz, 1H), 6.22-6.16 (m, 2H), 6.15-6.07 (m, 2H), 4.82 (d, J=6.7 Hz, 1H), 4.68 (br s, 1H), 4.48 (dd, J=9.4, 2.7 Hz, 1H), 4.38 (br s, 2H), 3.62-3.46 (m, 2H), 3.09-2.92 (m, 2H), 2.13 (q, J=7.2 Hz, 2H), 1.84 (br s, 1H), 1.60 (d, J=13.1 Hz, 1H), 1.53-1.41 (m, 3H), 1.41-1.34 (m, 3H), 1.31-1.17 (m, 7H), 1.16-1.11 (m, 2H), 0.88 (d, J=6.6 Hz, 3H), 0.85 (d, J=6.6 Hz, 6H), 0.77 (d, J=6.7 Hz, 3H).

¹³C NMR (151 MHz, DMSO-d₆): δ 171.0, 170.1, 167.7, 165.4, 143.0, 142.3, 139.9, 128.6, 123.3, 123.0, 76.0, 67.0, 55.2, 51.2, 44.8, 42.6, 38.4, 32.3, 30.8, 28.8, 28.4, 27.4, 26.6, 22.5, 19.1, 19.0, 18.5.

HRMS (ESI): calculated for C₃₀H₅₁N₄O₆⁺ ([M+H]⁺) 563.3803, found 563.3801.

[α]_{D}²⁷=−75.0(c0.1,MeOH)

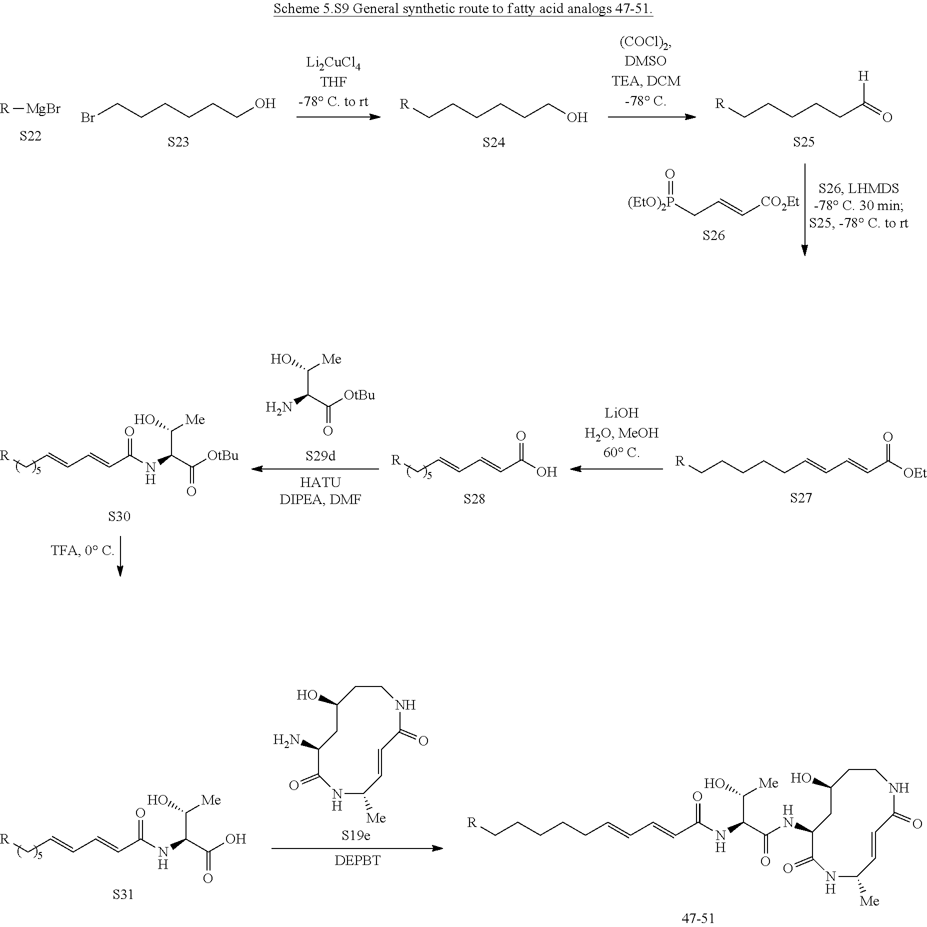

### Synthesis of Tert-Butyl Cepafungin (47)

### 7,7-dimethyloctan-1-ol (S24a)

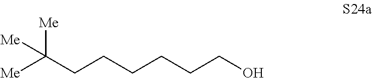 [SMILES: CC(C)(C)CCCCCCO]

Following General Procedure G: tert-butylmagnesium chloride S22a was used to provide S24a (3.90 g, 89%) as a colorless oil.

¹H NMR (400 MHz, CDCl₃): δ 3.63 (t, J=6.7 Hz, 2H), 1.62-1.51 (m, 2H), 1.42-1.10 (m, 9H), 0.85 (s, 9H).

¹³C NMR (101 MHz, CDCl₃): δ 63.2, 44.3, 32.9, 30.5, 30.4, 29.5, 25.9, 24.6.

### 7,7-dimethyloctanal (S25a)

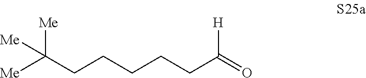 [SMILES: CC(C)(C)CCCCCC=O]

Following General Procedure H: S24a (2.0 g, 12.6 mmol, 1 equiv) was used to provide S25a (2.59 g), used without further purification.

¹H NMR (400 MHz, CDCl₃): δ 9.76 (t, J=1.9 Hz, 1H), 2.42 (td, J=7.4, 1.9 Hz, 2H), 1.70-1.57 (m, 2H), 1.33-1.13 (m, 6H), 0.86 (s, 9H).

### Ethyl (2E,4E)-11,11-dimethyldodeca-2,4-dienoate (S27a)

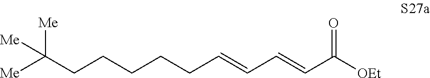 [SMILES: CCOC(=O)/C=C/C=C/CCCCCC(C)(C)C]

Following General Procedure I. S25a (2.59 g) was used to provide S27a (1.75 g, 55% over 2 steps) as a colorless oil.

¹H NMR (400 MHz, CDCl₃): δ 7.31-7.20 (m, 1H), 6.21-6.07 (m, 2H), 5.77 (d, J=15.5 Hz, 1H), 4.19 (q, J=7.1 Hz, 2H), 2.16 (td, J=7.3, 5.8 Hz, 2H), 1.49-1.37 (m, 2H), 1.31-1.22 (m, 7H), 1.18-1.11 (m, 2H), 0.85 (s, 9H).

¹³C NMR (101 MHz, CDCl₃): δ 167.5, 145.3, 144.9, 128.5, 119.3, 60.3, 44.3, 33.2, 30.4, 30.3, 29.5, 28.9, 24.5, 14.5.

HRMS (ESI): calculated for C16H2902+([M+H]⁺) 253.2162, found 253.2159.

### (2E,4E)-11,11-dimethyldodeca-2,4-dienoic Acid (S28b)

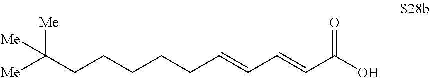 [SMILES: CC(C)(C)CCCCC/C=C/C=C/C(=O)O]

Following General Procedure J: S27a 700 mg, 2.77 mmol, 1 equiv) was used to provide S28b (648 mg, quant.) as a white solid.

¹H NMR (400 MHz, CDCl₃): δ 7.41-7.29 (m, 1H), 6.25-6.14 (m, 2H), 5.78 (d, J=15.3 Hz, 1H), 2.23-2.13 (m, 2H), 1.49-1.40 (m, 2H), 1.31-1.22 (m, 4H), 1.15 (ddt, J=12.2, 5.3, 2.6 Hz, 2H), 0.86 (s, 9H).

¹³C NMR (101 MHz, CDCl₃): δ 172.7, 147.7, 146.5, 128.4, 118.3, 44.3, 33.2, 30.4, 30.3, 29.5, 28.8, 24.5.

HRMS (ESI): calculated for C₁₄H₂₅O₂⁺ ([M+H]⁺) 225.1849, found 225.1849.

### tert-butyl ((2E,4E)-11,11-dimethyldodeca-2,4-dienoyl)-L-threoninate (S30d)

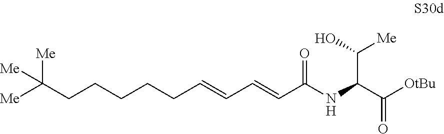 [SMILES: C[C@@H](O)[C@H](NC(=O)/C=C/C=C/CCCCCC(C)(C)C)C(=O)OC(C)(C)C]

Following General Procedure K: S28b (320 mg, 1.43 mmol, 1 equiv), S29d (495 mg, 2.14 mmol, 1.5 equiv), HATU (569 mg, 1.50 mmol, 1.05 equiv) and DIPEA (745 μL, 4.28 mmol, 3 equiv) were used to provide S30d (303 mg, 49%) as a colorless oil. ¹H NMR (400 MHz, CDCl₃): δ 7.21 (dd, J=15.0, 10.3 Hz, 1H), 6.23-6.00 (m, 3H), 5.87 (d, J=15.0 Hz, 1H), 4.49 (dd, J=9.2, 2.1 Hz, 1H), 4.21 (qd, J=6.3, 2.2 Hz, 1H), 2.22-2.07 (m, 2H), 1.49-1.37 (m, 11H), 1.30-1.21 (m, 4H), 1.19-1.11 (m, 14H), 0.85 (s, 9H).

¹³C NMR (101 MHz, CDCl₃): δ 170.2, 166.6, 143.6, 141.9, 128.4, 121.6, 82.0, 73.9, 67.6, 58.4, 44.3, 33.1, 30.4, 30.2, 29.5, 29.0, 28.8, 28.3, 24.5, 21.0.

HRMS (ESI): calculated for C₂₂H₄₀NO₄⁺ ([M+H]⁺) 438.3578, found 438.3573.

[α]_{D}²⁷=+5.3(c0.6,MeOH)

### ((2E,4E)-11,11-dimethyldodeca-2,4-dienoyl)-L-threonine (S31d)

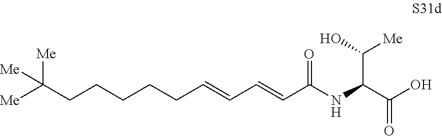 [SMILES: C[C@@H](O)[C@H](NC(=O)/C=C/C=C/CCCCCC(C)(C)C)C(=O)O]

Following General Procedure L: S31d (108 mg, 0.247 mmol, 1 equiv) was used to provide S31d (96 mg, quant.) as a clear, colorless resin.

¹H NMR (400 MHz, CDCl₃): δ 7.25-7.19 (m, 1H), 7.02 (d, J=8.2 Hz, 1H), 6.14 (s, 2H), 5.93 (d, J=15.1 Hz, 1H), 4.68-4.59 (m, 1H), 4.54-4.45 (m, 1H), 2.15 (d, J=6.7 Hz, 2H), 1.46-1.38 (m, 2H), 1.30-1.20 (m, 8H), 1.18-1.12 (m, 2H), 0.86 (s, 9H).

¹³C NMR (101 MHz, CDCl₃): δ 173.6, 168.8, 145.6, 144.0, 128.2, 120.0, 67.7, 57.9, 44.3, 33.3, 30.4, 30.4, 29.6, 29.0, 24.5, 19.3.

HRMS (ESI): calculated for C₁₈H₃₂NO₄⁺ ([M+H]⁺) 326.2326, found 325.2329.

[α]_{D}²⁷=+12.6(c0.3,MeOH)

### (2E,4E)-N-((2S,3R)-3-hydroxy-1-(((5S,8S,10S,E)-10-hydroxy-5-methyl-2,7-dioxo-1,6-diazacyclododec-3-en-8-yl)amino)-1-oxobutan-2-yl)-11,11-dimethyldodeca-2,4-dienamide (47)

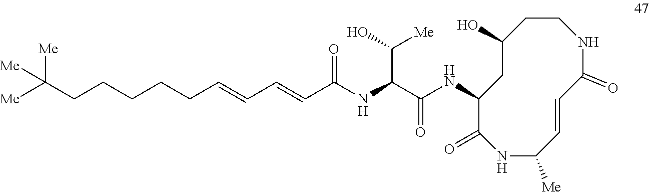 [SMILES: C[C@H]1/C=C/C(=O)NCC[C@H](O)C[C@H](NC(=O)[C@@H](NC(=O)/C=C/C=C/CCCCCC(C)(C)C)[C@@H](C)O)C(=O)N1]

Following General Procedure F: S31d (38 mg, 0.116 mmol, 2 equiv) and S19e (70 mg, 0.0580 mmol as judged by ¹H NMR analysis, 1 equiv) were used to provide 47 (9.6 mg, 30%) as a fluffy white solid.

¹H NMR (600 MHz, DMSO-d₆): δ 8.66 (br s, 1H), 7.90 (d, J=8.7 Hz, 1H), 7.74 (d, J=7.8 Hz, 1H), 7.41 (t, J=6.4 Hz, 1H), 7.00 (dd, J=15.1, 10.8 Hz, 1H), 6.40 (d, J=14.1 Hz, 1H), 6.23-6.05 (m, 4H), 4.86 (d, J=4.9 Hz, 1H), 4.67 (s, 1H), 4.50-4.31 (m, 2H), 4.28 (dd, J=8.8, 4.1 Hz, 1H), 3.96 (s, 1H), 3.57 (s, 1H), 3.07-2.93 (m, 2H), 2.13 (q, J=7.2 Hz, 2H), 1.85 (s, 1H), 1.58 (d, J=13.1 Hz, 1H), 1.49-1.34 (m, 4H), 1.28-1.18 (m, 7H), 1.16-1.11 (m, 2H), 1.00 (d, J=6.3 Hz, 3H), 0.85 (s, 9H).

¹³C NMR (151 MHz, DMSO-d₆): δ 171.0, 169.4, 167.6, 165.4, 143.1, 142.1, 139.7, 128.6, 123.3, 123.1, 67.0, 66.7, 58.1, 51.2, 44.7, 43.7, 42.5, 32.3, 30.1, 29.6, 29.3, 28.4, 23.9, 19.9, 18.5.

HRMS (ESI): calculated for C₂₉H₄₉N₄O₆⁺ ([M+H]⁺) 549.3645, found 549.3645.

[α]_{D}²⁷=−86.4(c0.1,MeOH)

### Synthesis of Cyclopentyl Cepafungin (48)

### 6-cyclopentylhexan-1-ol (S24b)

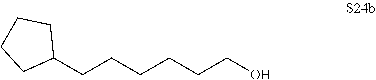 [SMILES: OCCCCCCC1CCCC1]

Following General Procedure G: Cyclopentylmagnesium bromide S22b was used to provide S24b (4.18 g, 89%) as a colorless oil.

¹H NMR (400 MHz, CDCl₃): δ 3.61 (t, J=6.7 Hz, 2H), 1.77-1.66 (m, 3H), 1.65-1.40 (m, 7H), 1.40-1.22 (m, 8H), 1.12-0.95 (m, 2H).

¹³C NMR (101 MHz, CDCl₃): δ 63.1, 40.3, 36.3, 32.9, 32.8, 29.8, 28.9, 25.9, 25.3.

### 6-cyclopentylhexanal (S25b)

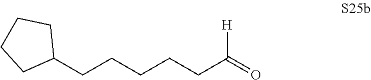 [SMILES: O=CCCCCCC1CCCC1]

Following General Procedure H: S24b (2.50 g, 14.7 mmol, 1 equiv) was used to provide S25b (3.12 g), used without further purification.

¹H NMR (400 MHz, CDCl₃): δ 9.76 (t, J=1.9 Hz, OH), 2.41 (td, J=7.4, 1.9 Hz, 2H), 1.77-1.68 (m, 3H), 1.65-1.45 (m, 6H), 1.35-1.25 (m, 7H), 1.09-1.00 (m, 2H).

### Ethyl (2E,4E)-10-cyclopentyldeca-2,4-dienoate (S27b)

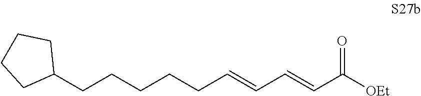 [SMILES: CCOC(=O)/C=C/C=C/CCCCCC1CCCC1]

Following General Procedure I: S25b (3.12 g) was used to provide S27b (1.44 g, 37%) as a colorless oil. ¹H NMR (400 MHz, CDCl₃): δ 7.30-7.19 (m, 1H), 6.22-6.06 (m, 2H), 5.77 (d, J=15.4 Hz, 1H), 4.19 (q, J=7.1 Hz, 2H), 2.15 (td, J=7.4, 5.9 Hz, 2H), 1.78-1.66 (m, 4H), 1.60-1.55 (m, 2H), 1.52-1.46 (m, 2H), 1.45-1.38 (m, 2H), 1.30-1.26 (m, 8H), 1.09-0.99 (m, 2H).

¹³C NMR (101 MHz, CDCl₃): δ 167.5, 145.3, 144.9, 128.4, 119.3, 60.3, 40.2, 36.3, 33.1, 32.8, 29.6, 28.9, 28.7, 25.3, 14.4.

HRMS (ESI): calculated for C17H2902+([M+H]⁺) 265.2162, found 265.2160.

### (2E,4E)-10-cyclopentyldeca-2,4-dienoic Acid (S28c)

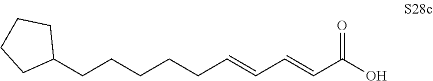 [SMILES: O=C(O)/C=C/C=C/CCCCCC1CCCC1]

Following Genera Procedure J: S27b 718 mg, 2.72 mmol, 1 equiv) was used to provide S28c (645 mg, quant.) as a white solid.

¹H NMR (400 MHz, CDCl₃): δ 7.35 (ddd, J=15.4, 6.8, 3.2 Hz, 1H), 6.25-6.13 (m, 2H), 5.78 (d, J=15.3 Hz, 1H), 2.23-2.13 (m, 2H), 1.78-1.67 (m, 3H), 1.63-1.54 (m, 2H), 1.54-1.47 (m, 2H), 1.47-1.40 (m, 2H), 1.32-1.26 (m, 6H), 1.10-1.00 (m, 2H).

¹³C NMR (101 MHz, CDCl₃): δ 172.9, 147.7, 146.5, 128.3, 118.3, 40.3, 36.3, 33.2, 32.9, 29.6, 28.8, 28.7, 25.3.

HRMS (ESI): calculated for C15H2502+([M+H]⁺) 237.1849, found 237.1847.

### tert-butyl ((2E,4E)-10-cyclopentyldeca-2,4-dienoyl)-L-threoninate (S30e)

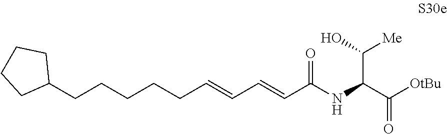 [SMILES: C[C@@H](O)[C@H](NC(=O)/C=C/C=C/CCCCCC1CCCC1)C(=O)OC(C)(C)C]

Following General Procedure K: S28c (350 mg, 1.48 mmol, 1 equiv), S29d (514 mg, 2.22 mmol, 1.5 equiv), HATU (591 mg, 1.56 mmol, 1.05 equiv) and DIPEA (774 μL, 4.44 mmol, 3 equiv) were used to provide S30e (457 mg, 69%) as a colorless oil.

¹H NMR (400 MHz, CDCl₃): δ 7.20 (dd, J=15.0, 10.3 Hz, 1H), 6.20-6.01 (m, 3H), 5.87 (d, J=15.0 Hz, 1H), 4.49 (dd, J=9.2, 2.1 Hz, 1H), 4.21 (qd, J=6.3, 2.2 Hz, 1H), 2.14 (q, J=7.0 Hz, 2H), 1.77-1.67 (m, 3H), 1.62-1.47 (m, 4H), 1.45 (s, 9H), 1.43-1.36 (m, 2H), 1.30-1.24 (m, 6H), 1.18-1.13 (m, 12H), 1.10-0.99 (m, 2H).

¹³C NMR (101 MHz, CDCl₃): δ 170.2, 166.6, 143.6, 141.9, 128.4, 121.6, 82.0, 73.9, 67.6, 58.4, 40.2, 36.3, 33.1, 32.9, 29.6, 29.0, 28.9, 28.7, 28.3, 25.3, 21.0.

HRMS (ESI): calculated for C₂₃H₄₀NO₄⁺ ([M+H]⁺) 450.3578, found 450.3589.

[α]_{D}²⁷=+5.5(c0.4,MeOH)

### ((2E,4E)-10-cyclopentyldeca-2,4-dienoyl)-L-threonine (S31e)

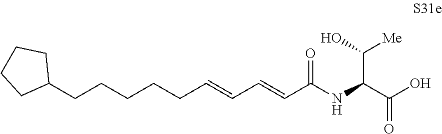 [SMILES: C[C@@H](O)[C@H](NC(=O)/C=C/C=C/CCCCCC1CCCC1)C(=O)O]

Following General Procedure L: S30e (62 mg, 0.138 mmol, 1 equiv) was used to provide S31e (57 mg, quant.) as a clear, colorless resin.

¹H NMR (400 MHz, CDCl₃): δ 7.25-7.14 (m, 1H), 6.88 (d, J=7.9 Hz, 1H), 6.59 (br s, 1H), 6.21-6.06 (m, 2H), 5.91 (d, J=15.0 Hz, 1H), 4.61 (d, J=7.8 Hz, 1H), 4.54-4.43 (m, 1H), 2.21-2.08 (m, 2H), 1.79-1.66 (m, 3H), 1.63-1.45 (m, 4H), 1.45-1.36 (m, 2H), 1.34-1.25 (m, 7H), 1.23 (d, J=6.3 Hz, 3H), 1.10-0.99 (m, 2H).

¹³C NMR (101 MHz, CDCl₃): δ 173.4, 168.6, 145.4, 143.8, 128.2, 120.2, 67.5, 57.8, 40.3, 36.3, 33.3, 32.9, 29.7, 28.9, 28.7, 25.3, 19.2.

HRMS (ESI): calculated for C₁₉H₃₂NO₄⁺ ([M+H]⁺) 338.2326, found 338.2327.

[α]_{D}²⁷=+12.4(c0.3,MeOH)

### (2E,4E)-10-cyclopentyl-N-((2S,3R)-3-hydroxy-1-(((5S,8S,10S,E)-10-hydroxy-5-methyl-2,7-dioxo-1,6-diazacyclododec-3-en-8-yl)amino)-1-oxobutan-2-yl)deca-2,4-dienamide 48)

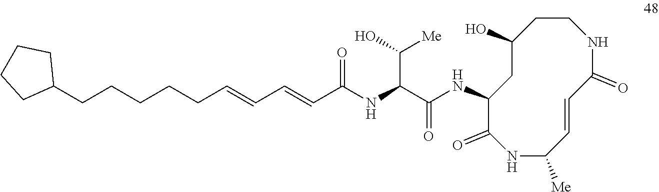 [SMILES: C[C@H]1/C=C/C(=O)NCC[C@H](O)C[C@H](NC(=O)[C@@H](NC(=O)/C=C/C=C/CCCCCC2CCCC2)[C@@H](C)O)C(=O)N1]

Following General Procedure F: S31e (41 mg, 0.121 mmol, 2 equiv) and S19e (73 mg, 0.0603 mmol as judged by ¹H NMR analysis, 1 equiv) were used to provide 48 (12.7 mg, 38%) as a fluffy white solid.

¹H NMR (600 MHz, DMSO-d₆): δ 8.77-8.40 (m, 1H), 7.92 (d, J=8.7 Hz, 1H), 7.84-7.64 (m, 1H), 7.41 (t, J=6.4 Hz, 1H), 7.00 (dd, J=15.2, 10.7 Hz, 1H), 6.40 (d, J=15.6 Hz, 1H), 6.23-6.04 (m, 4H), 4.89 (s, 1H), 4.68 (s, 1H), 4.51-4.31 (m, 2H), 4.28 (dd, J=8.9, 4.1 Hz, 1H), 3.96 (s, 1H), 3.57 (s, 1H), 3.12-2.87 (m, 2H), 2.13 (q, J=7.1 Hz, 2H), 1.84 (br s, 1H), 1.75-1.66 (m, 3H), 1.63-1.50 (m, 3H), 1.50-1.41 (m, 3H), 1.41-1.33 (m, 3H), 1.32-1.23 (m, 7H), 1.23-1.18 (m, 2H), 1.07-1.02 (m, 2H), 1.00 (d, J=6.3 Hz, 3H).

¹³C NMR (151 MHz, DMSO-d₆): δ 171.1, 169.4, 167.6, 165.4, 143.1, 142.1, 139.7, 128.6, 123.3, 123.1, 67.0, 66.7, 58.1, 51.2, 44.7, 42.5, 35.6, 32.3, 28.9, 28.4, 28.0, 24.7, 19.9, 18.5.

HRMS (ESI): calculated for C₃₀H₄₉N₄O₆⁺ ([M+H]⁺) 561.3647, found 561.3655.

[α]_{D}²⁷=−98.4(c0.2,MeOH)

### Synthesis of Cyclohexyl Cepafungin (49)

### 6-cyclohexylhexan-1-ol (S24c)

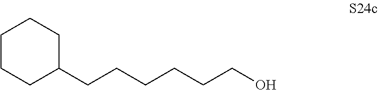 [SMILES: OCCCCCCC1CCCCC1]

Following General Procedure G: Cyclohexylmagnesium bromide S22c was used to provide S24c (4.58 g, 90%) as a colorless oil.

¹H NMR (400 MHz, CDCl₃): δ 3.61 (t, J=6.7 Hz, 2H), 1.73-1.59 (m, 6H), 1.58-1.50 (m, 2H), 1.38-1.23 (m, 6H), 1.23-1.05 (m, 6H), 0.90-0.77 (m, 2H).

¹³C NMR (101 MHz, CDCl₃): δ 63.1, 37.8, 37.6, 33.6, 32.9, 29.9, 26.9, 26.9, 26.6, 25.9.

### 6-cyclohexylhexanal (S25c)

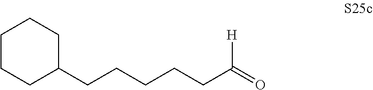 [SMILES: O=CCCCCCC1CCCCC1]

Following General Procedure H: S24c (2.50 g, 13.6 mmol, 1 equiv) was used to provide S25c (3.30 g), used without further purification.

¹H NMR (400 MHz, CDCl₃): δ 9.76 (t, J=1.9 Hz, 1H), 2.41 (td, J=7.4, 1.9 Hz, 2H), 1.71-1.58 (m, 8H), 1.35-1.26 (m, 5H), 1.18-1.12 (m, 4H), 0.85-0.82 (m, 2H).

### Ethyl (2E,4E)-10-cyclohexyldeca-2,4-dienoate (S27c)

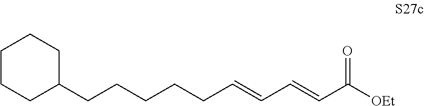 [SMILES: CCOC(=O)/C=C/C=C/CCCCCC1CCCCC1]

Following General Procedure I: S25c (3.30 g) was used to provide S27c (1.62 g, 43% over 2 steps) as a colorless oil.

¹H NMR (400 MHz, CDCl₃): δ 7.30-7.21 (m, 1H), 6.22-6.05 (m, 2H), 5.77 (d, J=15.3 Hz, 1H), 4.19 (q, J=7.1 Hz, 2H), 2.15 (td, J=7.5, 6.0 Hz, 2H), 1.72-1.62 (m, 5H), 1.46-1.37 (m, 2H), 1.28 (t, J=7.1 Hz, 7H), 1.23-1.12 (m, 6H), 0.90-0.79 (m, 2H).

¹³C NMR (101 MHz, CDCl₃): δ 167.5, 145.3, 144.9, 128.4, 119.3, 60.3, 37.8, 37.5, 33.6, 33.1, 29.6, 28.9, 26.9, 26.8, 26.6, 14.5.

HRMS (ESI): calculated for C₁₈H₃₁O₂⁺ ([M+H]⁺) 279.2319, found 279.2318.

### (2E,4E)-10-cyclohexyldeca-2,4-dienoic Acid (S28d)

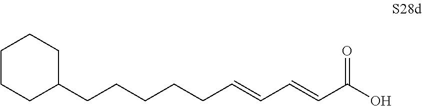 [SMILES: O=C(O)/C=C/C=C/CCCCCC1CCCCC1]

Following General Procedure J: S27c (800 mg, 2.87 mmol, 1 equiv) was used to provide S28d (717 mg, quant.) as a white solid. ¹H NMR (400 MHz, CDCl₃): δ 7.35 (ddd, J=15.3, 6.8, 3.2 Hz, 1H), 6.26-6.12 (m, 2H), 5.78 (d, J=15.3 Hz, 1H), 2.23-2.13 (m, 2H), 1.73-1.59 (m, 5H), 1.43 (s, 2H), 1.32-1.24 (m, 4H), 1.24-1.13 (m, 6H), 0.91-0.79 (m, 2H).

¹³C NMR (101 MHz, CDCl₃): δ 172.8, 147.6, 146.4, 128.2, 118.2, 37.6, 37.4, 33.4, 33.1, 29.5, 28.7, 26.8, 26.6, 26.5.

HRMS (ESI): calculated for C16H2702+([M+H]⁺) 251.2006, found 251.2004.

### tert-butyl ((2E,4E)-10-cyclohexyldeca-2,4-dienoyl)-L-threoninate (S30f)

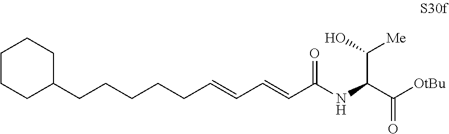 [SMILES: C[C@@H](O)[C@H](NC(=O)/C=C/C=C/CCCCCC1CCCCC1)C(=O)OC(C)(C)C]

Following General Procedure K: S28d (350 mg, 1.40 mmol, 1 equiv), S29d (485 mg, 2.10 mmol, 1.5 equiv), HATU (558 mg, 1.47 mmol, 1.05 equiv) and DIPEA (731 μL, 4.19 mmol, 3 equiv) were used to provide S30f (582 mg, 90%).

¹H NMR (400 MHz, CDCl₃): δ 7.20 (dd, J=15.0, 10.4 Hz, 1H), 6.22-5.99 (m, 3H), 5.87 (d, J=15.0 Hz, 1H), 4.49 (dd, J=9.2, 2.1 Hz, 1H), 4.21 (qd, J=6.2, 2.2 Hz, 1H), 2.14 (q, J=7.1 Hz, 2H), 1.71-1.59 (m, 5H), 1.45 (s, 9H), 1.43-1.35 (m, 2H), 1.30-1.20 (m, 5H), 1.20-1.11 (m, 17H), 0.90-0.78 (m, 2H).

¹³C NMR (101 MHz, CDCl₃): δ 170.2, 166.6, 143.6, 141.9, 128.4, 121.6, 82.0, 73.9, 67.6, 58.4, 37.8, 37.6, 33.6, 33.1, 29.6, 29.0, 28.8, 28.2, 26.9, 26.8, 26.6, 21.0.

HRMS (ESI): calculated for C₂₄H₄₂NO₄⁺ ([M+H]⁺) 464.3734, found 464.3733.

[α]_{D}²⁷=+5.5(c0.7,MeOH)

### ((2E,4E)-10-cyclohexyldeca-2,4-dienoyl)-L-threonine (S31f)

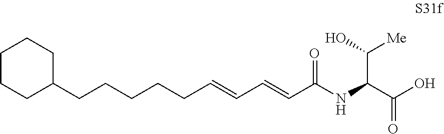 [SMILES: C[C@@H](O)[C@H](NC(=O)/C=C/C=C/CCCCCC1CCCCC1)C(=O)O]

Following General Procedure L: S30f (60 mg, 0.129 mmol, 1 equiv) was used to provide S31f (61 mg, quant.).

¹H NMR (400 MHz, CDCl₃): δ 7.26-7.15 (m, 1H), 6.87 (d, J=8.0 Hz, 1H), 6.32 (br s, 1H), 6.19-6.07 (m, 2H), 5.91 (d, J=15.0 Hz, 1H), 4.61 (dd, J=8.1, 2.3 Hz, 1H), 4.55-4.40 (m, 1H), 2.15 (q, J=6.7 Hz, 2H), 1.73-1.59 (m, 5H), 1.45-1.36 (m, 2H), 1.33-1.20 (m, 8H), 1.20-1.12 (m, 5H), 0.91-0.79 (m, 2H).

¹³C NMR (101 MHz, CDCl₃): δ 173.4, 168.5, 145.3, 143.7, 128.2, 120.2, 67.5, 57.8, 37.8, 37.6, 33.6, 33.3, 29.7, 29.0, 26.9, 26.8, 26.6, 19.2.

HRMS (ESI): calculated for C₂₀H₃₄NO₄⁺ ([M+H]⁺) 352.2482, found 352.2483.

[α]_{D}²⁷=+13.4(c0.4,MeOH)

### (2E,4E)-10-cyclohexyl-N-((2S,3R)-3-hydroxy-1-(((5S,8S,10S,E)-10-hydroxy-5-methyl-2,7-dioxo-1,6-diazacyclododec-3-en-8-yl)amino)-1-oxobutan-2-yl)deca-2,4-dienamide (49)

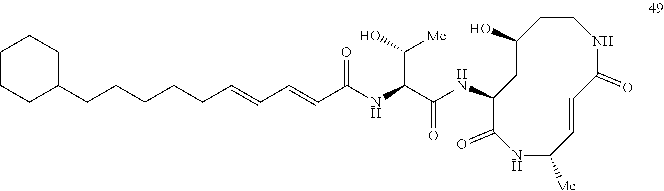 [SMILES: C[C@H]1/C=C/C(=O)NCC[C@H](O)C[C@H](NC(=O)[C@@H](NC(=O)/C=C/C=C/CCCCCC2CCCCC2)[C@@H](C)O)C(=O)N1]

Following General Procedure F: S31f (49 mg, 0.139 mmol, 2 equiv) and S19e (84 mg, 0.0693 mmol as judged by ¹H NMR analysis, 1 equiv) were used to provide 49 (16.5 mg, 41%) as a fluffy white solid.

¹H NMR (600 MHz, DMSO-d₆): δ 8.85-8.35 (m, 1H), 7.97-7.85 (m, 1H), 7.82-7.65 (m, 1H), 7.41 (t, J=6.3 Hz, 1H), 7.00 (dd, J=15.1, 10.8 Hz, 1H), 6.40 (d, J=14.6 Hz, 1H), 6.24-6.02 (m, 4H), 4.88 (s, 1H), 4.68 (s, 1H), 4.51-4.30 (m, 2H), 4.28 (dd, J=8.8, 4.1 Hz, 1H), 4.03-3.88 (m, 1H), 3.64-3.51 (m, 1H), 3.13-2.85 (m, 2H), 2.12 (q, J=7.2 Hz, 2H), 1.96-1.73 (m, 1H), 1.69-1.54 (m, 6H), 1.49-1.41 (m, 1H), 1.40-1.34 (m, 3H), 1.29-1.10 (m, 13H), 1.00 (d, J=6.3 Hz, 3H), 0.88-0.78 (m, 2H).

¹³C NMR (151 MHz, DMSO-d₆): δ 171.1, 169.4, 167.6, 165.4, 143.1, 142.1, 139.7, 128.6, 123.3, 123.1, 66.9, 66.7, 58.1, 51.2, 44.7, 42.5, 37.0, 36.9, 32.9, 32.2, 28.9, 28.4, 26.2, 26.1, 25.9, 19.9, 18.5.

HRMS (ESI): calculated for C₃₁H₅₁N₄O₆⁺ ([M+H]⁺) 575.3803, found 575.3808.

[α]_{D}²⁷=−93.1(c0.2,MeOH)

### Synthesis of Phenyl Cepafungin (50)

### 6-phenylhexan-1-ol (S24d)

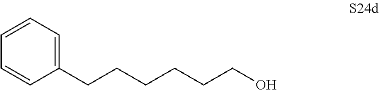 [SMILES: OCCCCCCc1ccccc1]

Following Procedure G: Phenylmagnesium bromide S22d was used to provide S24d (3.95 g, 80%) as a colorless oil.

¹H NMR (400 MHz, CDCl₃): δ 7.34-7.12 (m, 5H), 3.62 (t, J=6.6 Hz, 2H), 2.67-2.55 (m, 2H), 1.68-1.52 (m, 5H), 1.44-1.33 (m, 4H).

¹³C NMR (101 MHz, CDCl₃): δ 142.8, 128.5, 128.4, 125.7, 63.1, 36.0, 32.8, 31.5, 29.2, 25.7.

HRMS (ESI): calculated for C₁₂H₁₉O⁺ ([M+H]⁺) 179.1430, found 179.1430.

### 6-phenylhexanal (S25d)

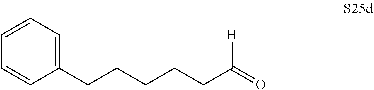 [SMILES: O=CCCCCCc1ccccc1]

Following General Procedure H: S24d (2.00 g, 11.2 mmol, 1 equiv) was used to provide S25d, used immediately without further purification.

¹H NMR (400 MHz, CDCl₃): δ 9.76 (t, J=1.8 Hz, 1H), 7.30-7.25 (m, 2H), 7.20-7.15 (m, 3H), 2.64-2.59 (m, 2H), 2.45-2.40 (m, 2H), 1.71-1.60 (m, 4H), 1.42-1.33 (m, 2H).

### Ethyl (2E,4E)-10-phenyldeca-2,4-dienoate (S27d)

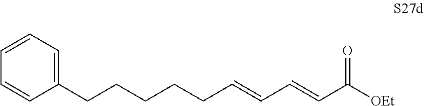 [SMILES: CCOC(=O)/C=C/C=C/CCCCCc1ccccc1]

Following General Procedure I: S25d was used to provide S27d (1.08 g, 35% over 2 steps) as a colorless oil. ¹H NMR (400 MHz, CDCl₃): δ 7.31-7.21 (m, 3H), 7.20-7.15 (m, 3H), 6.21-6.06 (m, 2H), 5.78 (d, J=15.4 Hz, 1H), 4.20 (q, J=7.1 Hz, 2H), 2.65-2.56 (m, 2H), 2.16 (q, J=7.0 Hz, 2H), 1.68-1.58 (m, 2H), 1.51-1.42 (m, 2H), 1.40-1.33 (m, 2H), 1.29 (t, J=7.1 Hz, 3H).

¹³C NMR (101 MHz, CDCl₃): δ 167.5, 145.2, 144.6, 142.8, 128.6, 128.5, 128.4, 125.8, 119.4, 60.3, 36.0, 33.0, 31.4, 28.9, 28.7, 14.5.

HRMS (ESI): calculated for C18H2502+([M+H]⁺) 273.1849, found 273.1848.

### (2E,4E)-10-phenyldeca-2,4-dienoic Acid (S28e)

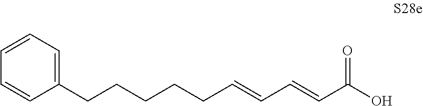 [SMILES: O=C(O)/C=C/C=C/CCCCCc1ccccc1]

Following General Procedure J: S27d (500 mg, 1.84 mmol, 1 equiv) was used to provide S28e (474 mg, quant.) as a white solid.

¹H NMR (400 MHz, CDCl₃): δ 7.38-7.31 (m, 1H), 7.30-7.25 (m, 2H), 7.21-7.15 (m, 3H), 6.24-6.12 (m, 2H), 5.79 (d, J=15.2 Hz, 1H), 2.64-2.58 (m, 2H), 2.18 (q, J=7.0 Hz, 2H), 1.68-1.59 (m, 2H), 1.47 (tt, J=8.0, 6.7 Hz, 2H), 1.40-1.33 (m, 2H).

¹³C NMR (101 MHz, CDCl₃): δ 172.92, 147.65, 146.21, 142.72, 128.51, 128.44, 128.41, 125.80, 118.44, 35.95, 33.09, 31.37, 28.91, 28.61.

HRMS (ESI): calculated for C16H2102+([M+H]⁺) 245.1536, found 245.1535.

### tert-butyl ((2E,4E)-10-phenyldeca-2,4-dienoyl)-L-threoninate (S30g)

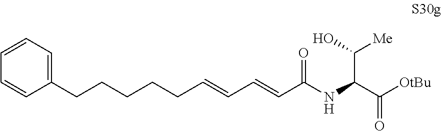 [SMILES: C[C@@H](O)[C@H](NC(=O)/C=C/C=C/CCCCCc1ccccc1)C(=O)OC(C)(C)C]

Following General Procedure K: S28e (237 mg, 0.970 mmol, 1 equiv), S29d (337 mg, 1.46 mmol, 1.5 equiv), HATU (387 mg, 1.02 mmol, 1.05 equiv) and DIPEA (507 μL, 2.91 mmol, 3 equiv) were used to provide S30g (253 mg, 57%) as a colorless oil.

¹H NMR (400 MHz, CDCl₃): δ 7.32-7.21 (m, 2H), 7.22-7.14 (m, 4H), 6.20-6.00 (m, 3H), 5.88 (d, J=15.0 Hz, 1H), 4.50 (dd, J=9.2, 2.1 Hz, 1H), 4.22 (qd, J=6.3, 2.2 Hz, 1H), 2.64-2.57 (m, 2H), 2.15 (q, J=7.0 Hz, 2H), 1.67-1.58 (m, 2H), 1.50-1.41 (m, 11H), 1.39-1.32 (m, 2H), 1.20-1.14 (m, 12H).

¹³C NMR (101 MHz, CDCl₃): δ 170.21, 166.60, 143.26, 142.82, 141.82, 128.53, 128.40, 125.77, 121.78, 81.98, 73.94, 67.60, 58.45, 35.99, 32.97, 31.40, 28.91, 28.87, 28.79, 28.27, 21.01.

HRMS (ESI): calculated for C₂₈H₄₄NO₄⁺ ([M+H]⁺) 458.3265, found 458.3263.

[α]_{D}²⁷=+4.6(c0.4,MeOH)

### ((2E,4E)-10-phenyldeca-2,4-dienoyl)-L-threonine (S31g)

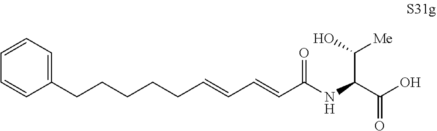 [SMILES: C[C@@H](O)[C@H](NC(=O)/C=C/C=C/CCCCCc1ccccc1)C(=O)O]

Following Procedure L: S30g (101 mg, 0.221 mmol, 1 equiv) was used to provide S31g (93 mg, quant.) as a clear, colorless resin.

¹H NMR (400 MHz, CDCl₃): δ 7.29-7.27 (m, 1H), 7.25-7.14 (m, 5H), 6.91 (d, J=8.0 Hz, 1H), 6.52 (br s, 1H), 6.18-6.05 (m, 2H), 5.91 (d, J=15.0 Hz, 1H), 4.62 (dd, J=8.1, 2.4 Hz, 1H), 4.53-4.44 (m, 1H), 2.63-2.55 (m, 2H), 2.14 (q, J=6.7 Hz, 2H), 1.66-1.57 (m, 2H), 1.49-1.29 (m, 5H), 1.22 (d, J=6.3 Hz, 3H).

¹³C NMR (101 MHz, CDCl₃): δ 173.4, 168.5, 145.1, 143.7, 142.7, 128.5, 128.4, 128.3, 125.8, 120.3, 67.5, 57.8, 36.0, 33.1, 31.4, 29.0, 28.8, 19.3.

HRMS (ESI): calculated for C₂₀H₂₈NO₄⁺ ([M+H]⁺) 346.2013, found 346.2014.

[α]_{D}²⁷=+11.4,(c0.3,MeOH)

### (2E,4E)-N-((2S,3R)-3-hydroxy-1-(((5S,8S,10S,E)-10-hydroxy-5-methyl-2,7-dioxo-1,6-diazacyclododec-3-en-8-yl)amino)-1-oxobutan-2-yl)-10-phenyldeca-2,4-dienamide (50)

Following General Procedure F: S31g (189 mg, 0.546 mmol, 2 equiv) and S19e (70 mg, 0.0580 mmol as judged by ¹H NMR analysis, 1 equiv) were used to provide 50 (44.3 mg, 29%) as a

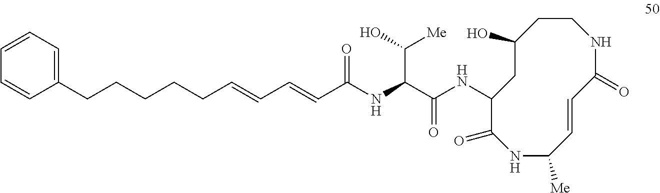 [SMILES: C[C@H]1/C=C/C(=O)NCC[C@H](O)CC(NC(=O)[C@@H](NC(=O)/C=C/C=C/CCCCCc2ccccc2)[C@@H](C)O)C(=O)N1]

fluffy white powder.

¹H NMR (600 MHz, DMSO-d₆): δ 8.75-8.43 (m, 1H), 7.93 (d, J=8.7 Hz, 1H), 7.75 (d, J=6.5 Hz, 1H), 7.41 (t, J=6.2 Hz, 1H), 7.28-7.24 (m, 2H), 7.20-7.14 (m, 3H), 6.99 (dd, J=15.1, 10.8 Hz, 1H), 6.40 (d, J=15.8 Hz, 1H), 6.22-6.05 (m, 4H), 4.89 (br s, 1H), 4.68 (br s, 1H), 4.53-4.31 (m, 2H), 4.28 (dd, J=8.8, 4.1 Hz, 1H), 4.02-3.89 (m, 1H), 3.61-3.53 (m, 1H), 3.07-2.94 (m, 2H), 2.56 (t, J=7.7 Hz, 2H), 2.13 (q, J=7.1 Hz, 2H), 1.95-1.76 (m, 1H), 1.62-1.53 (m, 3H), 1.49-1.34 (m, 4H), 1.33-1.26 (m, 2H), 1.25-1.15 (m, 3H), 1.00 (d, J=6.3 Hz, 3H).

¹³C NMR (151 MHz, DMSO-d₆): δ 171.05, 169.38, 167.63, 165.43, 143.11, 142.26, 142.02, 139.72, 128.62, 128.27, 128.23, 125.61, 123.29, 123.12, 66.95, 66.72, 58.13, 51.19, 44.74, 42.46, 35.07, 32.18, 30.78, 28.19, 28.17, 19.93, 18.55.

HRMS (ESI): calculated for C₃₁H₄₅N₄O₆⁺ ([M+H]⁺) 569.3334, found 569.3338.

[α]_{D}²⁷=−103.3(c0.1,MeOH)

### Synthesis of Trifluoromethyl Cepafungin (51)

### 7,7,7-trifluoroheptan-1-ol (S24e)

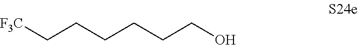 [SMILES: OCCCCCCC(F)(F)F]

Compound S24e was prepared as described by Pitre et al.^{S13}

### 7,7,7-trifluoroheptanal (S25e)

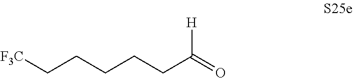 [SMILES: O=CCCCCCC(F)(F)F]

Following General Procedure H: S24e (308 mg, 1.81 mmol, 1 equiv) was used to provide S25e, used immediately without further purification.

¹H NMR (400 MHz, CDCl₃): δ 9.76 (t, J=1.6 Hz, 1H), 2.52-2.38 (m, 2H), 2.22-1.99 (m, 2H), 1.82-1.49 (m, 4H), 1.45-1.22 (m, 2H).

HRMS (ESI): calculated for C₇H₁₂F₃O⁺ ([M+H]⁺) 169.0835, found 169.0843.

### Ethyl (2E,4E)-11,11,11-trifluoroundeca-2,4-dienoate (S27e)

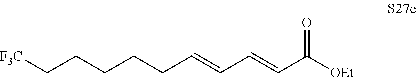 [SMILES: CCOC(=O)/C=C/C=C/CCCCCC(F)(F)F]

Following General Procedure I. S25e was used to provide S27e (120 mg, 25% over 2 steps).

¹H NMR (400 MHz, CDCl₃): δ 7.25 (dd, J=15.4, 10.3 Hz, 1H), 6.23-6.02 (m, 2H), 5.79 (d, J=15.5 Hz, 1H), 4.19 (q, J=7.1 Hz, 2H), 2.18 (q, J=7.0 Hz, 2H), 2.14-1.95 (m, 2H), 1.62-1.51 (m, 2H), 1.50-1.32 (m, 4H), 1.29 (t, J=7.1 Hz, 3H).

¹³C NMR (101 MHz, CDCl₃): δ 167.4, 144.9, 143.9, 128.9, 119.7, 60.3, 53.6, 32.8, 33.8 (q, J=28.4 Hz), 28.4, 28.3, 21.8 (q, J=2.9 Hz), 14.4.

HRMS (ESI): calculated for C₁₃H₂₀F₃O₂⁺ ([M+H]⁺) 265.1410, found 265.1409.

### (2E,4E)-11,11,11-trifluoroundeca-2,4-dienoic Acid (S28f)

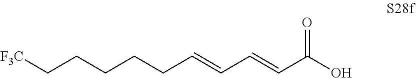 [SMILES: O=C(O)/C=C/C=C/CCCCCC(F)(F)F]

Following General Procedure J: S27e (95 mg, 0.359 mmol, 1 equiv) was used to provide S28f (77 mg, 91%).

¹H NMR (600 MHz, CDCl₃): δ 7.34 (dd, J=15.3, 10.3 Hz, 1H), 6.24-6.13 (m, 2H), 5.80 (d, J=15.3 Hz, 1H), 2.23-2.18 (m, 2H), 2.12-2.01 (m, 2H), 1.62-1.52 (m, 2H), 1.52-1.44 (m, 2H), 1.43-1.35 (m, 2H).

¹³C NMR (151 MHz, CDCl₃): δ 172.7, 147.4, 145.5, 128.7, 118.7, 33.8 (q, J=28.4 Hz), 32.8, 28.3, 21.9 (q, J=2.9 Hz).

HRMS (ESI): calculated for C₁₁H₁₆F₃O₂⁺ ([M+H]⁺) 237.1097, found 237.1089.

### tert-butyl ((2E,4E)-11,11,11-trifluoroundeca-2,4-dienoyl)-L-threoninate (S30h)

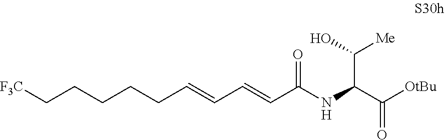 [SMILES: C[C@@H](O)[C@H](NC(=O)/C=C/C=C/CCCCCC(F)(F)F)C(=O)OC(C)(C)C]

Following General Procedure K: S28f (66 mg, 0.279 mmol, 1 equiv), S29d (97 mg, 0.419 mmol, 1.5 equiv), HATU (112 mg, 0.293 mmol, 1.05 equiv) and DIPEA (49 μL, 0.279 mmol, 1 equiv) were used to provide S30h (98 mg, 78%).

¹H NMR (600 MHz, CDCl₃): δ 7.21 (dd, J=15.0, 10.8 Hz, 1H), 6.22-6.10 (m, 2H), 6.04 (dt, J=14.7, 6.9 Hz, 1H), 5.89 (d, J=15.0 Hz, 1H), 4.49 (dd, J=9.2, 2.1 Hz, 1H), 4.21 (qd, J=6.2, 2.1 Hz, 1H), 2.21-2.14 (m, 2H), 2.12-1.99 (m, 2H), 1.61-1.51 (m, 2H), 1.51-1.41 (m, 11H), 1.41-1.33 (m, 2H), 1.20-1.14 (m, 12H).

¹³C NMR (151 MHz, CDCl₃): δ 170.2, 166.5, 142.6, 141.7, 128.8, 122.0, 82.0, 73.9, 67.6, 58.5, 33.8 (q, J=28.4 Hz), 32.7, 28.9, 28.5, 28.3, 28.3, 21.8 (q, J=2.9 Hz), 21.0.

HRMS (ESI): calculated for C₂₃H₃₉F₃NO₄⁺ ([M+H]⁺) 450.2826, found 450.2815.

[α]_{D}²⁷=+4.6(c0.3,MeOH)

### ((2E,4E)-11,11,11-trifluoroundeca-2,4-dienoyl)-L-threonine (S31h)

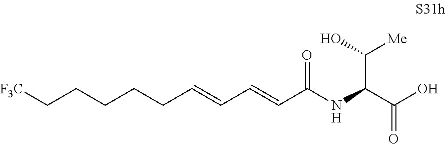 [SMILES: C[C@@H](O)[C@H](NC(=O)/C=C/C=C/CCCCCC(F)(F)F)C(=O)O]

Following General Procedure L: S30h (45 mg, 0.100 mmol, 1 equiv) was used to provide S31h (43 mg, quant.)

¹H NMR (600 MHz, CDCl₃): δ 7.26-7.18 (m, 1H), 6.86 (s, 1H), 6.21-6.06 (m, 2H), 5.93 (d, J=14.8 Hz, 1H), 4.60 (d, J=7.0 Hz, 1H), 4.49 (s, 1H), 2.21-2.14 (m, 2H), 2.12-2.01 (m, 2H), 1.56 (p, J=8.1 Hz, 2H), 1.49-1.42 (m, 2H), 1.41-1.35 (m, 2H), 1.31-1.17 (m, 5H).

¹³C NMR (151 MHz, CDCl₃): δ 173.3, 168.5, 144.4, 143.5, 128.6, 120.5, 67.4, 57.8, 33.8 (q, J=28.3 Hz), 32.8, 29.9, 28.4, 28.3, 21.8 (q, J=2.9 Hz), 19.3.

HRMS (ESI): calculated for C₁₅H₂₃F₃NO₄⁺ ([M+H]⁺) 338.1574, found 338.1583.

[α]_{D}²⁷=+8.1(c0.3,MeOH)

### (2E,4E)-11,11,11-trifluoro-N-((2S,3R)-3-hydroxy-1-(((5S,8S,10S,E)-10-hydroxy-5-methyl-2,7-dioxo-1,6-diazacyclododec-3-en-8-yl)amino)-1-oxobutan-2-yl)undeca-2,4-dienamide (51)

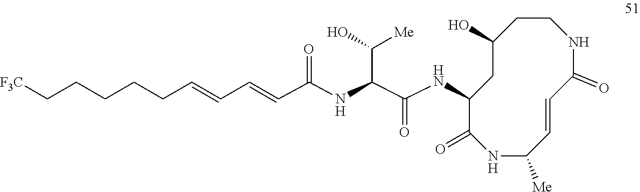 [SMILES: C[C@H]1/C=C/C(=O)NCC[C@H](O)C[C@H](NC(=O)[C@@H](NC(=O)/C=C/C=C/CCCCCC(F)(F)F)[C@@H](C)O)C(=O)N1]

Following General Procedure F: S31h (34 mg, 0.101 mmol, 2 equiv) and S19e (60 mg, 0.0501 mmol as judged by ¹H NMR analysis, 1 equiv) were used to provide 51 (8.9 mg, 32%) as a fluffy white solid.

¹H NMR (600 MHz, DMSO-d₆): δ 8.67 (s, 1H), 7.92 (d, J=8.6 Hz, 1H), 7.74 (d, J=7.8 Hz, 1H), 7.41 (t, J=6.2 Hz, 1H), 7.00 (dd, J=15.1, 10.9 Hz, 1H), 6.40 (d, J=14.4 Hz, 1H), 6.24-6.05 (m, 4H), 4.87 (d, J=4.6 Hz, 1H), 4.67 (s, 1H), 4.49-4.30 (m, 2H), 4.28 (dd, J=8.8, 4.1 Hz, 1H), 4.04-3.88 (m, 1H), 3.57 (s, 1H), 3.08-2.92 (m, 2H), 2.28-2.18 (m, 2H), 2.14 (q, J=6.9 Hz, 2H), 1.85 (s, 1H), 1.58 (d, J=13.2 Hz, 1H), 1.51-1.45 (m, 2H), 1.45-1.38 (m, 3H), 1.38-1.31 (m, 3H), 1.26-1.14 (m, 3H), 1.00 (d, J=6.3 Hz, 3H).

¹³C NMR (151 MHz, DMSO-d₆): δ 171.0, 169.4, 167.6, 165.4, 143.1, 141.8, 139.7, 128.7, 123.3, 123.2, 67.0, 66.7, 58.1, 51.2, 44.8, 42.5, 32.3 (q, J=27.3 Hz), 32.0, 27.9, 27.5, 21.3 (q, J=2.9 Hz), 19.9, 18.5.

HRMS (ESI): calculated for C₂₆H₄₀F₃N₄O₆⁺ ([M+H]⁺) 561.2894, found 561.2901.

[α]_{D}²⁷=−80.6(c0.2,MeOH)

### Synthesis of Hybrid Phenyl-Valinyl Cepafungin (S39)

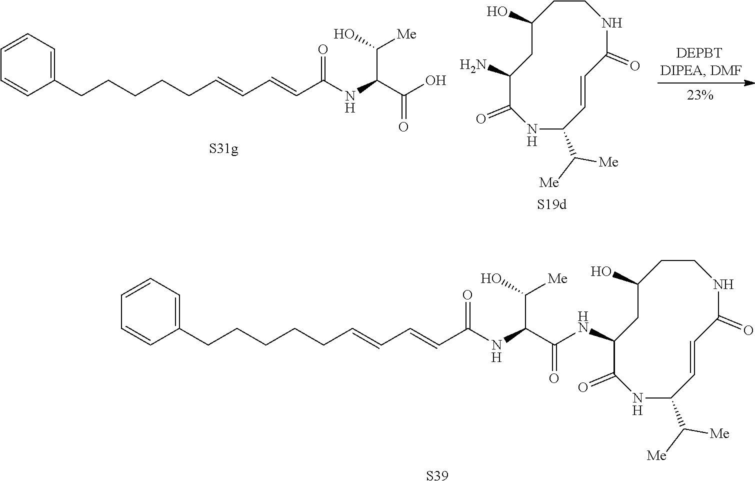 [SMILES: C[C@@H](O)[C@H](NC(=O)/C=C/C=C/CCCCCc1ccccc1)C(=O)O ; CC(C)[C@H]1/C=C/C(=O)NCC[C@H](O)C[C@H](N)C(=O)N1 ; CC(C)[C@H]1/C=C/C(=O)NCC[C@H](O)C[C@H](NC(=O)[C@@H](NC(=O)/C=C/C=C/CCCCCc2ccccc2)[C@@H](C)O)C(=O)N1 ; "S39"]

### (2E,4E)-N-((2S,3R)-3-hydroxy-1-(((5S,8S,10S,E)-10-hydroxy-5-isopropyl-2,7-dioxo-1,6-diazacyclododec-3-en-8-yl)amino)-1-oxobutan-2-yl)-10-phenyldeca-2,4-dienamide (S39)

Following General Procedure F: S31g (38 mg, 0.109 mmol, 2.04 equiv) and S19d (56 mg, 0.0535 mmol as judged by ¹H NMR analysis, 1 equiv) were used to provide S39 (7.5 mg, 23%) as a fluffy white solid.

¹H NMR (600 MHz, DMSO-d₆): δ 8.48 (d, J=8.6 Hz, 1H), 7.92 (d, J=8.7 Hz, 1H), 7.79 (d, J=7.7 Hz, 1H), 7.42 (t, J=6.3 Hz, 1H), 7.29-7.24 (m, 2H), 7.19-7.14 (m, 3H), 6.99 (dd, J=15.1, 10.8 Hz, 1H), 6.33 (dd, J=15.9, 6.6 Hz, 1H), 6.23-6.03 (m, 4H), 4.84 (d, J=5.1 Hz, 1H), 4.67 (d, J=5.0 Hz, 1H), 4.45-4.32 (m, 1H), 4.28 (dd, J=8.8, 4.5 Hz, 1H), 4.10-3.99 (m, 1H), 3.99-3.84 (m, 1H), 3.65-3.49 (m, 1H), 3.05-2.93 (m, 2H), 2.56 (t, J=7.7 Hz, 2H), 2.12 (q, J=7.1 Hz, 2H), 1.86 (td, J=12.3, 6.8 Hz, 1H), 1.75 (dq, J=13.6, 6.9 Hz, 1H), 1.61-1.53 (m, 3H), 1.45-1.35 (m, 4H), 1.32-1.26 (m, 2H), 0.99 (d, J=6.3 Hz, 3H), 0.94-0.86 (m, 6H).

¹³C NMR (151 MHz, DMSO-d₆): δ 171.47, 169.37, 167.77, 165.38, 142.27, 141.98, 140.82, 139.68, 128.64, 128.28, 128.24, 125.62, 124.76, 123.16, 66.88, 66.79, 58.17, 55.64, 51.21, 42.25, 35.09, 32.19, 30.80, 30.58, 28.20, 28.18, 19.77, 19.63, 19.12.

HRMS (ESI): calculated for C₃₃H₄₉N₄O₆⁺ ([M+H]⁺) 597.3647, found 597.3643.

[α]_{D}²⁷=−70.0(c0.1,MeOH)

### SUPPLEMENTAL REFERENCES

S1. Zhang, X.; King-Smith, E.; Renata, H. Angew. Chemie—Int. Ed. 2018, 57, 5037-5041.
S2. Zwick, C. R.; Sosa, M. B.; Renata, H. J. Am. Chem. Soc. 2021, 143, 1673-1679.
S3. Stein, M. L.; Beck, P.; Kaiser, M.; Dudler, R.; Becker, C. F. W.; Groll, M. Proc. Natl. Acad. Sci. U.S.A 2012, 109, 18367-18371.
S4. Baud, D.; Saaidi, P. L.; Monfleur, A.; Harari, M.; Cuccaro, J.; Fossey, A.; Besnard, M.; Debard, A.; Mariage, A.; Pellouin, V.; et al. ChemCatChem 2014, 6, 3012-3017.
S5. Melaugh, G.; Giri, N.; Davidson, C. E.; James, S. L.; Del Pópolo, M. G. Phys. Chem. Chem. Phys. 2014, 16, 9422-9431.
S6. Amatuni, A.; Shuster, A.; Adibekian, A.; Renata, H. Cell Chem. Biol. 2020, 27, 1318-1326.
S7. Imker, H. J.; Krahn, D.; Clerc, J.; Kaiser, M.; Walsh, C. T. Chem. Biol. 2010, 17, 1077-1083.
S8. (a) Delaunay, D.; Toupet, L.; Le Corre, M. J. Org. Chem. 1995, 60, 6604-6607. (b) Franck, X.; Langlois, E.; Outurquin, F. Synthesis (Stuttg). 2007, 4, 719-724.
S9. Patel, J.; Clave, G.; Renard, P. Y.; Franck, X. Angew. Chemie 2008, 120, 4292-4295.
S10. Sakai, A.; Xiang, D. F.; Xu, C.; Song, L.; Yew, W. S.; Raushel, F. M.; Gerlt, J. A. Biochemistry 2006, 45, 4455-4462.
S11. Hibi, M.; Kasahara, T.; Kawashima, T.; Yajima, H.; Kozono, S.; Smirnov, S. V.; Kodera, T.; Sugiyama, M.; Shimizu, S.; Yokozeki, K.; et al. Adv. Synth. Catal. 2015, 357, 767-774.
S12. Prechter, A.; Heinrich, M. R. Synthesis (Stuttg). 2011, No. 10, 1515-1525.
S13. Pitre, S. P.; McTiernan, C. D.; Ismaili, H.; Scaiano, J. C. ACS Catal. 2014, 4, 2530-2535.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

The compounds provided herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds provided herein, even though only a single tautomeric form may be represented. All such isomeric forms of such compounds herein are expressly included in the present invention. All crystal forms and polymorphs of the compounds provided herein are expressly included in the present invention. All hydrate and solvate forms of the compounds provided herein are expressly included in the present invention. Also embodied are extracts and fractions comprising compounds provided herein. The term “isomers” is intended to include diastereoisomers, enantiomers, regioisomers, structural isomers, rotational isomers, tautomers, and the like. All such isomers of such compounds herein are expressly included in the present invention. For compounds which contain one or more stereogenic centers, e.g., chiral compounds, the methods provided herein may be carried out with an enantiomerically enriched compound, a racemate, or a mixture of diastereomers.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(Rb)₂)₂, —OP(═O)(N(R^{bb})₂)₂, —NR^{bb}P(═O)(R^{aa})₂—NR^{bb}P(═O)(OR^{cc})₂—NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂—OP(R^{cc})₃⁺X⁻, —OP(OR^{cc})₂, —OP(OR^{cc})₃⁺X⁻, —OP(R^{cc})₄, —OP(OR^{cc})₄, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein X⁻ is a counterion;
or two geminal hydrogens on a carbon atom are replaced with the group ═O, ═S, ═NN(R^{bb})₂, —NNR^{bb}C(═O)R^{aa}, —NNR^{bb}C(═O)OR^{aa}, —NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R′, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —N^{bb}CO₂R^{aa}, or —NR^{bb}C(═O)N(R^{bb})₂ In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —N^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “proteasome” as used herein includes, without limitation, those proteasomes that are expressed as “standard” or “constitutive” proteasomes, immunoproteasomes, or thymoproteasomes.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### Embodiments

One aspect of the invention is any compound, or salt thereof, described herein. Another aspect is a method of treating a disease, disorder, or symptom thereof, in a subject, comprising administration to the subject of a compound, or salt thereof, herein. Another aspect is a method of inhibiting a proteasome in a subject, comprising administration to the subject of a compound, or salt thereof, herein. Another aspect is a method of making a compound, or salt thereof, described herein using one or more reagents, chemical transformations, or chemical intermediate compounds as described herein.

### Further Embodiments Include:

1. A compound, or salt thereof, that is selected from compounds 40-51 or S11-S39, or salt thereof.
2. A compound, or salt thereof, that is selected from compounds 40-51 or S39, or salt thereof.
3. A pharmaceutical composition comprising a compound of any of compounds 40-51 or S11-S39, or salt thereof, and a pharmaceutically acceptable excipient.
4. A pharmaceutical composition comprising a compound of any of compounds 40-51 or S39, or salt thereof, and a pharmaceutically acceptable excipient.
5. A method of treating cancer in a subject, comprising administering any of compounds 40-51 or S11-S39, or salt thereof, to the subject.
6. A method of treating cancer in a subject, comprising administering any of compounds 40-51 or S39, or salt thereof, to the subject.
7. The method of any one of embodiments 5 or 6, wherein the cancer is multiple myeloma or solid tumor.
8. A method of inhibiting a proteasome in a subject, comprising administering any of compounds 40-51 or S11-S39, or salt thereof, to the subject.
9. A method of inhibiting a proteasome in a subject, comprising administering any of compounds 40-51 or S39, or salt thereof, to the subject.
10. The method of any one of embodiments 8 or 9, wherein the proteasome is the yeast core particle (CP) β5 subunit.
11. The method of any one of embodiments 8 or 9, wherein the proteasome is the yeast core particle (CP) β2 subunit.
12. The method of any one of embodiments 8 or 9, wherein the proteasome is the yeast core particle (CP) β5 subunit and the yeast core particle (CP) β2 subunit.
13. A method of inhibiting a proteasome, comprising contacting a compound of any of compounds 40-51 or S11-S39, or salt thereof, with a proteasome.
14. A method of inhibiting a proteasome, comprising contacting a compound of any of compounds 40-51 or S39, or salt thereof, with a proteasome.
15. A method of making compound 40-51 and S39, comprising treating compound S17 with TFA, and the resulting product S18 with DMTMMT, and the resulting product S19 with compound S20, to provide compound 40-51 and S39.
16. A compound of formula:

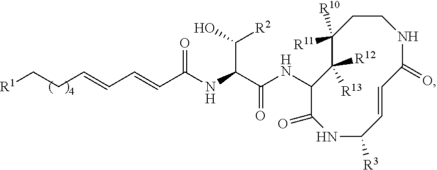

or salt thereof, wherein:
R¹ is optionally substituted alkyl, optionally substituted cycloalkyl, or optionally substituted aryl;
R² is optionally substituted alkyl or optionally substituted aryl;
R³ is optionally substituted alkyl; and
each of R¹⁰, R¹¹, R¹², and R¹³ is hydrogen or —OR′, or R¹⁰ and R¹¹ are taken together to form ═O, or R¹² and R¹³ are taken together to form ═O, wherein each instance of R′ is independently hydrogen, optionally substituted alkyl, or optionally substituted acyl;
provided that if R² is —CH₃, R³ is —CH₃, R¹⁰ is hydrogen, R¹¹ is hydrogen or —OH, R¹² is hydrogen, and R¹³ is hydrogen, R¹ is not —CH(CH₃)₂ or —CH₂CH₃.

1. A compound, or salt thereof, that is selected from compounds 40-51 or S11-S39, or salt thereof.

2. The compound, or salt thereof, of claim 1, wherein the compound, or salt thereof, is selected from compounds 40-51 or S39, or salt thereof.

3. A method of treating cancer in a subject, comprising administering any of compounds 40-51 or S11-S39, or salt thereof, to the subject.

4. A method of inhibiting a proteasome in a subject, comprising administering any of compounds 40-51 or S11-S39, or salt thereof, to the subject.

5. A method of inhibiting a proteasome, comprising contacting a compound of any of compounds 40-51 or S11-S39, or salt thereof, with a proteasome.

6. A method of making compound 40-51 and S39, comprising treating compound S17 with TFA, and the resulting product S18 with DMTMMT, and the resulting product S19 with compound S20, to provide compound 40-51 and S39.

7. A compound of formula: 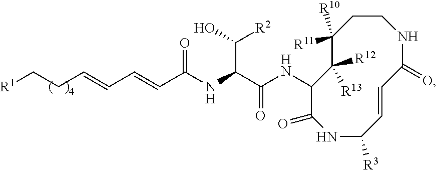
or salt thereof, wherein:
R¹ is optionally substituted alkyl, optionally substituted cycloalkyl, or optionally substituted aryl;
R² is optionally substituted alkyl or optionally substituted aryl;
R³ is optionally substituted alkyl; and
each of R¹⁰, R¹¹, R¹², and R¹³ is hydrogen or —OR′, or R¹⁰ and R¹¹ are taken together to form ═O, or R¹² and R¹³ are taken together to form ═O, wherein each instance of R′ is independently hydrogen, optionally substituted alkyl, or optionally substituted acyl;
provided that if R² is —CH₃, R³ is —CH₃, R¹⁰ is hydrogen, R¹¹ is hydrogen or —OH, R¹² is hydrogen, and R¹³ is hydrogen, R¹ is not —CH(CH₃)₂ or —CH₂CH₃.
